(19) United States
(12) Patent Application Publication
(10) Pub. No.: US 2011/0033393 A1
(43) Pub. Date: Feb. 10, 2011

(54) Hydrazone Compounds and Their Use

(76) Inventors: Philip STEIN, Pennington, NJ (US); Robert DAINES, Lansdale, PA (US); Dennis SPROUS, Ware, MA (US); Harold O'GRADY, Franklin Park, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/778,906

(22) Filed: May 12, 2010

Related U.S. Application Data
(60) Provisional application No. 61/177,620, filed on May 12, 2009.

(51) Int. Cl. A61K 8/42; C07C 251/86; A61K 31/165; C12N 5/00; A61P 3/10; A61P 3/04; A61P 5/50; A61P 1/00; C07D 209/20; A61K 31/405; A61K 8/49; A61Q 11/00; C07D 401/12; A61K 31/4709; A61K 31/47; C07D 471/04; A61K 31/437; A61K 31/4439; A61K 31/4184; C07D 235/16; A61K 31/501; C07D 403/12; A61K 31/497; A61K 31/502; C07D 413/12; A61K 31/538; A23L 1/226

(52) U.S. Cl. 424/49; 564/149; 514/615; 435/375; 548/496; 514/419; 546/176; 514/314; 514/311; 546/113; 514/300; 546/277.4; 514/339; 514/394; 548/309.7; 514/252.06; 544/238; 544/405; 514/255.05; 514/248; 544/235; 544/105; 514/230.5; 426/650

(57) ABSTRACT

The present invention relates to hydrazone compounds of Formula I:

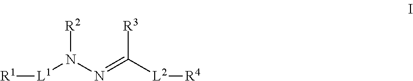

and pharmaceutically acceptable salts and stereoisomers thereof, wherein R¹, R², R³, R⁴, L¹, and L² are defined as set forth in the specification. The invention is also directed to the use of compounds of Formula I as inhibitors of TRPM5 protein.

### BACKGROUND OF THE INVENTION

1. Field of the Invention

The present invention relates to novel hydrazone compounds and the use of these compounds as inhibitors of a transient receptor potential cation channel, subfamily M, member 5 (TRPM5) protein.

2. Background Art

Taste perception plays a critical role in both the nutritional status of human beings and the basic survival of animals. Margolskee, R. F., J. Biol. Chem. 277:1-4 (2002); Avenet, P. and Lindemann, B., J. Membrane Biol. 112:1-8 (1989). The task of taste perception is carried out by taste receptor cells (TRCs). TRCs have the ability to perceive the multitude of compounds that are associated with a given taste and then convert that perception to a signal that is deciphered by the brain, resulting in the sensation of sweet, bitter, sour, salty, or umami (savory) taste.

TRCs are polarized epithelial cells, meaning that they have specialized apical and basolateral membranes. A taste bud contains approximately 60 to 100 TRCs. Each TRC has a portion of its membrane exposed on the mucosal surface of the tongue. Kinnamon, S. C., TINS 11:491-496 (1988). Sensory transduction is initiated by sapid molecules, or “tastants,” that interact with microvillar processes on the apical membrane of TRCs. The tastants bind specific membrane receptors, resulting in a voltage change across the cell membrane. In turn, this depolarizes, or changes the electric potential, of the cell, causing transmitter release and excitation of primary gustatory nerve fibers.

One recently discovered transmembrane protein, TRPM5, has been shown to be essential for taste transduction. Perez et al., Nature Neuroscience 5:1169-1176 (2002); Zhang et al., Cell 112:293-301 (2003). This protein is a member of the transient receptor potential (TRP) family of ion channels, forms a channel through the membrane of the taste receptor cell, and is believed to be activated by stimulation of a receptor pathway coupled to phospholipase C and by IP₃-mediated Ca²⁺ release. The opening of this channel is dependent on a rise in Ca²⁺ levels. Hofmann et al., Current Biol. 13:1153-1158 (2003). The activation of this channel leads to depolarization of the TRC, which in turn leads to transmitter release and excitation of primary gustatory nerve fibers.

Because TRPM5 is a necessary part of the taste-perception machinery, its inhibition prevents an animal from sensing particular tastes. Although taste perception is a vital function, the inhibition of undesirable tastes is beneficial under certain circumstances. For example, many active pharmaceutical ingredients of medicines produce undesirable tastes, such as a bitter taste. Inhibition of the bitter taste produced by the medicine may lead to improved acceptance by the patient.

Traditionally, sweeteners and flavorants have been used to mask the bitter taste of pharmaceuticals. The sweetener or flavorant is known to activate other taste pathways and at sufficiently high concentration this serves to mask the bitter taste of the pharmaceutical. However, this approach has proved ineffective at masking the taste of very bitter compounds. Microencapsulation in a cellulose derivative has also been used to mask the bitter taste of pharmaceuticals. However, this approach prevents rapid oral absorption of the pharmaceutical.

A number of other methods have been suggested to inhibit, alter, or mask unwanted tastes, including the use of 5′-adenosine monophosphate (AMP) and 5′-inosine monophosphate (IMP) as potential bitterness inhibitors. See U.S. Pat. No. 6,540,978. However, the presently available compounds are lacking in desirable characteristics.

Another aspect of taste is its role in food intake. Studies have shown increased food intake as palatability increased. Sorensen, et al., Int. J. Obes. Relat. Metab. Disord. 27(10):1152-66 (2003). For instance, certain drugs, such as antihypertensives and antihyperlipidemics, have been reported to produce untoward alterations in taste and may result in decreased food intake. Doty, et al., J Hypertens. 21(10):1805-13 (2003). Taste impairment has also been associated with radiation treatments for head and neck cancer and this taste impairment has been considered to be one of the factors associated with reduced appetite and altered patterns of food intake. V is sink, et al., Crit. Rev. Oral Biol. Med. 14(3):213-25 (2003). Decreased food consumption has also been correlated with loss of taste sensations in the elderly. Shiffman, S. S., J. Am. Med. Ass'n 278(16):1357-1362 (1997).

Diabetes mellitus is a syndrome characterized by abnormal insulin production, increased urinary output and elevated blood glucose levels. There are two major subclasses which can be described based on the level of insulin production by a person's pancreatic beta cells. One is insulin-dependent diabetes mellitus (IDDM, or Type 1), formerly referred to as juvenile onset diabetes since it was evident early in life. In Type 1 Diabetes, little or no insulin is produced as the pancreatic beta cells have been destroyed by the body's own immune system. Between 5-10% of all diabetics have IDDM (American Diabetes Association. Diabetes 1996 Vital Statistics. Rockville, Md.: American Diabetes Association, 1996.) The other type is non-insulin dependent diabetes mellitus (NIDDM, or Type 2), often referred to as maturity-onset diabetes. In Type 2 Diabetes, pancreatic beta cells produce insulin but not in sufficient quantities to maintain healthy blood glucose levels. Type 2 Diabetes results from the deterioration in the molecular machinery that mediates the effectiveness of insulin function on cells (e.g., insulin resistance and inadequate insulin release). Between 90-95% of all diabetics are NIDDM (Harris, M. I., Cowie, C. C., Stern, M. P. eds. Diabetes in America, 2nd. ed. National Institutes of Health. National Institute of Diabetes and Digestive and Kidney Diseases. NIH Publication No. 95-1468, 1995).

Type 2 diabetes is a significant healthcare problem, and its incidence is on the rise. Between 1990 and 1998, the prevalence of NIDDM in the United States increased by 33 percent, to about 13 million persons. An additional 5 million persons are presumed to have undiagnosed NIDDM, while another 14 million persons have impaired glucose tolerance. Direct medical costs associated with diabetes were $44 billion in 1997, due mainly to hyperglycemia-related diabetic complications, including diabetic angiopathy, atherosclerosis, diabetic nephropathy, diabetic neuropathy, and diabetic ocular complications such as retinopathy, cataract formation, and glaucoma.

Resistance to the metabolic actions of insulin is one of the key features of non-insulin dependent diabetes. Insulin resistance is characterized by impaired uptake and utilization of glucose in insulin-sensitive target organs, for example, adipocytes and skeletal muscle, and by impaired inhibition of hepatic glucose output. The functional insulin deficiency and the failure of insulin to suppress hepatic glucose output result in fasting hyperglycemia. Pancreatic beta-cells compensate for the insulin resistance by secreting increased levels of insulin. However, the beta-cells are unable to maintain this high output of insulin, and, eventually, the glucose-induced insulin release falls, leading to the deterioration of glucose homeostasis and to the subsequent development of overt diabetes.

Other metabolic disorders associated with impaired glucose utilization and insulin resistance include insulin resistance syndrome (hereinafter “IRS”), which refers to the cluster of manifestations that include insulin resistance; hyperinsulinemia; non insulin dependent diabetes mellitus (NIDDM); arterial hypertension; central (visceral) obesity; and dyslipidemia.

The primary goal of insulin resistance therapy and thus diabetes therapy is to lower blood glucose levels so as to prevent acute and long-term disease complications. For some persons, modified diet and increased exercise may be successful therapeutic options for achieving the goal of glucose control. When modified diet and increased exercise are not successful, drug therapy using oral antidiabetic agents is initiated.

Control of insulin release is very important, as there are many living diabetes patients whose pancreas is not operating correctly. In some types of diabetes, the total level of insulin is reduced below that required to maintain normal blood glucose levels. In others, the required insulin is generated but only at an unacceptable delay after the increase in blood glucose levels. In others, the body is, for some reason, resistant to the effects of insulin. If the diabetes is poorly controlled, it can lead to diabetic complications. Diabetic complications are common in Type 2 patients with approximately 50% suffering from one or more complications at the time of diagnosis (Clark, C. M., Vinicor, F. Introduction: Risks and benefits of intensive management in non-insulin-dependent diabetes mellitus. The Fifth Regensrief Conference. Ann Intern Med, 124(1, pt 2), 81-85, 1996).

Exogenous insulin by injection is used clinically to control diabetes but suffers from several drawbacks. Insulin is a protein and thus cannot be taken orally due to digestion and degradation but must be injected. It is not always possible to attain good control of blood sugar levels by insulin administration. Insulin resistance sometimes occurs, requiring much higher doses of insulin than normal. Another shortcoming of insulin is that, while it may control hormonal abnormalities, it does not always prevent the occurrence of complications such as neuropathy, retinopathy, glomerulosclerosis, and cardiovascular disorders. Insulin regulates glucose homeostasis mainly by acting on two targets tissues: liver and muscle. Liver is the only site of glucose production, and skeletal muscle is the main site of insulin mediated glucose uptake.

There are several classes of drugs that are useful for treatment of Type 2 Diabetes: 1) insulin releasers, which directly stimulate insulin release, carrying the risk of hypoglycemia; 2) prandial insulin releasers, which potentiate glucose-induced insulin release and must be taken before each meal; 3) biguanides, including metformin, which attenuate hepatic gluconeogenesis (which is paradoxically elevated in diabetes); 4) insulin sensitizers, for example the thiazolidinedione derivatives rosiglitazone and pioglitazone, which improve peripheral responsiveness to insulin, but which have side effects like weight gain, edema, and occasional liver toxicity; and 5) insulin injections, which are often necessary in the later stages of Type 2 Diabetes when the islets have failed under chronic hyperstimulation. The effectiveness of current oral antidiabetic therapies is limited, in part, because of poor or limited glycemic control, or poor patient compliance due to unacceptable side effects. These side effects include edema weight gain, hypoglycemia, and even more serious complications.

Insulin secretagogues are standard therapy for Type 2 diabetics who have mild to moderate fasting hyperglycemia. Insulin secretors include sulfonylureas (SFUs) and the non-sulfonylureas, nateglinide and pepaglinide. The sulfonylureas are subdivided into two subcategories: the first generation agents, e.g., tolbutamide, chlorpropamide, tolazamide, acetohexamide, and the second generation agents, e.g., glyburide (glibenclamide), glipizide and gliclazide.

The insulin secretagogues have limitations that include a potential for inducing hypoglycemia, weight gain, and high primary and secondary failure rates. Approximately 10 to 20% of initially treated patients fail to show a significant treatment effect (primary failure). Secondary failure is demonstrated by an additional 20-30% loss of treatment effect after six months of treatment with insulin secretagogues. Insulin treatment is required in 50% of the insulin secretagogue responders after 5-7 years of therapy (Scheen et al., Diabetes Res. Clin. Pract. 6:533 543, 1989). Nateglinide and pepaglinide are short-acting drugs that need to be taken three times a day. They are used only for the control of post-prandial glucose and not for control of fasting glucose.

Treatment with sulfonylureas increases the risk of hypoglycemia (or insulin shock), which occurs if blood glucose levels fall below normal (UKPDS Group. UK Prospective Diabetes Study 33: Intensive blood-glucose control with sulphonylureas or insulin compared with conventional treatment and risk of complications in patients with type 2 diabetes. Lancet, 352, 837-853 (1998)).

As a result, there is a need for additional antidiabetic agents which can be used to stimulate insulin secretion.

Treatment with a gastrointestinal protein hormone is potentially another way to treat diabetes mellitus. Gastrointestinal protein hormones, including, but not limited to, glucose-dependent insulinotropic polypeptide (GIP) and glucagon-like peptide-1 (GLP-1), stimulate insulin synthesis and secretion from the beta cells of the islets of Langerhans after food intake, thereby lowering blood glucose levels. Further, oral administration of glucose has long been known to increase insulin secretion more than intraveousintravenous glucose administration does, despite similar plasma glucose concentration. Scow et al., Am J. Physiol., 179(3):435-438 (1954). Such an effect, called the incretin effect, provides the basis for regulating glucose disposal and treatment of diabetes and its related disease.

The most potent gastrointestinal protein hormone is GLP-1, which is initially a 37-amino acid peptide and a product of proglucagon. A subsequent endogenous cleavage between the sixth and seventh position produces the biologically active GLP-1 (7-37) peptide. GLP-1 is secreted from the L-type enteroendorcrine cells in the luminal surface of the gut upon glucose intake. GLP-1 acts through a G-protein-coupled cell-surface receptor (GLP-1R) and may be regulated by T1R taste receptors and gustducin. See Kokrashvili et al. AChemS XXIX Abstract, 246 (2007). Studies have shown that α-gustducin couples sweet receptor T1R3 in sugar- and sweetener-stimulated secretion of GLP-1 from the L-type enteroendocrine cells. See Jang et al. Proc. Natl. Acad. Sci. USA, 104(38): 15069-15074; Margolskee, et al., Proc. Natl. Acad. Sci. USA 104(38):15075-15080 (2007). GLP-1 possesses several physiological functions; for example, 1) it stimulates insulin synthesis from the pancreatic islet cells in a glucose-dependent manner, thereby lowering blood glucose levels; 2) it decreases glucagon secretion from the pancreas; 3) it increases beta cell mass and insulin gene expression; 4) it inhibits gastric secretion and emptying; 5) it dose-dependently inhibits food intake by increasing satiety; and 6) it promotes weight loss. Several roles for GLP-1 are described by U.S. Pat. No. 6,583,118, U.S. Pat. No. 7,211,557, U.S. Patent Appl. Pub. No. 2005/0244810, Deacon, Regulatory Peptides 128: 117-124 (2005); and Turton et al., Nature, 379, 69-72 (1996).

At present, while there are a number of agents that are or have been on the market to reduce appetite and food intake, such as amphetamine derivatives and fenfluramine, many have serious side effects. More selective approaches, e.g., neuro-regulation via peptide mimetics/antagonists, are still in developmental phases.

U.S. Patent Application Publication No. US 2007/0207093 A1 to Bryant et al. describes the use of certain hydrazone derivatives for inhibiting certain taste functions and perceptions and related uses.

U.S. Patent Application Publication No. US 2008/0306030 A1 to Lee et al. describes methods of enhancing insulin release, enhancing GLP-1 release, increasing insulin sensitivity, increasing beta cells mass and insulin gene expression, decreasing gastric secretion, decreasing gastric emptying, and decreasing glucagon secretion by administering to a subject an effective amount of a TRPM5 inhibitor, such as a hydrazone derivative. The publication also describes methods of treating diabetes mellitus, insulin resistance syndrome, and hyperglycemia, and obesity by administering to a subject an effective amount of a TRPM5 inhibitor, such as a hydrazone derivative.

### SUMMARY OF THE INVENTION

The present invention is related to the use of hydrazone compounds represented by Formula I, below, and the pharmaceutically acceptable salts, prodrugs, and stereoisomers thereof, as inhibitors of TRPM5 protein.

Compounds useful in the present invention have not been heretofore reported. Thus, one aspect of the present invention is directed to novel compounds of Formula I, as well as their pharmaceutically acceptable salts, prodrugs, and stereoisomers.

Another aspect of the present invention is directed to the use of the novel compounds of Formula I, and their pharmacautically acceptable salts and isomers, as inhibitors of TRPM5 protein.

A further aspect of the present invention is to provide a pharmaceutical composition, comprising a compound of Formula I, or a pharmaceutically acceptable salt or stereoisomer thereof, in a mixture with one or more pharmaceutically acceptable carriers.

A further aspect of the present invention is to provide a food product, comprising a compound of Formula I, or a pharmaceutically acceptable salt or stereoisomer thereof, and one or more food ingredients.

A further aspect of the present invention is to provide a cosmetic product, comprising a compound of Formula I, or a pharmaceutically acceptable salt or stereoisomer thereof, and one or more cosmetic ingredients.

A further aspect of the present invention is to provide a dental hygienic product, comprising a compound of Formula I, or a pharmaceutically acceptable salt or stereoisomer thereof, and one or more dental hygienic ingredients.

The present invention is also directed to a method of inhibiting a taste, said method comprising administering to a subject in need of said taste inhibiting one or more compounds of Formula I, or a pharmaceutically acceptable salt or stereoisomer thereof.

An aspect of the present invention is directed to a method of inhibiting the depolarization of a taste receptor cell, said method comprising contacting said taste receptor cell with a compound of Formula I, or a pharmaceutically acceptable salt or stereoisomer thereof.

An additional aspect of the present invention is directed to a method of decreasing the palatability of food and its intake, comprising administering one or more compounds of Formula I, or a pharmaceutically acceptable salt or stereoisomer thereof, to a subject in need of such treatment.

A further aspect of the invention is to provide a method of preparing an improved pharmaceutical composition, wherein the improvement comprises adding to a pharmaceutical composition one or more compounds of Formula I, or a pharmaceutically acceptable salt or stereoisomer thereof.

A further aspect of the invention is to provide a method of preparing an improved cosmetic product, wherein the improvement comprises adding to a pharmaceutical composition one or more compounds of Formula I, or a pharmaceutically acceptable salt or stereoisomer thereof.

A further aspect of the invention is to provide a method of enhancing insulin release from a cell, comprising contacting said cell with an effective amount a compound of Formula I, or a pharmaceutically acceptable salt or stereoisomer thereof.

A further aspect of the invention is to provide a method of enhancing insulin release in a mammal, comprising administering to the mammal in need of said enhanced insulin release an effective amount of a compound of Formula I, or a pharmaceutically acceptable salt or stereoisomer thereof.

A further aspect of the invention is to provide a method of treating diabetes mellitus in a mammal, comprising administering to the mammal in need of said treatment an effective amount of a compound of Formula I, or a pharmaceutically acceptable salt or stereoisomer thereof.

A further aspect of the invention is to provide a method of treating insulin resistance syndrome in a mammal, comprising administering to the mammal in need of said treatment an effective amount of a compound of Formula I, or a pharmaceutically acceptable salt or stereoisomer thereof.

A further aspect of the invention is to provide a method of treating hyperglycemia in a mammal, comprising administering to the mammal in need of said treatment an effective amount of a compound of Formula I, or a pharmaceutically acceptable salt or stereoisomer thereof.

A further aspect of the invention is to provide a method of enhancing GLP-1 release in a mammal, comprising administering to the mammal in need of said enhanced GLP-1 release an effective amount of a compound of Formula I, or a pharmaceutically acceptable salt or stereoisomer thereof.

A further aspect of the invention is to provide a method of enhancing GLP-1 release from a cell, comprising contacting said cell with an effective amount of a compound of Formula I, or a pharmaceutically acceptable salt or stereoisomer thereof.

A further aspect of the invention is to provide a method of decreasing gastric secretion and emptying in a mammal, comprising administering to the mammal in need thereof an effective amount of a compound of Formula I, or a pharmaceutically acceptable salt or stereoisomer thereof.

A further aspect of the invention is to provide a method of inhibiting food intake in a mammal, comprising administering to a mammal in need thereof an effective amount of a compound of Formula I, or a pharmaceutically acceptable salt or stereoisomer thereof.

A further aspect of the invention is to provide a method of decreasing glucagon secretion in a mammal, comprising administering to the mammal in need thereof an effective amount of a compound of Formula I, or a pharmaceutically acceptable salt or stereoisomer thereof.

A further aspect of the invention is to provide a method of enhancing insulin sensitivity in a mammal, comprising administering to a subject in need thereof an effective amount of a compound of Formula I, or a pharmaceutically acceptable salt or stereoisomer thereof.

A further aspect of the invention is to provide a method of treating obesity in a mammal, comprising administering to the mammal in need thereof an effective amount of a compound of Formula I, or a pharmaceutically acceptable salt or stereoisomer thereof.

A further aspect of the invention is to provide a method of increasing beta cell mass of the islets of Langerhans in a mammal, comprising administering to the mammal in need thereof an effective amount of a compound of Formula I, or a pharmaceutically acceptable salt or stereoisomer thereof.

A further aspect of the invention is to provide a method of increasing insulin gene expression in a mammal, comprising administering to the mammal in need thereof an effective amount of a compound of Formula I, or a pharmaceutically acceptable salt or stereoisomer thereof.

Additional embodiments and advantages of the invention will be set forth in part of the description that follows, and will flow from the description, or may be learned by practice of the invention. The embodiments and advantages of the invention will be realized and attained by means of the elements and combinations particularly pointed out in the appended claims.

It is to be understood that both the foregoing summary and the following detailed description are exemplary and explanatory only and are not restrictive of the invention, as claimed.

### BRIEF DESCRIPTION OF THE DRAWINGS/FIGURES

FIG. 1 depicts graphically the results of Example 71 illustrating the effect of the compound of Example 10 on insulin secretion in the insulinoma cell like Beta-TC-6.

FIG. 2 depicts graphically the results of Example 72 illustrating the effect of the compound of Example 10 on insulin secretion in isolated rat primary islets.

FIG. 3 depicts graphically the results of Example 73 illustrating the effect of the compound of Example 10 on GLP-1 secretion in the enteroendocrine cell line GLUTag.

### DETAILED DESCRIPTION OF THE INVENTION

One aspect of the present invention is based on the use of compounds of Formula I, and the pharmaceutically acceptable salts and stereoisomers thereof, as inhibitors of TRPM5 protein. In view of this property, compounds of Formula I, and pharmaceutically acceptable salts and stereoisomers thereof, are useful for inhibiting the activity of a taste modulating protein, enhancing insulin secretion, treating diabetes mellitus, treating, preventing or controlling hyperglycemia and/or insulin resistance in a mammal, enhancing GLP-1 release from a cell, decreasing gastric secretion and emptying, inhibiting food intake, decreasing glucagon secretion, enhancing insulin sensitivity, increasing beta cells mass and insulin gene expression, and treating obesity in a mammal.

In one embodiment, the compounds useful in this aspect of the invention are compounds represented by Formula I:

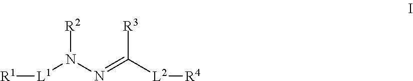

and pharmaceutically acceptable salts and stereoisomers thereof, wherein:

R² and R³ are each independently hydrogen or alkyl;

R⁴ is aryl, a 6-membered heteroaryl having at least one nitrogen atom, or a 9-10-membered heteroaryl having at least one nitrogen atom, each of which is optionally substituted; or

R⁴ is phenyl, wherein two adjacent carbon atoms of the phenyl ring form a bridge —O—(CH₂)ᵣ—O— or —(NR²⁶)—(CH₂)ₛ—O— to form a fused bicyclic ring; wherein r and s are 1, 2, or 3, and R²⁶ is H or C₁₋₄ alkyl, and wherein the bicyclic ring is optionally substituted;

L² is absent; and

R¹-L¹- is selected from the following:

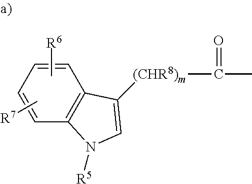

wherein:

R⁵ is C₁₋₅ alkyl;

R⁶ and R⁷, which can be present in either ring of the indol-3-yl group, are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl;

R⁸ is independently hydrogen or C₁₋₆ alkyl; and

m is 1, 2, or 3;

b)
R^{1d}-cyclopropyl-C(═O)—, wherein

R^{1d} is optionally substituted phenyl or optionally substituted 6-10 membered heteroaryl having at least one nitrogen atom,

c) R^{1a}—Z—(CHR¹²)ₙ—C(O)—; wherein
R^{1a} is optionally substituted aryl,
R¹² is independently hydrogen or C₁₋₆ alkyl,
Z is S, SO, or SO₂, and
n is 1, 2, or 3;

d) R^{1b}—(CHR¹³)ₚ—NH—C(O)—; wherein
R^{1b} is alkyl, cycloalkyl, aryl or heteroaryl, each of which is optionally substituted,
R¹³ is independently hydrogen or C₁₋₆ alkyl, and
p is 0, 1, 2, or 3; and

e) R^{1c}—(CHR¹⁴)_{q}—C(O)—, wherein
R^{1c} is optionally substituted indol-1-yl, optionally substituted pyrrolo[2,3-b]pyridin-1-yl, optionally substituted benzoimidazol-1-yl, optionally substituted quinolinyl, or optionally substituted pyridinyl;
R¹⁴ is independently hydrogen or C₁₋₆ alkyl; and
q is 1, 2, or 3.

One group of compounds useful in this aspect of the invention are compounds of Formula I as defined above, wherein R¹-L¹- is R^{1d}-cyclopropyl-C(═O)—, with the following provisos:

1) when R² and R³ are hydrogen, R⁴ is unsubstituted phenyl or phenyl substituted with other than one of arylalkoxy or heteroarylalkoxy, and R^{1d} is optionally substituted phenyl, then R^{1d} is substituted with at least one haloalkyl, or

2) when R^{1d} is optionally substituted phenyl, then R⁴ is other than optionally substituted isoquinolinyl.

In one embodiment, the compounds useful in this aspect of the invention are compounds represented by Formula I:

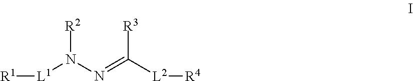

and pharmaceutically acceptable salts and stereoisomers thereof, wherein:

R² and R³ are each independently hydrogen or alkyl;

R⁴ is aryl or a 9-10-membered heteroaryl having at least one nitrogen atom, each of which is optionally substituted;

L² is absent; and

R¹-L¹- is selected from the following:

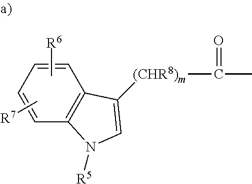

wherein:

R⁵ is C₁₋₅ alkyl;

R⁶ and R⁷, which can be present in either ring of the indol-3-yl group, are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino;

R⁸ is independently hydrogen or C₁₋₆ alkyl; and

m is 1, 2, or 3;

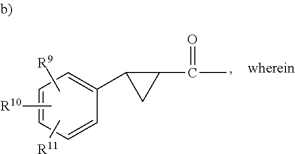

R⁹, R¹⁰ and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino,

provided that when R² is hydrogen, then R⁹ is haloalkyl;

c) R^{1a}—S—(CHR¹²)ₙ—C(O)—; wherein
R^{1a} is optionally substituted aryl,
R¹² is independently hydrogen or C₁₋₆ alkyl, and
n is 1, 2, or 3;

d) R^{1b}—(CHR¹³)ₚ—NH—C(O)—; wherein
R^{1b} is alkyl, cycloalkyl, aryl or heteroaryl, each of which is optionally substituted,
R¹³ is independently hydrogen or C₁₋₆ alkyl, and
p is 0, 1, 2, or 3; and

e) R^{1c}—(CHR¹⁴)_{q}—C(O)—, wherein
R^{1c} is optionally substituted indol-1-yl;
R¹⁴ is independently hydrogen or C₁₋₆ alkyl; and
q is 1, 2, or 3.

In one embodiment, compounds useful in the present invention are compounds of Formula I, wherein R¹-L¹- is

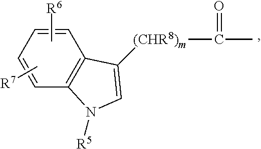

wherein

R⁵ is C₁₋₅ alkyl;

R⁶ and R⁷, which can be present in either ring of the indol-3-yl group, are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl;

R⁸ is independently hydrogen or C₁₋₆ alkyl; and

m is 1, 2, or 3.

Useful compounds of the present invention include those, where m is 1 or 2 and R⁸ is independently hydrogen, methyl or ethyl. Preferably, —(CHR⁸)ₘ— is —CH₂—, —CH₂CH₂—, —CH(CH₃)—, —CH(CH₃)CH₂—, or —CH₂CH(CH₃)—.

Useful compounds of the invention include those where R⁵ is C₁₋₄ alkyl, and especially methyl or ethyl.

Useful compounds of the present invention include those where R⁶ and R⁷ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino, and preferably selected from the group consisting of hydrogen, halogen, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Useful compounds of the present invention include those where R⁶ and R⁷ are both hydrogen.

In one embodiment, compounds useful in the present invention are compounds of Formula I, wherein R¹-L¹- is
R^{1d}-cyclopropyl-C(═O)—,

wherein R^{1d} is optionally substituted phenyl or optionally substituted 6-10 membered heteroaryl having at least one nitrogen atom,

provided that 1) when R² and R³ are hydrogen, R⁴ is unsubstituted phenyl or phenyl substituted with other than one of arylalkoxy or heteroarylalkoxy, and R^{1d} is optionally substituted phenyl, then R^{1d} is substituted with at least one haloalkyl, or 2) when R^{1d} is optionally substituted phenyl, then R⁴ is other than optionally substituted isoquinolinyl.

In one embodiment, R^{1d} is optionally substituted 6-membered or 9-10-membered heteroaryl having at least one nitrogen atom. Useful 6- or 9-10-membered heteroaryl groups include quinolinyl (e.g., quinolin-2-yl, quinolin-3-yl, or quinolin-6-yl), pyridinyl (e.g., pyridin-2-yl, pyridin-3-yl, or pyridin-4-yl), isoquinolinyl (e.g., isoquinolin-1-yl, isoquinolin-3-yl, or isoquinolin-6-yl), benzimidazolyl (e.g., benzimidazol-2-yl, benzimidazol-5-yl, or benzimidazol-6-yl), pyrrolopyridinyl (e.g., pyrrolo[2,3-b]pyridin-1-yl), indazolyl (e.g., indazol-1-yl, indazol-3-yl, indazol-5-yl, or indazol-6-yl), benzothiazolyl (e.g., benzothiazol-2-yl, benzothiazol-5-yl, or benzothiazol-6-yl), cinnolinyl (e.g., cinnolin-3-yl, cinnolin-4-yl, cinnolin-6-yl, cinnolin-7-yl, or cinnolin-8-yl), phthalazinyl (e.g., phthalazin-1-yl, phthalazin-6-yl, phthalazin-7-yl, or phthalazin-8-yl), quinazolinyl (e.g., quinazolin-2-yl, quinazolin-6-yl, or quinazolin-7-yl), quinoxalinyl (e.g., quinoxalin-2-yl, quinoxalin-6-yl, or quinoxalin-7-yl), and naphthyridinyl (e.g., 1,5-naphthyridinyl or 1,8-naphthyridinyl, such as 1,8-naphthyridin-2-yl, 1,8-naphthyridin-3-yl, 1,8-naphthyridin-4-yl, and 1,5-naphthyridin-2-yl). In one embodiment, the 6- or 9-10-membered heteroaryl group is unsubstituted. In one embodiment, the 6- or 9-10-membered heteroaryl group is substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl; preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, di(C₁₋₄)alkylamino, C₁₋₆ alkylamino(C₁₋₄)alkyl, and di(C₁₋₄)alkylamino(C₁₋₄)alkyl. Suitable optional substituents include halogen, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino.

In one embodiment, R^{1d} is optionally substituted 6- or 9-10-membered heteroaryl having at least one nitrogen atom and R⁴ is optionally substituted aryl, such as optionally substituted phenyl.

In one embodiment, R^{1d} is optionally substituted phenyl. In this embodiment, compounds useful in the present invention are compounds of Formula I, wherein R¹-L¹- is

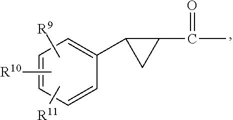

wherein

R⁹, R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl. In one embodiment, when R² is hydrogen, then R⁹ is haloalkyl. In one embodiment, compounds useful in the present invention are compounds of Formula I, wherein R² is hydrogen, R⁹ is haloalkyl, and R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino. In one embodiment, compounds useful in the present invention are compounds of Formula I, wherein R⁴ is unsubstituted phenyl or phenyl substituted with other than one of arylalkoxy or heteroarylalkoxy, R² and R³ are hydrogen, R⁹ is haloalkyl, and R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino. In this embodiment, R⁹ is preferably halo(C₁₋₆)alkyl, more preferably trifluoromethyl, trifluoroethyl, or trichloromethyl, and advantageously trifluoromethyl. Useful compounds in this aspect of the present invention include those where R¹⁰ and R¹¹ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. Preferably, R¹⁰ is hydrogen and R¹¹ is hydrogen, halogen, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, or dimethylamino. Useful compounds include those where R¹⁰ and R¹¹ both are hydrogen. Advantageously, R⁹ is attached to the 3-position of the phenyl ring. Preferably, R¹ is 3-trifluoromethylphenyl.

In another embodiment, compounds useful in the present invention are compounds of Formula I, wherein R² is alkyl, and R⁹, R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl. Useful compounds in this aspect of the present invention include those where R⁹, R¹⁰, and R¹¹ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(Cₕ₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino, and preferably each independently selected from the group consisting of hydrogen, halogen, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Useful compounds include those where R⁹ is hydrogen, and R¹⁰ and R¹¹ are each independently hydrogen, halogen, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, or dimethylamino. In one embodiment, useful compounds of the present invention include those where R⁹, R¹⁰ and R¹¹ are each hydrogen. In one embodiment, R⁹ is halo(C₁₋₆)alkyl, preferably trifluoromethyl, trifluoroethyl, or trichloromethyl, and advantageously trifluoromethyl, and R¹⁰ and R¹¹ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino, and preferably R¹⁰ and R¹¹ are both hydrogen.

In one embodiment, R^{1d} is optionally substituted phenyl, R⁴ is substituted phenyl, wherein at least one substituent is arylalkyloxy or heteroarylalkyloxy, wherein the aryl and heteroaryl groups can be optionally substituted. Suitable arylalkyloxy groups include optionally substituted phenyl(C₁₋₄)alkyloxy groups, such as phenylmethoxy, phenylethoxy, phenylpropoxy, and phenylbutoxy, and advantageously phenylmethoxy. Suitable heteroarylalkyloxy groups include optionally substituted heteroaryl(C₁₋₄)alkyloxy groups, such as heteroarylmethoxy, heteroarylethoxy, heteroarylpropoxy, and heteroarylbutoxy. Suitable heteroaryl groups include 6-membered heteroaryl groups having at least on nitrogen atom, such as pyridinyl, pyridazinyl, and pyrazinyl. Useful heteroarylalkyloxy groups include pyridin-2-ylmethoxy, pyridin-3-ylmethoxy, pyridin-4-ylmethoxy, pyridazin-3-ylmethoxy, and pyrazin-2-ylmethoxy.

In one embodiment, compounds useful in the present invention are compounds of Formula I, wherein R¹-L¹- is R^{1a}—Z—(CHR¹²)ₙ—C(O)—; wherein

R^{1a} is optionally substituted aryl,

R¹² is independently hydrogen or C₁₋₆ alkyl,

Z is S, SO, or SO₂; and

n is 1, 2, or 3.

Useful compounds of the present invention include those where n is 1 or 2, and R¹² is independently hydrogen, methyl or ethyl. Preferably, —(CHR¹²)ₙ— is —CH₂—, —CH₂CH₂—, —CH(CH₃)—, —CH(CH₃)CH₂—, or —CH₂CH(CH₃)—. In one embodiment, Z is S. In another embodiment, Z is SO. In another embodiment, Z is SO₂.

Useful compounds of the present invention include those where R^{1a} is phenyl, naphthyl or biphenyl, each of which is optionally substituted. Preferably, R^{1a} is phenyl, naphthyl, or biphenyl optionally substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl. In one embodiment, R^{1a} is unsubstituted phenyl or phenyl substituted with 1 or 2 substituents each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, di(C₁₋₄)alkylamino, C₁₋₄ alkylamino(C₁₋₆)alkyl, and di(C₁₋₄ alkyl)amino(C₁₋₆)alkyl. Preferably, R^{1a} is phenyl substituted with 1 or 2 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. In one embodiment, R⁴ is optionally substituted phenyl. In one embodiment, R⁴ is optionally substituted 6-10 membered heteroaryl having at least one nitrogen atom, such as pyridinyl or quinolinyl.

In one embodiment, compounds useful in the present invention are compounds of Formula I, wherein R¹-L¹- is R^{1b}—(CHR¹³)ₚ—NH—C(O)—; wherein

R^{1b} is alkyl, cycloalkyl, aryl or heteroaryl, each of which is optionally substituted,

R¹³ is independently hydrogen or C₁₋₆ alkyl, and

p is 0, 1, 2, or 3.

Useful compounds of the present invention include those where p is 1 or 2, and R¹³ is independently hydrogen, methyl or ethyl. Preferably, —(CHR¹³)ₚ— is —CH₂—, —CH₂CH₂—, —CH(CH₃)—, —CH(CH₃)CH₂—, or —CH₂CH(CH₃)—.

In one embodiment, useful compounds of the present invention include those where R^{1b} is optionally substituted alkyl. Preferably, R^{1b} is unsubstituted alkyl, more preferably unsubstituted C₁₋₆ alkyl.

In one embodiment, useful compounds of the present invention include those where R^{1b} is optionally substituted cycloalkyl. Preferably, R^{1b} is unsubstituted cycloalkyl, more preferably unsubstituted C₅₋₆ cycloalkyl.

In one embodiment, compounds useful in the present invention include those where R^{1b} is optionally substituted aryl. Preferably, the aryl group is phenyl, naphthyl or biphenyl. Useful compounds include those where R^{1b} is phenyl, naphthyl or biphenyl, each of which optionally substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl. In one embodiment, R^{1b} is unsubstituted phenyl or phenyl substituted with 1, 2, or 3 substituents each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. Useful compounds include those where R^{1b} is unsubstituted phenyl or phenyl substituted with 1, 2 or 3 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino.

In one embodiment, useful compounds of the present invention include those where R^{1b} is optionally substituted heteroaryl. In one embodiment, R^{1b} is unsubstituted heteroaryl. In another embodiment, R^{1b} is heteroaryl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylaminoalkyl). Useful compounds include those where R^{1b} is a 6-10-membered heteroaryl containing carbon atoms and 1-2 nitrogen atoms, wherein the heteroaryl is substituted with 1, 2, or 3 substituents each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino, and preferably the heteroaryl is substituted with 1, 2 or 3 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Suitable heteroaryl groups include indolyl (e.g., indol-3-yl and indol-1-yl), quinolinyl (e.g., quinolin-2-yl), pyridyl (e.g., pyridin-2-yl, pyridin-3-yl, and pyridin-4-yl), and pyrazinyl (e.g., pyrazin-2-yl and pyrazin-3-yl). Other suitable heteroaryl groups include isoquinolinyl (e.g., isoquinolin-1-yl, isoquinolin-3-yl, or isoquinolin-6-yl), benzimidazolyl (e.g., benzimidazol-2-yl, benzimidazol-5-yl, or benzimidazol-6-yl), pyrrolopyridinyl (e.g., pyrrolo[2,3-b]pyridin-1-yl), indazolyl (e.g., indazol-1-yl, indazol-3-yl, indazol-5-yl, or indazol-6-yl), benzothiazolyl (e.g., benzothiazol-2-yl, benzothiazol-5-yl, or benzothiazol-6-yl), cinnolinyl (e.g., cinnolin-3-yl, cinnolin-4-yl, cinnolin-6-yl, cinnolin-7-yl, or cinnolin-8-yl), phthalazinyl (e.g., phthalazin-1-yl, phthalazin-6-yl, phthalazin-7-yl, or phthalazin-8-yl), quinazolinyl (e.g., quinazolin-2-yl, quinazolin-6-yl, or quinazolin-7-yl), quinoxalinyl (e.g., quinoxalin-2-yl, quinoxalin-6-yl, or quinoxalin-7-yl), and naphthyridinyl (e.g., 1,5-naphthyridinyl or 1,8-naphthyridinyl, such as 1,8-naphthyridin-2-yl, 1,8-naphthyridin-3-yl, 1,8-naphthyridin-4-yl, and 1,5-naphthyridin-2-yl).

In one embodiment, compounds useful in the present invention are compounds of Formula I, wherein R¹-L¹- is R^{1c}—(CHR¹⁴)_{q}—C(O)—, wherein

R^{1c} is optionally substituted indol-1-yl, optionally substituted pyrrolo[2,3-b]pyridin-1-yl, optionally substituted benzoimidazol-1-yl, optionally substituted quinolinyl, or optionally substituted pyridinyl;

R¹⁴ is independently hydrogen or C₁₋₆ alkyl; and

q is 1, 2, or 3.

Useful compounds of the present invention include those where q is 1 or 2, and R¹⁴ is independently hydrogen, methyl or ethyl. Preferably, —(CHR¹⁴)_{q}— is —CH₂—, —CH₂CH₂—, —CH(CH₃)—, —CH(CH₃)CH₂—, or —CH₂CH(CH₃)—.

In one embodiment, R^{1c} is unsubstituted indol-1-yl. In another embodiment, R^{1c} is substituted indol-1-yl. Useful compound include those where R^{1c} is indol-1-yl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and preferably each independently selected from the group consisting of halogen, C₁₋₆ alkyl, C₁₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. In one embodiment, R^{1c} is indol-1-yl substituted with 1 or 2 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Preferably, the indol-yl group is substituted at the 3-, 4-, or 6-position or is 3,4-disubstituted.

In one embodiment, R^{1c} is unsubstituted pyrrolo[2,3-b]pyridin-1-yl. In another embodiment, R^{1c} is substituted pyrrolo[2,3-b]pyridin-1-yl. Useful compound include those where R^{1c} is pyrrolo[2,3-b]pyridin-1-yl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. In one embodiment, R^{1c} is pyrrolo[2,3-b]pyridin-1-yl substituted with 1 or 2 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Useful compounds include where the pyrrolo[2,3-b]pyridin-1-yl group is substituted at the 3-, 4-, or 6-position or is 3,4-disubstituted.

In one embodiment, R^{1c} is unsubstituted benzoimidazol-1-yl. In another embodiment, R^{1c} is substituted benzoimidazol-1-yl. Useful compound include those where R^{1c} is benzoimidazol-1-yl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. In one embodiment, R^{1c} is benzoimidazol-1-yl substituted with 1 or 2 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Useful compounds include where the benzoimidazol-1-yl group is substituted at the 4-, 5-, 6-, or 7-position, and suitably at the 4-position.

In one embodiment, R^{1c} is unsubstituted quinolinyl. In another embodiment, R^{1c} is substituted quinolinyl. In one embodiment, the quinolinyl group is quinolin-2-yl or quinolin-3-yl. Useful compounds include those where R^{1c} is quinolinyl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. In one embodiment, R^{1c} is quinolinyl substituted with 1 or 2 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Useful compounds include where the quinolinyl group is unsubstituted quinolin-2-yl or unsubstituted quinolin-3-yl, quinolin-2-yl or quinolin-3-yl substituted at the 5-, 6-, 7-, or 8-position.

In one embodiment, R^{1c} is unsubstituted pyridinyl. In another embodiment, R^{1c} is substituted pyridinyl. In one embodiment, the pyridinyl group is pyridin-2-yl, pyridin-3-yl or pyridin-4-yl. Useful compounds include those where R^{1c} is pyridinyl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. In one embodiment, R^{1c} is pyridinyl substituted with 1 or 2 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Useful compounds include where the pyridinyl group is unsubstituted pyridin-2-yl or unsubstituted pyridin-3-yl, or pyridin-2-yl or pyridin-3-yl substituted at the 5- or 6-position.

Useful compounds of the present invention include compounds of Formula I, wherein R² and R³ are each independently hydrogen or C₁₋₄ alkyl. In one embodiment, R² is hydrogen or C₁₋₄ alkyl, and R³ is hydrogen. Preferably, R² is hydrogen, methyl or ethyl, and more preferably R² is hydrogen or methyl.

In one embodiment, useful compounds of the present invention are compounds of Formula I, wherein R⁴ is optionally substituted aryl. Preferably, the aryl group is phenyl, naphthyl or biphenyl, each of which is optionally substituted. In one embodiment, R⁴ is optionally substituted phenyl. Useful compounds include those where R⁴ is phenyl substituted with 1, 2, or 3 substituents each independently selected from the group consisting of halogen, alkyl, cycloalkyl, alkoxy, alkoxyalkyl, haloalkyl, haloalkoxy, hydroxyalkyl, hydroxyalkyloxy, alkoxyalkyloxy, carboxyalkoxy, alkoxycarbonylalkoxy, aminoalkyl, hydroxy, nitro, cyano, amino, alkylamino, dialkylamino, (alkylamino)alkyl, (dialkylamino)alkyl, optionally substituted arylalkyloxy, and optionally substituted heteroarylalkyloxy, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, hydroxy(C₁₋₆)alkyloxy, C₁₋₆ alkoxy(C₁₋₆)alkyloxy, amino(C₁₋₆)alkyl, hydroxy, nitro, cyano, amino, C₁₋₆ alkylamino, di(C₁₋₄)alkylamino, C₁₋₄ alkylamino(C₁₋₄)alkyl, di(C₁₋₄)alkylamino(C₁₋₄)alkyl, phenyl(C₁₋₄)alkyloxy, and heteroaryl(C₁₋₄)alkyloxy, and more preferably halogen, C₁₋₄ alkyl, C₁₋₄ alkoxy, C₁₋₄ alkoxy(C₁₋₄)alkyl, halo(C₁₋₄)alkyl, halo(C₁₋₄)alkoxy, hydroxy(C₁₋₄)alkyl, hydroxy(C₁₋₄)alkyloxy, C₁₋₄ alkoxy(C₁₋₄)alkyloxy, amino(C₁₋₄)alkyl, hydroxy, nitro, cyano, amino, C₁₋₄ alkylamino, di(C₁₋₂)alkylamino, C₁₋₂ alkylamino(C₁₋₄)alkyl, di(C₁₋₂)alkylamino(C₁₋₄)alkyl, phenyl(C₁₋₂)alkyloxy, and heteroaryl(C₁₋₂)alkyloxy. Useful compounds include those where R⁴ is phenyl substituted with 1, 2, or 3 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, hydroxyethyloxy, hydroxypropyloxy, methoxyethyloxy, methoxypropyloxy, ethoxyethyloxy, ethoxypropyloxy, aminomethyl, hydroxy, nitro, cyano, amino, methylamino, dimethylamino, benzyloxy, pyridin-2-ylmethoxy, pyridin-3-ylmethoxy, pyridin-4-ylmethoxy, pyridazin-3-ylmethoxy, and pyrazin-2-ylmethoxy. Suitably, the phenyl group is substituted at one or more of the 2-, 3-, 4-, and 5-positions. Advantageously, R⁴ is 3,4-dimethoxyphenyl, 3-methoxy-4-hydroxypropyloxyphenyl, or 2-fluoro-3,4-dimethoxyphenyl. Suitable R⁴ groups include 3-methoxy-4-benzyloxyphenyl, 3-methoxy-4-(pyridin-3-ylmethoxy)phenyl, 3-methoxy-4-(pyridin-2-ylmethoxy)phenyl, 3-methoxy-4-(pyridin-4-ylmethoxy)phenyl, 3-methoxy-4-(pyridazin-3-ylmethoxy)phenyl, 3-methoxy-4-(pyrazin-2-ylmethoxy)phenyl, or 3-chloro-4,5-dimethoxyphenyl.

In one embodiment, useful compounds of the present invention are compounds of Formula I, wherein R⁴ is a 6-membered heteroaryl having at least one nitrogen atom, wherein the heteroaryl is optionally substituted. In another embodiment, the heteroaryl group is optionally substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. Preferably, the heteroaryl group is optionally substituted with 1, 2 or 3 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Suitable 6-membered heteroaryl groups include pyridinyl, pyrazinyl, pyridazinyl, and pyrimidinyl. In one embodiment, the 6-membered heteroaryl group is unsubstituted.

In one embodiment, useful compounds of the present invention are compounds of Formula I, wherein R⁴ is a 9-10-membered heteroaryl having at least one nitrogen atom, wherein the heteroaryl is optionally substituted. In another embodiment, the 9-10-membered heteroaryl group is optionally substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino, and preferably each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino. Preferably, the 9-10-membered heteroaryl group is optionally substituted with 1, 2 or 3 substituents each independently selected from the group consisting of chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, aminomethyl, hydroxy, nitro, cyano, amino, methylamino, and dimethylamino. Suitable 9-10-membered heteroaryl groups include quinolinyl (e.g. quinolin-6-yl), quinoxalinyl (e.g., quinoxalin-6-yl), benzothiazolyl, pyrido[3,4-b]pyridinyl, pyrido[3,2-b]pyridinyl, pyrido[4,3-b]pyridinyl, 1,5-naphthyridinyl (e.g., 1,5-naphthyridin-2-yl), and cinnolinyl (e.g., cinnolin-6-yl). In one embodiment, the 9-10-membered heteroaryl group is unsubstituted.

In one embodiment, useful compounds of the present invention are compounds of Formula I, wherein R⁴ is phenyl, wherein two adjacent carbon atoms of the phenyl ring form a bridge —O—(CH₂)ᵣ—O— or —(NR²⁶)—(CH₂)ₛ—O— to form a fused bicyclic ring; wherein r and s are 1, 2, or 3, and R²⁶ is H or C₁₋₄ alkyl, and wherein the bicyclic ring is optionally substituted. In one embodiment, R⁴ is phenyl, wherein two adjacent carbon atoms of the phenyl ring form a bridge —O—(CH₂)ᵣ—O—, wherein r is 1, 2, or 3. In one embodiment, phenyl, wherein two adjacent carbon atoms of the phenyl ring form a bridge —(NR²⁶)—(CH₂)ₛ—O— to form a fused bicyclic ring; wherein s is 1, 2, or 3, and R²⁶ is H or C₁₋₄ alkyl. Useful bicyclic rings include N-alkyl-3,4-dihydro-benzo[b][1,4]oxazin-7-yl and 3,4-dihydrobenzo[b][1,4]dioxepin-7-yl, each of which can be unsubstituted or optionally substituted by 1 or 2 substituents.

In one embodiment, compounds useful in the present invention are compounds of Formula I, having the following Formula II:

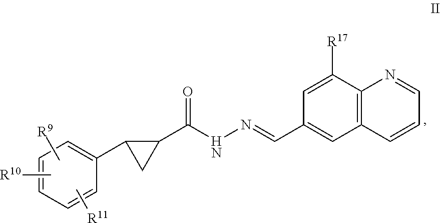

or a pharmaceutically acceptable salt or a stereoisomer thereof, wherein R⁹-R¹¹ are as defined above, and R¹⁷ is C₁₋₄ alkyl, C₁₋₄ alkoxy, C₃₋₆ cycloalkyl, halogen, halo(C₁₋₄)alkyl, or halo(C₁₋₄)alkoxy. Useful compounds of Formula II include those where R¹⁷ is methyl, ethyl, cyclopropyl, fluoro, chloro, bromo, iodo, methoxy, ethoxy, trifluoromethyl, or trifluoromethoxy.

In one embodiment, compounds useful in the present invention are compounds of Formula I, having the following Formula III:

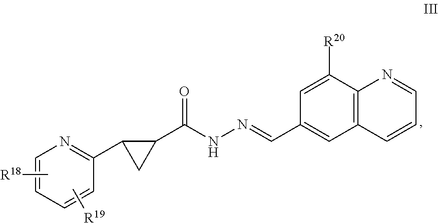

or a pharmaceutically acceptable salt or a stereoisomer thereof, wherein R¹⁸ and R¹⁹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and R²⁰ is C₁₋₄ alkyl, C₃₋₆ cycloalkyl, C₁₋₄ alkoxy, halogen, halo(C₁₋₄)alkyl, or halo(C₁₋₄)alkoxy. Useful compounds of Formula III include those where R²⁰ is methyl, ethyl, cyclopropyl, fluoro, chloro, bromo, iodo, methoxy, ethoxy, trifluoromethyl, trifluoromethoxy. Useful compounds of Formula III include those where R¹⁸ and R¹⁹ are both hydrogen, or R¹⁸ is hydrogen and R¹⁹ is selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and advantageously R¹⁹ is selected from the group consisting of C₁₋₄ alkyl, C₃₋₆ cycloalkyl, C₁₋₄ alkoxy, halogen, or halo(C₁₋₄)alkyl, and advantageously R¹⁹ is methyl, ethyl, cyclopropyl, fluoro, chloro, bromo, iodo, methoxy, ethoxy, or trifluoromethyl.

In one embodiment, compounds useful in the present invention are compounds of Formula I, having the following Formula IV:

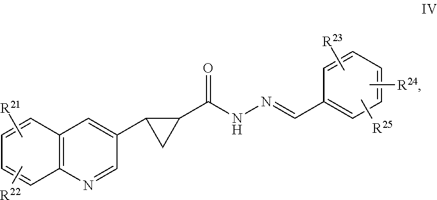

or a pharmaceutically acceptable salt or a stereoisomer thereof, wherein R²¹ and R²² are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and R²³, R²⁴, and R²⁵ are each independently selected from the group consisting of hydrogen, halogen, alkyl, cycloalkyl, alkoxy, alkoxyalkyl, haloalkyl, haloalkoxy, hydroxyalkyl, hydroxyalkyloxy, alkoxyalkyloxy, carboxyalkoxy, alkoxycarbonylalkoxy, aminoalkyl, hydroxy, nitro, cyano, amino, alkylamino, dialkylamino, (alkylamino)alkyl, (dialkylamino)alkyl, optionally substituted arylalkyloxy, and optionally substituted heteroarylalkyloxy, and preferably each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, hydroxy(C₁₋₆)alkyloxy, C₁₋₆ alkoxy(C₁₋₆)alkyloxy, amino(C₁₋₆)alkyl, hydroxy, nitro, cyano, amino, C₁₋₆ alkylamino, di(C₁₋₄)alkylamino, C₁₋₄ alkylamino(C₁₋₄)alkyl, di(C₁₋₄)alkylamino(C₁₋₄)alkyl, phenyl(C₁₋₄)alkyloxy, and heteroaryl(C₁₋₄)alkyloxy, and more preferably hydrogen, halogen, C₁₋₄ alkyl, cyclopropyl, C₁₋₄ alkoxy, C₁₋₄ alkoxy(C₁₋₄)alkyl, halo(C₁₋₄)alkyl, halo(C₁₋₄)alkoxy, hydroxy(C₁₋₄)alkyl, hydroxy(C₁₋₄)alkyloxy, C₁₋₄ alkoxy(C₁₋₄)alkyloxy, amino(C₁₋₄)alkyl, hydroxy, nitro, cyano, amino, C₁₋₄ alkylamino, di(C₁₋₂)alkylamino, C₁₋₂ alkylamino(C₁₋₄)alkyl, di(C₁₋₂)alkylamino(C₁₋₄)alkyl, phenyl(C₁₋₂)alkyloxy, and heteroaryl(C₁₋₂)alkyloxy.

Useful compounds of Formula IV include those where R²³, R²⁴, and R²⁵ are each independently selected from the group consisting of hydrogen, chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, hydroxyethyloxy, hydroxypropyloxy, methoxyethyloxy, methoxypropyloxy, ethoxyethyloxy, ethoxypropyloxy, aminomethyl, hydroxy, nitro, cyano, amino, methylamino, dimethylamino, benzyloxy, pyridin-2-ylmethoxy, pyridin-3-ylmethoxy, pyridin-4-ylmethoxy, pyridazin-3-ylmethoxy, and pyrazin-2-ylmethoxy. Suitably, R²³, R²⁴, and R²⁵ are each independently at the 2-, 3-, 4-, and 5-positions. Advantageously, R²³, R²⁴, and R²⁵ are such that the phenyl group is substituted as follows: 3,4-dimethoxy, 3-methoxy-4-hydroxypropyloxy, 2-fluoro-3,4-dimethoxy, 3-methoxy-4-benzyloxy, 3-methoxy-4-(pyridin-3-ylmethoxy), 3-methoxy-4-(pyridin-2-ylmethoxy), 3-methoxy-4-(pyridin-4-ylmethoxy), 3-methoxy-4-(pyridazin-3-ylmethoxy), 3-methoxy-4-(pyrazin-2-ylmethoxy), or 3-chloro-4,5-dimethoxy.

In one embodiment, compounds useful in the present invention are compounds of Formula I, having the following Formula V:

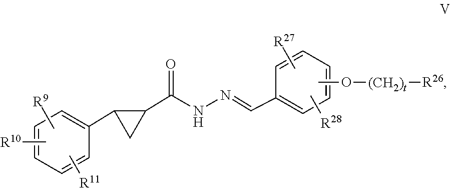

or a pharmaceutically acceptable salt or a stereoisomer thereof, wherein R⁹-R¹¹ are as defined above, R²⁶ is optionally substituted phenyl or optionally substituted heteroaryl, R²⁷ and R²⁸ each independently selected from the group consisting of hydrogen, halogen, alkyl, cycloalkyl, alkoxy, alkoxyalkyl, haloalkyl, haloalkoxy, hydroxyalkyl, hydroxyalkyloxy, alkoxyalkyloxy, carboxyalkoxy, alkoxycarbonylalkoxy, aminoalkyl, hydroxy, nitro, cyano, amino, alkylamino, dialkylamino, (alkylamino)alkyl, and (dialkylamino)alkyl, and t is 1, 2, or 3. Useful compounds of Formula V include those where t is 1. Useful compounds of Formula V include those where R²⁷ and R²⁸ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, hydroxy(C₁₋₆)alkyloxy, C₁₋₆ alkoxy(C₁₋₆)alkyloxy, amino(C₁₋₆)alkyl, hydroxy, nitro, cyano, amino, C₁₋₆ alkylamino, di(C₁₋₄)alkylamino, C₁₋₄ alkylamino(C₁₋₄)alkyl, and di(C₁₋₄)alkylamino(C₁₋₄)alkyl, and more preferably hydrogen, halogen, C₁₋₄ alkyl, cyclopropyl, C₁₋₄ alkoxy, C₁₋₄ alkoxy(C₁₋₄)alkyl, halo(C₁₋₄)alkyl, halo(C₁₋₄)alkoxy, hydroxy(C₁₋₄)alkyl, hydroxy(C₁₋₄)alkyloxy, C₁₋₄ alkoxy(C₁₋₄)alkyloxy, amino(C₁₋₄)alkyl, hydroxy, nitro, cyano, amino, C₁₋₄ alkylamino, di(C₁₋₂)alkylamino, C₁₋₂ alkylamino(C₁₋₄)alkyl, and di(C₁₋₂)alkylamino(C₁₋₄)alkyl. Useful compounds of Formula V include those where R²⁷ and R²⁸ are each independently selected from the group consisting of hydrogen, chloro, bromo, fluoro, methyl, ethyl, n-propyl, 2-propyl, cyclopropyl, methoxy, ethoxy, methoxymethyl, methoxyethyl, trifluoromethyl, trifluoroethyl, trifluoromethoxy, trifluoroethoxy, hydroxymethyl, hydroxyethyl, hydroxyethyloxy, hydroxypropyloxy, methoxyethyloxy, methoxypropyloxy, ethoxyethyloxy, ethoxypropyloxy, aminomethyl, hydroxy, nitro, cyano, amino, methylamino, and dimethylamino. In one embodiment, R²⁷ and R²⁸ are both hydrogen.

In one embodiment, —O—(CH₂)—R²⁶ in Formula V is attached at the 4-position of the phenyl ring.

In one embodiment, R²⁶ is optionally substituted phenyl. Useful optional substituents for the phenyl group in R²⁶ are those defined for optionally substituted phenyl for R⁴ above. In one embodiment, R²⁶ is optionally substituted heteroaryl. Useful heteroaryl groups include 6-membered heteroaryl groups having at least on nitrogen atom, such as pyridinyl, pyridazinyl, and pyrazinyl. Useful optional substituents for heteroaryl groups in R²⁶ are those defined for optionally substituted heteroaryl in R⁴. Advantageously, R²⁶ is optionally substituted pyridinyl.

The groups R⁶, R⁷, R⁹, R¹⁰, R¹¹, and R¹⁷-R²⁸, when they are not equal to hydrogen, take the place of a hydrogen atom that would otherwise be present in any position on the phenyl ring to which the particular R group is attached. Similarly, optional substituents attached to cycloalkyl, aryl, and heteroaryl rings each take the place of a hydrogen atom that would otherwise be present in any position of the cycloalkyl, aryl or heteroaryl rings.

Preferably, R² and R³ are each independently hydrogen or C₁₋₆ alkyl, more preferably hydrogen or C₁₋₄ alkyl, and more preferably hydrogen, methyl or ethyl. Useful compounds include those where R² is hydrogen or C₁₋₆ alkyl and R³ is hydrogen. Useful compounds include those where R² and R³ both are hydrogen.

Exemplary preferred compounds useful in the present invention include:
2-(3-chlorophenylthio)-N′-(3,4-dimethoxybenzylidene)propane-hydrazide;
N′-(3,4-dimethoxybenzylidene)-3-(1-methyl-1H-indol-3-yl)propane-hydrazide;
3-(3-methyl-1H-indol-1yl)-N′-((8-methylquinolin-6-yl)methylene)-propanehydrazide;
N′-(4-(3-hydroxypropoxy)-3-methoxybenzylidene)-3-(3-methyl-1H-indol-yl)propanehydrazide;
N-(4-chlorophenethyl)-2-(3,4-dimethoxybenzylidene)hydrazine-carboxamide;
N′-(3,4-dimethoxybenzylidene)-2-(3-trifluoromethyl)phenyl)-cyclopropanecarbohydrazide;
N′-(3,4-dimethoxybenzylidene)-3-(3,4-dimethyl-1H-indol-1-yl)-propanehydrazide;
3-(3,4-dimethyl-1H-indol-1-yl)-N′-(2-fluoro-4,5-dimethoxy-benzylidene)propanehydrazide;
N′-(3,4-dimethoxybenzylidene)-N-methyl-2-phenylcyclopropane-carbohydrazide;

and the pharmaceutically acceptable salts and stereoisomers thereof.

Further examplary preferred compounds useful in the present invention include:
trans-N′-(3,4-dimethoxybenzylidene)-2-(3-trifluoromethyl)phenyl)-cyclopropanecarbohydrazide;
trans-N′-(3,4-dimethoxybenzylidene)-N-methyl-2-phenylcyclopropane-carbohydrazide;

and the pharmaceutically acceptable salts thereof.

Further examplary preferred compounds useful in the present invention include:
N′-((8-methylquinolin-6-yl)methylene)-2-phenylcyclopropane-carbohydrazide;
N′-(4-(benzyloxy)-3-methoxybenzylidene)-3-(3,4-dimethyl-1H-indol-1-yl)propanehydrazide;
3-(3,4-dimethyl-1H-indol-1-yl)-N′-(3-methoxy-4-(pyridin-2-ylmethoxy)benzylidene)propanehydrazide;
N′-(3,4-dimethoxybenzylidene)-3-(3-methyl-1H-pyrrolo[2,3-b]pyridin-1-yl)propanehydrazide;
N′-(3-chloro-4,5-dimethoxybenzylidene)-3-(3,4-dimethyl-1H-indol-1-yl)propanehydrazide;
3-(3,4-dimethyl-1H-indol-1-yl)-N′-(3-methoxy-4-(pyridin-3-ylmethoxy)benzylidene)propanehydrazide;
N′-(3,4-dimethoxybenzylidene)-3-(4-methyl-1H-benzo[d]imidazol-1-yl)propanehydrazide;
3-(3,4-dimethyl-1H-indol-1-yl)-N′-(3-methoxy-4-(pyridin-4-ylmethoxy)benzylidene)propanehydrazide;
3-(3,4-dimethyl-1H-indol-1-yl)-N′-(3-methoxy-4-(pyridazin-3-ylmethoxy)benzylidene)propanehydrazide;
N′-(3-methoxy-4-(pyridin-3-ylmethoxy)benzylidene)-2-phenylcyclopropanecarbohydrazide;
3-(3,4-dimethyl-1H-indol-1-yl)-N′-(3-methoxy-4-(pyrazin-2-ylmethoxy)benzylidene)propanehydrazide;
N′-((8-methoxyquinolin-6-yl)methylene)-2-phenylcyclopropane-carbohydrazide;
3-(1-methyl-1H-indol-3-yl)-N′-((8-methylquinolin-6-yl)methylene)-propanehydrazide;
2-phenyl-N′-((8-(trifluoromethyl)quinolin-6-yl)methylene)cyclopropanecarbohydrazide
N′-((8-chloroquinolin-6-yl)methylene)-2-phenylcyclopropane-carbohydrazide;
3-(6-methoxypyridin-2-yl)-N′-((8-methylquinolin-6-yl)methylene)propanehydrazide;
3-(3,4-dimethyl-1H-indol-1-yl)-N′-((8-methoxyquinolin-6-yl)methylene)propanehydrazide;
2-(3-chlorophenyl)-N′-((8-chloroquinolin-6-yl)methylene)-cyclopropanecarbohydrazide;
2-(3-chlorophenyl)-N′-((8-methylquinolin-6-yl)methylene)-cyclopropanecarbohydrazide;
2-(3-chlorophenyl)-N′-((8-methoxyquinolin-6-yl)methylene)-cyclopropanecarbohydrazide;
N′-((1,5-naphthyridin-2-yl)methylene)-2-(3-chlorophenyl)-cyclopropanecarbohydrazide;
3-(3-methyl-1H-indol-1-yl)-N′-(quinoxalin-6-ylmethylene)-propanehydrazide;
N′-((1,5-naphthyridin-2-yl)methylene)-3-(3-methyl-1H-indol-1-yl)propanehydrazide;
N′-(cinnolin-6-ylmethylene)-3-(3-methyl-1H-indol-1-yl)propanehydrazide;
2-(3-chlorophenylthio)-N′-((8-methylquinolin-6-yl)methylene)-acetohydrazide;
N′-(3,4-dimethoxybenzylidene)-2-(quinolin-2-yl)cyclopropane-carbohydrazide;
N′-(3,4-dimethoxybenzylidene)-2-(6-methylpyridin-2-yl)cyclopropanecarbohydrazide;
N′-(3,4-dimethoxybenzylidene)-2-(quinolin-3-yl)cyclopropane-carbohydrazide;
N′-(3,4-dimethoxybenzylidene)-3-(quinolin-3-yl)propanehydrazide;
2-(4-fluorophenyl)-N′-((8-methylquinolin-6-yl)methylene)-cyclopropanecarbohydrazide;
2-(6-methoxypyridin-3-yl)-N′-((8-methylquinolin-6-yl)methylene)-cyclopropanecarbohydrazide;
2-(6-methoxypyridin-2-yl)-N′-((8-methylquinolin-6-yl)methylene)cyclopropanecarbohydrazide;
2-(5-chloropyridin-2-yl)-N′-((8-methylquinolin-6-yl)methylene)-cyclopropanecarbohydrazide;
N′-((8-fluoroquinolin-6-yl)methylene)-2-phenylcyclopropane-carbohydrazide;
N′-((3,4-dihydro-2H-benzo[b][1,4]dioxepin-7-yl)methylene)-3-(3,4-dimethyl-1H-indol-1-yl)propanehydrazide;
N′-(3,4-dimethoxybenzylidene)-3-(4-methoxy-3-methyl-1H-indol-1-yl)propanehydrazide;
3-(4-chloro-3-((dimethylamino)methyl)-1H-indol-1-yl)-N′-(3,4-dimethoxybenzylidene)propanehydrazide;
N′-(3,4-dimethoxybenzylidene)-3-(1,7-dimethyl-1H-indol-3-yl)propanehydrazide;
3-(3-chloro-4-methyl-1H-indol-1-yl)-N′-(3,4-dimethoxybenzylidene)-propanehydrazide;
N′-(3,4-dimethoxybenzylidene)-3-(4-(hydroxymethyl)-3-methyl-1H-indol-1-yl)propanehydrazide;
2-(3-chlorophenylsulfonyl)-N′-(3,4-dimethoxybenzylidene)-propanehydrazide;
N′-((2-methoxy-4-methylquinolin-6-yl)methylene)-3-(3-methyl-1H-indol-1-yl)propanehydrazide;
2-(3-chlorophenylthio)-N′-((4,5-dimethoxypyridin-2-yl)methylene)propanehydrazide;
N′-(3,4-dimethoxybenzylidene)-3-(1H-indol-1-yl)propanehydrazide;
N′-(3,4-dimethoxybenzylidene)-2-(phenylthio)acetohydrazide;
N′-(3,4-dimethoxybenzylidene)-3-(3-methyl-1H-indol-1-yl)propanehydrazide;
N′-(3,4-dimethoxybenzylidene)-3-(4-methyl-1H-indol-1-yl)propanehydrazide;
N′-((3,4-dihydro-2H-benzo[b][1,4]di-oxepin-7-yl)methylene)-3-(3-methyl-1H-indol-1-yl)propanehydrazide;
3-(3-methyl-1H-indol-1-yl)-N′-(quinolin-6-ylmethylene)-propanehydrazide;
3-(3-methyl-1H-indol-1-yl)-N′-((4-methyl-3,4-dihydro-2H-benzo[b][1,4]-oxazin-7-yl)methylene)propanehydrazide;
3-(3-methyl-1H-indol-1-yl)-N′-((3-methylquinolin-6-yl)methylene)-propanehydrazide;
3-(3-methyl-1H-indol-1-yl)-N′-((4-methylquinolin-6-yl)methylene)-propanehydrazide;
N′-((4-methoxyquinolin-6-yl)methylene)-3-(3-methyl-1H-indol-1-yl)propanehydrazide;
3-(3,4-dimethyl-1H-indol-1-yl)-N′-((8-methylquinolin-6-yl)-methylene)propanehydrazide;
2-(3,4-dimethoxybenzylidene)-N-phenethylhydrazinecarboxamide;
2-(3-chlorophenylthio)-N′-(3,4-dimethoxybenzylidene)acetohydrazide;
N′-(3,4-dimethoxybenzylidene)-2-(3-(hydroxymethyl)phenylthio)-acetohydrazide;
3-(3-methyl-1H-indol-1-yl)-N′-(quinolin-7-ylmethylene)-propanehydrazide;
3-(3-methyl-1H-indol-1-yl)-N′-((2-methylquinolin-6-yl)methylene)-propanehydrazide;

and the pharmaceutically acceptable salts and stereoisomers thereof.

Further examplary preferred compounds useful in the present invention include:
trans-N′-((8-methylquinolin-6-yl)methylene)-2-phenylcyclopropane-carbohydrazide;
trans-N′-((8-methylquinolin-6-yl)methylene)-2-phenylcyclopropane-carbohydrazide, E1 enantiomer;
trans-N′-((8-methylquinolin-6-yl)methylene)-2-phenylcyclopropane-carbohydrazide, E2 enantiomer;
trans-N′-(3-methoxy-4-(pyridin-3-ylmethoxy)benzylidene)-2-phenylcyclopropanecarbohydrazide;
trans-N′-((8-methoxyquinolin-6-yl)methylene)-2-phenylcyclopropanecarbohydrazide;
trans-2-phenyl-N′-((8-(trifluoromethyl)quinolin-6-yl)methylene)cyclopropanecarbohydrazide;
trans-N′-((8-chloroquinolin-6-yl)methylene)-2-phenylcyclopropane-carbohydrazide;
trans-2-(3-chlorophenyl)-N′-((8-chloroquinolin-6-yl)methylene)-cyclopropanecarbohydrazide;
trans-2-(3-chlorophenyl)-N′-((8-methylquinolin-6-yl)methylene)-cyclopropanecarbohydrazide;
trans-2-(3-chlorophenyl)-N′-((8-methoxyquinolin-6-yl)methylene)cyclopropanecarbohydrazide;
trans-N′-((1,5-naphthyridin-2-yl)methylene)-2-(3-chlorophenyl)cyclopropanecarbohydrazide;
trans-N′-(3,4-dimethoxybenzylidene)-2-(quinolin-2-yl)cyclopropanecarbohydrazide;
trans-N′-(3,4-dimethoxybenzylidene)-2-(6-methylpyridin-2-yl)cyclopropanecarbohydrazide;
trans-N′-(3,4-dimethoxybenzylidene)-2-(quinolin-3-yl)cyclopropanecarbohydrazide;
trans-2-(4-fluorophenyl)-N′-((8-methylquinolin-6-yl)methylene)cyclopropanecarbohydrazide;
trans-2-(6-methoxypyridin-3-yl)-N′-((8-methylquinolin-6-yl)methylene)cyclopropanecarbohydrazide;
trans-2-(6-methoxypyridin-2-yl)-N′-((8-methylquinolin-6-yl)methylene)cyclopropanecarbohydrazide;
trans-2-(5-chloropyridin-2-yl)-N′-((8-methylquinolin-6-yl)methylene)cyclopropanecarbohydrazide;
trans-N′-((8-fluoroquinolin-6-yl)methylene)-2-phenylcyclopropanecarbohydrazide;

and the pharmaceutically acceptable salts thereof.

Useful cycloalkyl groups are selected from C₃₋₈ cycloalkyl, more preferably from C₃₋₆ cycloalkyl, and more preferably from C₃₋₅ cycloalkyl. Typical cycloalkyl groups include cyclopropyl, cyclobutyl, cyclopentyl, cyclohexyl, cycloheptyl, and cyclooctyl.

Useful halo or halogen groups include fluorine, chlorine, bromine and iodine.

Useful alkyl groups are selected from straight-chained and branched C₁₋₁₀ alkyl groups, more preferably straight chain C₁₋₆ alkyl groups and branched chain C₁₋₆ alkyl groups, and more preferably straight chain C₁₋₄ alkyl groups and branched chain C₁₋₄ alkyl groups. Typical C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl, among others.

Useful haloalkyl groups include any of the above-mentioned C₁₋₁₀ alkyl groups substituted by one or more fluorine, chlorine, bromine or iodine atoms (e.g., fluoromethyl, difluoromethyl, trifluoromethyl, pentafluoroethyl, 1,1-difluoroethyl and trichloromethyl groups).

Useful hydroxyalkyl groups include any of the above-mentioned C₁₋₁₀ alkyl groups substituted by one or more hydroxy groups (e.g., hydroxymethyl, hydroxyethyl, hydroxypropyl and hydroxybutyl groups, and especially hydroxymethyl, 1-hydroxyethyl, 2-hydroxypropyl, and 3-hydroxybutyl).

Useful alkoxy or alkyloxy groups include oxygen substituted by one of the C₁₋₁₀ alkyl groups mentioned above (e.g., methoxy, ethoxy, propoxy, iso-propoxy, butoxy, tert-butoxy, iso-butoxy, sec-butoxy, and pentyloxy).

Useful alkoxyalkyl groups include any of the above-mentioned C₁₋₁₀ alkyl groups substituted with any of the above-mentioned alkoxy groups (e.g., methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl).

Useful haloalkoxy groups include oxygen substituted by one of the C₁₋₁₀ haloalkyl groups mentioned above (e.g., fluoromethoxy, difluoromethoxy, and trifluoromethoxy).

Useful aryl groups are C₆₋₁₄ aryl, especially C₆₋₁₀ aryl. Typical C₆₋₁₄ aryl groups include phenyl, naphthyl, phenanthryl, anthracyl, indenyl, isoindenyl, azulenyl, biphenyl, biphenylenyl, and fluorenyl groups, more preferably phenyl, naphthyl, and biphenyl groups.

Useful arylalkyl groups include any of the above-mentioned C₁₋₁₀ alkyl groups substituted with any of the above-mentioned aryl groups (e.g., benzyl, phenylethyl, and phenylpropyl).

The term “heteroaryl” as employed herein refers to groups having 5 to 14 ring atoms, with 6, 10 or 14 π electrons shared in a cyclic array, and containing carbon atoms and 1, 2, or 3 heteroatoms independently chosen from oxygen, nitrogen and sulfur. Examples of heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. Preferred heteroaryl groups include pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, pyrimidin-5-yl, and pyrimidin-6-yl), indolyl (e.g., indol-3-yl and indol-1-yl), quinolinyl (e.g., quinolin-2-yl), pyrazinyl (e.g., pyrazin-2-yl and pyrazin-3-yl), quinoxalinyl, benzothiazolyl, pyrido[3,4-b]pyridinyl, pyrido[3,2-b]pyridinyl, and pyrido[4,3-b]pyridinyl.

Useful heteroarylalkyl groups include any of the above-mentioned C₁₋₁₀ alkyl groups substituted with any of the above-mentioned heteroaryl groups (e.g., pyridin-2-ylmethyl, pyridin-3-ylmethyl, and pyridin-4-ylmethyl).

Useful arylalkyloxy or arylalkoxy groups include oxygen substituted by one of the arylalkyl groups mentioned above (e.g., benzyloxy, phenylethoxy, and phenylpropoxy).

Useful heteroarylalkyloxy or heteroarylalkoxy groups include oxygen substituted with any of the above-mentioned heteroarylalkyl groups (e.g., pyridin-2-ylmethoxy, pyridin-3-ylmethoxy, and pyridin-4-ylmethoxy).

As used herein, the term “amino” or “amino group” refers to —NH₂.

Useful aminoalkyl groups include any of the above-mentioned C₁₋₁₀ alkyl groups substituted with an amino group.

Useful alkylamino and dialkylamino groups are —NHR¹⁵ and —NR¹⁵R¹⁶, respectively, wherein R¹⁵ and R¹⁶ are each independently selected from a C₁₋₁₀ alkyl group.

Useful alkylaminoalkyl and dialkylaminoalkyl groups are any of the above-mentioned C₁₋₁₀ alkyl groups substituted by any of the above-mentioned alkylamino and dialkylamino groups, respectively.

As used herein, the term “carboxy” refers to —COOH.

Useful carboxyalkyl groups include any of the above-mentioned C₁₋₁₀ alkyl groups substituted by —COOH.

Useful hydroxyalkyloxy groups include oxygen substituted by one of the C₁₋₁₀ hydroxyalkyl groups mentioned above (e.g., hydroxymethyloxy, hydroxyethyloxy, hydroxypropyloxy, and hydroxybutyloxy groups).

Useful alkoxyalkyloxy groups include oxygen substituted by one of the C₁₋₁₀ alkoxyalkyl groups mentioned above (e.g., methoxymethyloxy, methoxyethyloxy, methoxypropyloxy, methoxybutyloxy, ethoxymethyloxy, ethoxyethyloxy, and ethoxypropyloxy groups).

Useful carboxyalkoxy groups include any of the above-mentioned C₁₋₁₀ alkoxy groups substituted by —COOH.

The term “carbonyl” means —C(O)—.

Useful alkoxycarbonyl groups include a carbonyl group substituted with any of the above-mentioned C₁₋₁₀ alkoxy groups (e.g., methoxycarbonyl, ethoxycarbonyl and propoxycarbonyl groups).

Useful alkoxycarbonylalkoxy groups include any of the above-mentioned C₁₋₁₀ alkoxy groups substituted by an alkoxycarbonyl group (e.g., methoxycarbonylmethoxy, methoxycarbonylethoxy, and methoxycarbonylpropoxy groups).

The term “oxy” means an oxygen (O) atom.

As used herein, the term “optionally substituted” refers to a group that may be unsubstituted or substituted.

Generally and unless defined otherwise, the phrase “optionally substituted” used herein refers to a group or groups being optionally substituted with one or more, preferably 1, 2 or 3, substituents independently selected from the group consisting of amino, hydroxy, nitro, halogen, cyano, thiol, C₁₋₆ alkyl, C₂₋₆ alkenyl, C₂₋₆ alkynyl, C₃₋₆ cycloalkyl, C₃₋₆ cycloalkenyl, C₃₋₆ cycloheteralkyl, C₃₋₆ cycloheteroalkenyl, C₆₋₁₀ aryl, 5-10 membered heteroaryl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₆₋₁₀ aryl(C₁₋₆)alkyl, C₆₋₁₀ aryl(C₂₋₆)alkenyl, C₆₋₁₀ aryl(C₁₋₆)alkoxy, C₁₋₆ aminoalkyl, C₁₋₆ aminoalkoxy, C₁₋₆ hydroxyalkyl, C₂₋₆ hydroxyalkoxy, benzamido, mono(C₁₋₄)alkylamino, di(C₁₋₄)alkylamino, mono(C₁₋₄)alkylamino(C₁₋₄)alkyl, di(C₁₋₄)alkylamino(C₁₋₄)alkyl, hetero aryl(C₁₋₄)alkyloxy, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy C₂₋₁₀ mono(carboxyalkyl)amino, bis(C₂₋₁₀ carboxyalkyl)amino, aminocarbonyl, C₆₋₁₄ aryl(C₁₋₆)alkoxycarbonyl, C₂₋₆ alkynylcarbonyl, C₁₋₆ alkylsulfonyl, C₂₋₆ alkynylsulfonyl, C₆₋₁₀ arylsulfonyl, C₆₋₁₀ aryl(C₁₋₆)alkylsulfonyl, C₁₋₆ alkylsulfinyl, C₁₋₆ alkylsulfonamido, C₆₋₁₀ arylsulfonamido, C₆₋₁₀ aryl(C₁₋₆) alkylsulfonamido, C₁₋₆ alkyliminoamino, formyliminoamino, C₂₋₆ carboxyalkoxy, C₂₋₆ carboxyalkyl, and carboxy(C₁₋₆)alkylamino. Useful optional substituents include halo, halo(C₁₋₆)alkyl, aryl, heterocycle, cycloalkyl, C₁₋₆ alkyl, C₂₋₆ alkenyl, C₂₋₆ alkynyl, aryl(C₁₋₆)alkyl, aryl(C₂₋₆)alkenyl, aryl(C₂₋₆)alkynyl, cycloalkyl(C₁₋₆)alkyl, heterocyclo(C₁₋₆)alkyl, hydroxy(C₁₋₆)alkyl, amino(C₁₋₆)alkyl, carboxy(C₁₋₆)alkyl, alkoxy(C₁₋₆)alkyl, nitro, amino, ureido, cyano, alkylcarbonylamino, hydroxy, thiol, alkylcarbonyloxy, azido, alkoxy, carboxy, aminocarbonyl, and C₁₋₆ alkylthiol groups mentioned above. Preferred optional substituents include halo, halo(C₁₋₆)alkyl, halo(C₁₋₆)alkoxy, hydroxy(C₁₋₆)alkyl, amino(C₁₋₆)alkyl, hydroxy, nitro, C₁₋₆ alkyl, cyclopropyl, C₁₋₆ alkoxy, and amino.

The methods of the present invention also include the use of a pharmaceutically acceptable salt of a compound according to any of Formulae I-V. The term pharmaceutically acceptable salt refers to an acid- and/or base-addition salt of a compound according to any of Formulae I-V. Acid-addition salts can be formed by adding an appropriate acid to the compound according to any of Formulae I-V. Base-addition salts can be formed by adding an appropriate base to the compound of any of Formulae I-V. Said acid or base does not substantially degrade, decompose, or destroy said compound of any of Formulae I-V. Examples of suitable pharmaceutically acceptable salts include hydrochloride, hydrobromide, acetate, furmate, maleate, oxalate, and succinate salts. Other suitable salts include sodium, potassium, carbonate, and tromethamine salts.

It is also to be understood that the present invention is considered to encompass the use of stereoisomers as well as optical isomers, e.g., mixtures of enantiomers as well as individual enantiomers and diastereomers, which arise as a consequence of structural asymmetry in selected compounds of the present series. It is further understood that the present invention encompasses the use of tautomers of a compound of any of Formulae I-V. Tautomers are well-known in the art and include keto-enol tautomers.

It is also understood that the compounds of any of Formulae I-V include both the E and Z isomers, in varying ratios, of the hydrazone. As is known in the art, the hydrazone moiety can isomerize between the E and Z isomers, as shown in the following schematic:

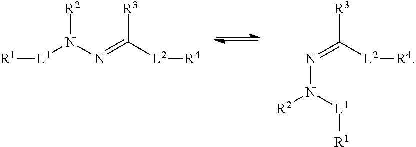

Some of the compounds disclosed herein may contain one or more asymmetric centers and may thus give rise to enantiomers, diastereomers, and other stereoisomeric forms. The present invention is meant to encompass the uses of all such possible forms, as well as their racemic and resolved forms and mixtures thereof. The individual enantiomers may be separated according to methods known to those of ordinary skill in the art in view of the present disclosure. When the compounds described herein contain olefinic double bonds or other centers of geometric asymmetry, and unless specified otherwise, it is intended that they include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not minor images of one another (diastereomers).

The term “chiral center” refers to a carbon atom to which four different groups are attached.

The terms “enantiomer” and “enantiomeric” refer to a molecule that cannot be superimposed on its minor image and hence is optically active wherein the enantiomer rotates the plane of polarized light in one direction and its minor image compound rotates the plane of polarized light in the opposite direction.

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

The compounds of any of Formulae I-V may also be solvated, including hydrated. Hydration may occur during manufacturing of the compounds or compositions comprising the compounds, or the hydration may occur over time due to the hygroscopic nature of the compounds.

When any variable occurs more than one time in any constituent or in Formula I-V, its definition on each occurrence is independent of its definition at every other occurrence, unless otherwise indicated. Also, combinations of substituents and/or variables are permissible only if such combinations result in stable compounds.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or alkyl, or together with the nitrogen to which they are bound, form a saturated or unsaturated 5-, 6-, or 7-membered ring.

Although detailed definitions have not been provided for every term used above, each term is understood by one of ordinary skill in the art.

Compounds of any of Formulae I-V, and pharmaceutically acceptable salts and stereoisomers thereof, can be used to inhibit a taste modulating protein. Such inhibition may be in vitro or in vivo. The amount of the compound of the present invention used to inhibit the taste modulating protein may not necessarily be the same when used in vivo compared to in vitro. Factors such as pharmacokinetics and pharmacodynamics of the particular compound may require that a larger or smaller amount of the compound of the present invention be used when inhibiting a taste modulating protein in vivo. Accordingly, in one embodiment, the present invention is directed to a method of inhibiting a taste, comprising contacting the taste modulating protein with a compound of any of Formulae I-V, or a pharmaceutically acceptable salt or stereoisomer thereof. In one embodiment, the method comprises contacting a cell with a compound of any of Formulae I-V, or a pharmaceutically acceptable salt or stereoisomer thereof, wherein said cell expresses said taste modulating protein. In another embodiment, the method comprises administering a compound of any of Formulae I-V, or a pharmaceutically acceptable salt or stereoisomer thereof to a subject in an amount sufficient to inhibit a taste modulating protein, wherein said subject has or expresses said taste modulating protein. Furthermore, when administered orally, the compound may be dispersed or diluted by saliva.

By way of example, the present invention is directed to a method of inhibiting a taste modulating protein, comprising contacting said protein with a compound of any of Formulae I-V, or a pharmaceutically acceptable salt or stereoisomer thereof, and inhibiting the protein by at least about 10%, 20%, 30%, 40%, 50%, 60%, 70%, 80%, 90%, or 95%, or from about 50% to about 99%, and preferably by about 10% to about 50%. In one aspect of said embodiment, said taste modulating protein is a naturally occurring taste modulating protein. In another aspect of said embodiment, said taste modulating protein is a naturally occurring human taste modulating protein.

Any amount of the compound of any of Formulae I-V that provides the desired degree of inhibition can be used. For example, a compound of Formula I may be used at a concentration of about 0.1 μM to about 1,000 μM to inhibit a taste modulating protein. Alternatively, concentrations of about 1, 10 or 100 μM of a compound of any of Formulae I-V may be used to inhibit a taste modulating protein. In certain embodiments, a single dose or two to four divided daily doses, provided on a basis of about 0.001 to 100 mg per kilogram of body weight per day, preferably about 0.01 to about 25 mg/kg of body weight per day is appropriate. The substance is preferably administered orally, but parenteral routes such as the subcutaneous, intramuscular, intravenous or intraperitoneal routes or any other suitable delivery system, such as intranasal or transdermal routes can also be employed.

As used herein, the term “inhibiting” and grammatical variants thereof refers to interfering with the normal activity of. For example, inhibiting a taste modulating protein means interfering with the normal activity of a taste modulating protein. Inhibiting includes but is not necessarily limited to modulating, modifying, inactivating, and the like.

As used herein, the phrase “taste modulating protein” refers to a TRPM5 protein, and includes naturally and recombinantly produced TRPM5 proteins; natural, synthetic, and recombinant biologically active polypeptide fragments of said protein; biologically active polypeptide variants of said protein or fragments thereof, including hybrid fusion proteins, dimers, and higher multimers, such as tetramers; biologically active polypeptide analogs of said protein or fragments or variants thereof, including cysteine substituted analogs. The taste modulating protein may be a nonhuman protein, for example a nonhuman mammalian protein, or in other embodiments a nonhuman protein such as but not limited to a cow, horse, sheep, pig, chicken, turkey, quail, cat, dog, mouse, rat, rabbit, monkey, or guinea pig taste modulating protein. The taste modulating protein may be generated and/or isolated by any means known in the art. An example of the taste modulating protein and methods of producing the protein are disclosed in, for example, Liu and Liman, Proc. Nat'l Acad. Sci. USA 100: 15160-15165 (2003); D. Prawitt, et al., Proc. Nat'l Acad. Sci. USA 100:15166-71 (2003); and Ulrich, N. D., et al., Cell Calcium 37: 267-278 (2005); each of which is fully incorporated by reference herein.

A homologue is a protein that may include one or more amino acid substitutions, deletions, or additions, either from natural mutations of human manipulation. Thus, by way of example, a taste modulating protein may include one or more amino acid substitutions, deletions or additions, either from natural mutations or human manipulation. As indicated, changes are preferably of a minor nature, such as conservative amino acid substitutions that do not significantly affect the folding or activity of the protein.

The variant taste modulating proteins which may be inhibited in accordance with the present invention comprise non-conservative modifications (e.g., substitutions). By “nonconservative” modification herein is meant a modification in which the wild-type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. In one embodiment, the variant taste modulating proteins used in accordance with the present invention have at least one nonconservative modification.

In one embodiment, the taste modulating protein that is a nonhuman protein, such as, but not limited to, a cow, horse, sheep, pig, chicken, turkey, quail, cat, dog, mouse, rat, rabbit, monkey, or guinea pig taste modulating protein.

In one embodiment, the present invention is directed to a method of inhibiting the depolarization of a taste receptor cell, comprising contacting the taste receptor cell with a compound of any of Formulae I-V, or a pharmaceutically acceptable salt or stereoisomer thereof. For example, a compound of any of Formulae I-V may inhibit the depolarization of a taste receptor cell by a mechanism other than, or in addition to, the mechanism of inhibiting a taste receptor protein. In one aspect of this embodiment the present invention, the method comprises contacting a taste receptor cell with a compound of any of Formulae I-V, or a pharmaceutically acceptable salt or stereoisomer thereof, wherein said taste receptor cell can detect a sweet, bitter, sour, salty, or umami taste. In another aspect of this embodiment, the method comprises administering a compound of any of Formulae I-V, or a pharmaceutically acceptable salt or stereoisomer thereof, in an amount sufficient to inhibit the depolarization of a taste receptor cell. Furthermore, when administered orally, the compound may be dispersed or diluted by saliva.

By way of example, the present invention is directed to a method of inhibiting the depolarization of a taste receptor cell, comprising contacting said taste receptor cell with a compound of any of Formulae I-V, or a pharmaceutically acceptable salt or stereoisomer thereof, and inhibiting the depolarization of the taste receptor cell by at least about 10%, 20%, 30%, 40%, 50%, 60%, 70%, 80%, 90%, or 95%, or from about 60% to about 99%, or alternatively from about 30% to about 75%. In one aspect of said embodiment, the present invention is directed to a method of inhibiting the depolarization of a taste receptor cell, comprising contacting said protein with a compound of any of Formulae I-V, or a pharmaceutically acceptable salt or stereoisomer thereof, and inhibiting the depolarization of the taste receptor cell by at least about 10%, 20%, 30%, 40%, 50%, 60%, 70%, 80%, 90%, or 95%, or from about 50% to about 99%, or alternatively from about 20% to about 60%, and wherein said taste receptor cell is a naturally occurring taste modulating protein. In another aspect of said embodiment, the present invention is directed to a method of inhibiting a taste receptor cell, comprising contacting said protein with a compound of any of Formulae I-V, or a pharmaceutically acceptable salt or stereoisomer thereof, and inhibiting the taste receptor cell by at least about 10%, 20%, 30%, 40%, 50%, 60%, 70%, 80%, 90%, or 95%, or from about 50% to about 99%, or alternatively from about 40% to about 80%, and wherein said taste receptor cell is a human taste receptor cell.

Any amount of the compound of any of Formulae I-V that provides the desired degree of inhibition can be used. For example, a compound of any of Formulae I-V may be used at a concentration of about 0.1 μM to about 1,000 μM to inhibit a taste receptor cell. Alternatively, concentrations of about 1 μM, 50 μM, or 100 μM of a compound of any of Formulae I-V may be used to inhibit the depolarization of a taste receptor cell.

In certain embodiments, a single dose or two to four divided daily doses, provided on a basis of about 0.001 to 100 mg per kilogram of body weight per day, preferably about 0.01 to about 25 mg/kg of body weight per day is appropriate. When inhibiting a taste receptor cell in vivo, the compound of any of Formulae I-V is preferably administered orally.

In one embodiment, the method comprises contacting a taste receptor cell with a compound of any of Formulae I-V, or a pharmaceutically acceptable salt or stereoisomer thereof, wherein said taste receptor cell can detect a sweet, bitter, sour, salty, or umami taste. In another embodiment, the method comprises administering a compound of any of Formulae I-V, or a pharmaceutically acceptable salt or stereoisomer thereof, to a subject in an amount sufficient to inhibit the depolarization of a taste receptor cell. Furthermore, when administered orally, the compound may be dispersed or diluted by saliva.

In another embodiment, compounds of any of Formulae I-V, and a pharmaceutically acceptable salts and stereoisomers thereof, are useful for inhibiting a taste, such as an undesirable taste of a food product. Examples of food products having an undesirable taste include, but are not necessarily limited to, citrus fruits, such as grapefruit, orange, and lemon; vegetables such as tomato, pimento, celery, melon, carrot, potato and asparagus; seasoning or flavoring materials, such as soy sauce and red pepper; soybean products; fish products; meats and processed meats; dairy products, such as cheese; breads and cakes; and confectioneries, such as candies, chewing gum and chocolate. Other examples of food products envisioned in accordance with the present invention are described below and throughout the specification.

The method may be performed such that the taste of the food product being inhibited by the compound of any of Formulae I-V is inhibited by at least about 10%, 20%, 30%, 40%, 50%, 60%, 70%, 80%, 90%, or 95%, or from about 60% to about 99%, or alternatively from about 20% to about 50%. Thus, in a more specific embodiment, the method comprises administering a food product comprising one or more food ingredients and one or more compounds of any of Formulae I-V, or a pharmaceutically acceptable salt or stereoisomer thereof, wherein the one or more compounds according to any of Formulae I-V are present in an amount sufficient to inhibit a bitter taste, produced by the food product, by at least about 10%, 20%, 30%, 40%, 50%, 60%, 70%, 80%, 90%, or 95%, or from about 60% to about 99%, or alternatively from about 30% to about 70%. Of course, in other embodiments, a taste may be inhibited to differing extents.

Any amount of the compound of any of Formulae I-V that provides the desired degree of taste inhibiting can be used. For example, a compound of any of Formulae I-V may be used at a concentration of about 0.1 μM to about 5,000 μM to inhibit a bitter taste. Alternatively, concentrations of about 1 μM, 100 μM, or 500 μM of a compound of any of Formulae I-V may be used to inhibit a sweet taste.

A food product also includes beverages and drinks. Examples of drinks having an undesirable or unwanted taste include, but are not limited to, juices of citrus fruits and vegetables, soybean, milk, coffee, cocoa, black tea, green tea, fermented tea, semi-fermented tea, refreshing drinks, beverages and milk. In certain embodiments, the taste inhibiting effective amount of a compound of any of Formulae I-V, or a pharmaceutically acceptable salt or stereoisomer thereof, has a range of from about 0.01 to about 5.0 grams per 100 mL. In other embodiments, the taste inhibiting effective amount of a compound of any of Formulae I-V, or a pharmaceutically acceptable salt or stereoisomer thereof, has a range of from about 0.5 to about 2 grams per 100 mL. Alternatively, a compound according to any of Formulae I-V, or a pharmaceutically acceptable salt or stereoisomer thereof, is administered in an amount of about 1 gram per 100 mL.

The method of the present invention in its various embodiments may be used to inhibit one or more tastes selected from the group consisting of sweet, bitter, sour, salty, and umami. Preferably, the method of the present invention inhibits a bitter and/or sweet taste and/or umami.

As used herein, the phrase “inhibit a taste” and grammatical variants thereof, such as “taste inhibiting” and “inhibiting a taste,” refers to interfering with the perception of a taste. The taste may be sensed to a lesser degree or not sensed at all by application of the present invention.

In one embodiment, the present invention is directed to a method of inhibiting a taste of a pharmaceutical composition, comprising administering a compound according to any of Formulae I-V, or a pharmaceutically acceptable salt or stereoisomer thereof, to a subject receiving the pharmaceutical composition. The compound of any of Formulae I-V may be administered together with the pharmaceutical composition as a separate composition, for example either concurrently or sequentially. The compound of any of Formulae I-V may administered, or caused to be administered, prior to the pharmaceutical agent producing the taste to be inhibited. Alternatively, the compound of any of Formulae I-V may be administered as a component of the pharmaceutical composition.

By way of example, the method may be performed such that the taste being inhibited by the compound of any of Formulae I-V is inhibited by at least about 10%, 20%, 30%, 40%, 50%, 60%, 70%, 80%, 90%, or 95%, or from about 60% to about 99%, or alternatively from about 25% to about 50%. Thus, in a more specific embodiment, the method comprises administering a pharmaceutical composition comprising a pharmaceutically active agent, optionally one or more excipients, and one or more compounds of any of Formulae I-V, wherein the one or more compounds of any of Formulae I-V are present in an amount sufficient to inhibit a bitter taste, produced by the pharmaceutically active agent, by at least about 10%, 20%, 30%, 40%, 50%, 60%, 70%, 80%, 90%, or 95%, or from about 60% to about 99%, or alternatively from about 30% to about 60%. In another embodiment, the compound of any of Formulae I-V is administered in a ratio of from about 10:1 to about 1:10 in relation to the pharmaceutical agent.

By way of additional examples, the method of inhibiting a taste of a pharmaceutical composition may comprise inhibiting a taste produced by one or more agents selected from the group consisting of antipyretics, analgesics, laxatives, appetite depressants, antacidics, antiasthmatics, antidiuretics, agents active against flatulence, antimigraine agents, psychopharmacological agents, spasmolytics, sedatives, antihyperkinetics, tranquilizers, antihistaminics, decongestants, beta-receptor blockers, agents for alcohol withdrawal, antitussives, fluorine supplements, local antibiotics, corticosteroid supplements, agents against goiter formation, antiepileptics, agents against dehydration, antiseptics, NSAIDs, gastrointestinal active agents, alkaloids, supplements for trace elements, ion-exchange resins, cholesterol-depressant agents, lipid-lowering agents, antiarrhythmics, and expectorants. Further specific examples of pharmaceutical compostions in accordance with the method of the invention are described below.

Additionally, the method of inhibiting a taste of a pharmaceutical composition may comprise inhibiting a taste produced by a counterterrorism pharmaceutical. Because of the increased risk of terrorist attacks, such as chemical, nuclear, or biological attacks, the use of counterterrorism pharmaceutical agents is expected to increase in the future. A counterterrorism pharmaceutical agent includes those pharmaceutical agents that are useful in counteracting agents that can be used in a terrorist attack. Agents that have been used in terrorist acts, or considered as useful for carrying out future terrorist acts, include ricin, sarin, radioactive agents and materials, and anthrax. Pharmaceutical agents that counteract these agents are useful as a counterterrorism pharmaceutical. Such counterterrorism pharmaceuticals include, but are not limited to, antiobiotics such as ciprofloxacin and doxycycline; potassium iodide; and antiviral agents. Thus, in one embodiment of the present invention, the method may be performed such that the taste of a counterterrorism pharmaceutical, such as an antiobiotic such as ciprofloxacin and doxycycline; potassium iodide; or an antiviral agent, is inhibited by the compound of any of Formulae I-V by at least about 10%, 20%, 30%, 40%, 50%, 60%, 70%, 80%, 90%, or 95%, or from about 60% to about 99%, or alternatively from about 25% to about 50%. In another embodiment, the compound of any of Formulae I-V is administered in a ratio of from about 10:1 to about 1:10 in relation to the counterterrorism agent.

In another embodiment, compounds of any of Formulae I-V, or a pharmaceutically acceptable salt or stereoisomer thereof, are useful for inhibiting an undesirable taste of a nutriceutical composition. Examples of nutriceutical compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine. Further specific examples of nutraceutical compostions in accordance with the method of the invention are described below.

By way of example, the method of inhibiting an undesired taste of a nutriceutical composition may be performed such that the taste being inhibited by the compound of any of Formulae I-V is inhibited by at least about 10%, 20%, 30%, 40%, 50%, 60%, 70%, 80%, 90%, or 95%, or from about 60% to about 99%, or alternatively from about 20% to about 50% Thus, in a more specific embodiment, the method comprises administering a nutraceutical composition, comprising a nutraceutical agent, optionally one or more excipients, and one or more compounds of any of Formulae I-V, or a pharmaceutically acceptable salt or stereoisomer thereof, wherein the one or more compounds according to any of Formulae I-V are present in an amount sufficient to inhibit an undesired taste, produced by the nutraceutical agent, by at least about 10%, 20%, 30%, 40%, 50%, 60%, 70%, 80%, 90%, or 95%, or from about 60% to about 99%, or alternatively from about 10% to about 50%.

A compound according to any of Formulae I-V, or a pharmaceutically acceptable salt or stereoisomer thereof, may be incorporated into medical and/or dental compositions. Certain compositions used in diagnostic procedures have an unpleasant taste, such as contrast materials and local oral anesthetics. The compounds of the invention may be used to improve the comfort of subjects undergoing such procedures by improving the taste of compositions. In addition, the compounds of the invention may be incorporated into pharmaceutical compositions, including tablets and liquids, to improve their flavor and improve patient compliance particularly where the patient is a child or a non-human animal.

In another embodiment, compounds of any of Formulae I-V, and pharmaceutically acceptable salts and stereoisomers thereof, can be used to inhibit a taste of a cosmetic product. For example, but not by way of limitation, a compound according to any of Formulae I-V may be incorporated into face creams, lipsticks, lipgloss, and the like. Also, a compound according to any of Formulae I-V, or a pharmaceutically acceptable salt or stereoisomer thereof, can be used to inhibit an unpleasant taste of a lipbalm, such as Chapstick® or Burt's Beeswax® Lip Balm.

In addition, compounds of any of Formulae I-V, and a pharmaceutically acceptable salts and stereoisomers thereof, can be incorporated into compositions that are not traditional foods, pharmaceuticals, or cosmetics, but which may contact taste membranes. Examples include, but are not limited to, soaps, shampoos, toothpaste, denture adhesive, and glue on the surfaces of stamps and envelopes. Thus, the present invention is also directed to a process of preparing a composition that is not a traditional food, pharmaceutical, or cosmetic, but which may contact taste membranes, according to conventional methods, wherein the improvement comprises adding a compound of any of Formulae I-V, or a pharmaceutically acceptable salt or stereoisomer thereof, to said composition.

In another embodiment, a compound according to any of Formulae I-V, or a pharmaceutically acceptable salt or stereoisomer thereof, is used to inhibit a bitter taste associated with one or more the following: bitter nitrogen-containing pharmaceuticals, such as acetaminophen, ampicillin, chlorpheniramine, chlarithromycin, doxylamine, guaifenesin, ibuprofen, pseudoephidrine hydrochloride, and ranitidine, bitter pharmaceutical metallic salts, such as zinc containing bioadhesives (denture adhesive), bitter vitamins, bitter components of foods, such as creatine, limonin, naringin, quinizolate, and bitter components of beverages, such as caffeine, and humulone. In one embodiment, the concentration of the compound according to any of Formulae I-V used is in the range of 0.01 mM to 20 mM and may vary depending on the amount of bitter compound used and its bitterness.

In one embodiment, the present invention is directed to a method of inhibiting the taste of a veterinary product, such as veterinary medicines, veterinary food products, veterinary supplements, and the like, that are administered to domesticated animals. In a preferred embodiment, a compound according to any of Formulae I-V, or a pharmaceutically acceptable salt or stereoisomer thereof, is used to inhibit a taste of a veterinary product administered to a cat or a dog.

In one embodiment, in each of the methods of inhibiting a taste described herein, a compound of any of Formulae I-V, or a pharmaceutically acceptable salt or stereoisomer thereof, is administered in an amount effective to inhibit said taste. As a nonlimiting example, the taste inhibiting effective amount of a compound according to any of Formulae I-V, or a pharmaceutically acceptable salt or stereoisomer thereof, administered in one embodiment is from about 0.01 to about 5.0 grams per 100 mL.

In other embodiments, in the taste inhibiting methods described herein, a compound according to any of Formulae I-V, or a pharmaceutically acceptable salt or stereoisomer thereof, is administered in an amount that is sufficient, in combination with the administration of one or more additional taste inhibiting agents, to inhibit said taste. For example, in a method of inhibiting the bitter taste of a liquid pharmaceutical composition, the composition comprises a compound according to any of Formulae I-V, or a pharmaceutically acceptable salt or stereoisomer thereof, and another taste inhibiting agent, wherein the amount of the compound of any of Formulae I-V is about 25% to about 75% of the amount required to inhibit the bitter taste in the absence of the other taste inhibiting agent.

In another embodiment, the present invention is directed to a method of decreasing the palatability and/or intake of food, comprising administering to a subject in need of such treatment one or more compounds of any of Formulae I-V, or a pharmaceutically acceptable salt or stereoisomer thereof, in an amount sufficient to decrease the palatability and/or intake of food.

Taste modulating protein knockout mice have been shown to have diminished taste preference for sucrose, artificial sweeteners, and umami flavors and diminished taste aversion to bitter solutions. See Zhang et al., Cell 112:293-301 (2003). Thus, according to the present invention, a compound of any of Formulae I-V, or a pharmaceutically acceptable salt or stereoisomer thereof, may be administered to a subject so that the palatability of food, as experienced by said subject, is decreased. Without being bound by theory, it is believed that a lower palatability of food can lead to a lower intake of food by the subject. Thus, in certain embodiments, by administering a compound according to any of Formulae I-V, or a pharmaceutically acceptable salt or stereoisomer thereof, to a subject, the subject will consume a decreased amount of food compared to the subject's food intake when not being administered the compound of the present invention. In other embodiments, by administering a compound according to any of Formulae I-V, or a pharmaceutically acceptable salt or stereoisomer thereof, to a subject, the subject will have a lower caloric intake compared to the subject's caloric intake when not being administered the compound. In other embodiments, administering a compound of any of Formulae I-V, or a pharmaceutically acceptable salt or stereoisomer thereof, to a subject can be a dieting means to facilitate or aid weight loss.

In each of the embodiments of methods described above, the subject of the method, unless otherwise limited to, can be any animal which is in need of the particular treatment or effect of the method. Such animals include, but are not limited to a cow, horse, sheep, pig, chicken, turkey, quail, cat, dog, mouse, rat, rabbit, monkey, or guinea pig. In other embodiments, the animal is a livestock animal, a domesticated animal, or an animal kept as a pet. In particular embodiments, the subject of the claimed method is a human.

Furthermore, in each of the embodiments of the methods described herein, a compound of any of Formulae I-V may be used in varying ratios to the agent that is believed to cause the unwanted taste, such as a bitter or sweet taste. For example, a compound of any of Formulae I-V may be administered in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unwanted taste. In another example, the present invention is directed to a method of inhibiting a bitter taste of a pharmaceutical composition, comprising administering to a subject in need of such method a pharmaceutical composition and a compound of any of Formulae I-V, wherein the pharmaceutical composition comprises a pharmaceutically active agent and optionally one or more excipients, and wherein the compound according to any of Formulae I-V is administered as either a component of the pharmaceutical composition or as a separate dosage form, and wherein the molar ratio of the compound of any of Formulae I-V to the pharmaceutically active agent is about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of any of Formulae I-V can be used, with modifications if preferred, in each of the embodiments described herein.

In one embodiment, the present invention is directed to a method for enhancing insulin secretion, comprising administering an effective amount of a compound of any of Formulae I-V, or a pharmaceutically acceptable salt or stereoisomer thereof. The compound can be administered to a cell or to a whole organism in order to obtain the enhanced insulin secretion. Additionally, the compound of the present invention can be administered by itself or together with an agent known to cause the release of insulin secretion, such as glucose. By way of example, the compound of the present invention used in this method exhibits an IC₅₀ of 1 μM or less, preferably of 100 nM or less, in the assay for membrane potential and calcium mobolization. In another embodiment, the compound of the present invention used in this method inhibits the TRPM5 receptor by at least 75%, preferably 90%, at a concentration of 5 μM or less.

The term “diabetes” refers to one metabolic disorder in which there is impaired glucose utilization inducing hyperglycemia. An overview of the pathogenesis and morphology of diabetes and its late complications is available to practitioners of the art, for instance, in Robins' Pathologic Basis of Disease (5^{th} Ed. pp. 910-922).

The term “insulin resistance syndrome” (IRS) refers to the cluster of manifestations which include insulin resistance; hyperinsulinemia; non insulin dependent diabetes mellitus (NIDDM); arterial hypertension; central (visceral) obesity; and dyslipidemia. In addition to the major late-stage complications of NIDDM (diabetic angiopathy, atherosclerosis, diabetic nephropathy, diabetic neuropathy, and diabetic ocular complications such as retinopathy, cataract formation and glaucoma), many other conditions are linked to NIDDM, including dyslipidemia glucocorticoid induced insulin resistance, dyslipidemia, polycysitic ovarian syndrome, obesity, hyperglycemia, hyperlipidemia, hypercholerteremia, hypertriglyceridemia, hyperinsulinemia, and hypertension. Brief definitions of these conditions are available in any medical dictionary, for instance, Stedman's Medical Dictionary.

As mentioned above, compounds of the present invention can be used to stimulate insulin release. Such activity may be in vitro or in vivo. The amount of the compound of any of Formulae I-V, or a pharmaceutically acceptable salt or stereoisomer thereof, used to stimulate insulin release may not necessarily be the same when used in vivo compared to in vitro. Factors such as pharmacokinetics and pharmacodynamics of the particular compound may require that a larger or smaller amount of the compound of any of Formulae I-V, or a pharmaceutically acceptable salt or stereoisomer thereof, be used when increasing insulin release in vivo.

In one embodiment, the present invention is directed to a method of treating diabetes mellitus in an animal, preferably a mammal, in need thereof, comprising administering to the subject an effective amount of a compound of any of Formulae I-V, or a pharmaceutically acceptable salt or stereoisomer thereof. In another embodiment, the present invention is directed to a method of preventing diabetes mellitus in an animal, preferably a human or other mammal, in need thereof, comprising administering to a subject an insulin secretion enhancing amount of a compound of any of Formulae I-V, or a pharmaceutically acceptable salt or stereoisomer thereof.

The present invention is directed to a method for treating insulin resistance syndrome in an animal, preferably a human or other mammal, in need thereof, comprising administering the animal an effective amount of a compound of any of Formulae I-V, or a pharmaceutically acceptable salt or stereoisomer thereof. The present invention also directed to a method for treating or preventing insulin resistance in a mammal, comprising administering to said mammal an effective amount of a compound of any of Formulae I-V, or a pharmaceutically acceptable salt or stereoisomer thereof.

Further, the present invention is directed to a method of treating hyperglycemia in an animal, preferably a human or other mammal, in need thereof, comprising administering to the animal an effective amount of a compound of any of Formulae I-V, or a pharmaceutically acceptable salt or stereoisomer thereof. The method also includes a method of preventing hyperglycemia in an animal, preferably in a human or other mammal, comprising administering to the animal an insulin secretion enhancing amount of a compound of any of Formulae I-V, or a pharmaceutically acceptable salt or stereoisomer thereof.

The present invention is also directed to a method of enhancing GLP-1 release from a cell, comprising administering an effective amount of a compound of any of Formulae I-V, or a pharmaceutically acceptable salt or stereoisomer thereof.

Thus, in one aspect of this embodiment, the present invention is directed to a method of stimulating release of GLP-1 by administering of a compound of any of Formulae I-V, or a pharmaceutically acceptable salt or stereoisomer thereof, to a subject in need thereof. In certain embodiments, the present invention provides a method of enhancing the release of GLP-1 from intestinal cells, such as small and large intestine cells, with the subsequent effect of enhancing the release of insulin from pancreatic cells.

The compound of any of Formulae I-V, or a pharmaceutically acceptable salt or stereoisomer thereof, can be administered to a cell or to a whole organism to obtain the enhanced GLP-1 release. Additionally, the compound of the present invention can be administered by itself or together with an agent known to cause the release of GLP-1 secretion, such as glucose. By way of example, the compound of the present invention exhibit an IC₅₀ of about 1 μM or less, preferably of about 100 nM or less, in the assay for membrane potential and calcium mobolization. In some embodiments, the compound of the present invention used in the method inhibits the TRPM5 receptor by at least 75%, preferably 90%, at a concentration of 5 μM or less.

As used herein, “GLP-1” or glucagon-like peptide-1 (GLP-1), is a gastrointestinal protein hormone which enhances insulin secretion by administration of nutrient, such as glucose, carbohydrate, fat, proteins, or mixed amino acids. The physiological roles of GLP-1 and the various proposed mechanisms of GLP-1 release after nutrient ingestion are described, for instance, by Kreymann et al., Lancet 2:1300-1304 (1987) and by Deacon, Regulatory Peptides 128: 117-124 (2005). Unless otherwise stated, “GLP-1” means GLP-1 (7-37). By custom in the art, the amino-terminus of GLP-1 (7-37) has been assigned number 7 and the carboxy-terminus, number 37. The amino acid sequence of GLP-1 (7-37) is well-known in the art, but is presented as follows: NH₂-His-Als-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-COOH.

In certain embodiments, the compounds of the present invention cause an increase of GLP-1 release of about 5% to about 50%, or from about 10% to about 70%, or from about 25% to about 100%. In other embodiments, the compounds of the present invention increase the amount of GLP-1 released from the intestinal cells by about 25%, 50%, 75%, or 100%.

Preferably, the method of increasing GLP-1 release is used on a mammal, such as a human or other primate. In other instances, the subject of the method can be a pet, such as a dog or cat.

Detection of GLP-1 release can be performed based on methods known to one of ordinary skill in the art, including those described herein. See, e.g., F. Reinmann, et al., Diabetes, 55(Supp. 2): S78-S-85 (2006).

In one embodiment, the present invention is directed to a method of decreasing gastric secretion and emptying in an animal, preferably a human or other mammal, in need thereof comprising administering to the animal an effective amount of a compound of any of Formulae I-V, or a pharmaceutically acceptable salt or stereoisomer thereof.

Further, in one embodiment the present invention is directed to a method of inhibiting food intake in an animal, preferably a human or other mammal, in need thereof, comprising administering to the animal an effective amount of a compound of any of Formulae I-V, or a pharmaceutically acceptable salt or stereoisomer thereof. GLP-1 is known to play a significant role in the regulation of the physiological response to feeding. GLP-1 is processed from proglucagon and is released into the blood from the endocrine L-cells mainly located in the distal small intestine and colon in response to ingestion of a meal. GLP-1 acts through a G protein-coupled cell surface receptor (GLP-1R) and enhances nutrient-induced insulin synthesis and release. GLP-1 stimulates insulin secretion (insulinotropic action) and cAMP formation. GLP-1(7-36) amide stimulates insulin releaser lowers glucagon secretion, and inhibits gastric secretion and emptying. These gastrointestinal effects of GLP-1 are not found in vagotomized subjects, pointing to a centrally-mediated effect. GLP-1 binds with high affinity to isolated rat adipocytes, activating cAMP production (Valverde et al., 1993) and stimulating lipogenesis or lipolysis. GLP-1 stimulates glycogen synthesis, glucose oxidation, and lactate formation in rat skeletal muscle. Thus, based on the inventors observations, the compounds of the present invention can be used to inhibit food intake because the compounds increase GLP-1 release.

In one embodiment, the present invention is directed to a method of decreasing glucagon secretion in an animal, preferably a human or other mammal, in need thereof comprising administering to the animal an effective amount of a compound of any of Formulae I-V, or a pharmaceutically acceptable salt or stereoisomer thereof.

Glucagon is a hormone consisting of a straight-chain polypeptide of 29 amino acid residues, extracted from pancreatic alpha cells. Its physiological roles, such as elevating blood glucose concentration and activating hepatic phosphorylase, are available to practioners of the art, for instance, in Stedman's Medical Dictionary, 26^{th} Ed. (1990) at 729.

In one embodiment, the present invention is directed to a method of enhancing insulin sensitivity in an animal, preferably a human or other mammal, in need thereof comprising administering to the animal an effective amount of a compound of any of Formulae I-V, or a pharmaceutically acceptable salt or stereoisomer thereof.

In one embodiment, the present invention is directed to a method of increasing beta cell mass of the islets of Langerhans and insulin gene expression in an animal, preferably a human or other mammal, in need thereof, comprising administering to the animal an effective amount of a compound of any of Formulae I-V, or a pharmaceutically acceptable salt or stereoisomer thereof.

In another embodiment, the present invention is directed to a method of treating or preventing obesity in an animal, preferably a human or other mammal, in need thereof comprising administering to the animal an effective amount of a compound of any of Formulae I-V, or a pharmaceutically acceptable salt or stereoisomer thereof.

As used here, “obesity” refers to an abnormal increase of fat in the subcutaneous connective tissues. Stedman's Medical Dictionary, 26^{th} Ed. (1990) at 1235.

In each of the embodiments of methods described above, the subject of the method, unless otherwise limited to, can be any animal which is in need of the particular treatment or effect of the method. Such animals include, but are not limited to, a cow, horse, sheep, pig, chicken, turkey, quail, cat, dog, mouse, rat, rabbit, monkey, or guinea pig. In other embodiments, the animal is a livestock animal, a domesticated animal, or an animal kept as a pet. In particular embodiments, the subject of the claimed method is a human.

In general, however, a suitable dose will be in the range of from about 0.005 to about 100 mg/kg, e.g., from about 0.1 to about 75 mg/kg of body weight per day, such as 0.03 to about 50 mg per kilogram body weight of the recipient per day, preferably in the range of 0.06 to 90 mg/kg/day, most preferably in the range of 0.15 to 60 mg/kg/day. In other embodiments, the suitable dosage will be about 0.1 mg/day to about 2000 mg/day, administered as a single dosage or multiple doses throughout the day.

The compound may conveniently be administered in unit dosage form; for example, containing 0.05 to 1000 mg, conveniently 0.1 to 750 mg, most conveniently, 0.5 to 500 mg of active ingredient per unit dosage form.

The compound of any of Formulae I-V, or a pharmaceutically acceptable salt or stereoisomer thereof, can be typically present in a dosage form in an amount ranging from about 0.1% to about 100% by weight, preferably about 1% to about 80% by weight. The present invention also contemplates an amount of about 1% to about 50%, preferably about 5% to about 20%, about 8%, 15%, or 18%, by weight, of the dosage form.

Ideally, the compound of any of Formulae I-V should be administered to achieve peak plasma concentrations of the active compound of from about 0.005 to about 75 μM, preferably, about 0.01 to 50 μM, most preferably, about 0.02 to about 30 μM. This may be achieved, for example, by the intravenous injection of a 0.0005 to 5% solution of the active ingredient, optionally in saline, or orally administered as a bolus containing about 0.01-1 mg of the active ingredient. Desirable blood levels may be maintained by continuous infusion to provide about 0.0001-5 mg/kg/hr or by intermittent infusions containing about 0.004-15 mg/kg of the active ingredient(s).

The method may be performed such that the insulin secretion, GLP-1 secretion, or insulin sensitivity being enhanced by a compound of any of Formulae I-V is enhanced by at least about 10%, 20%, 30%, 40%, 50%, 60%, 70%, 80%, 90%, or 95%, or from about 60% to about 99%, or alternatively from about 20% to about 50%. Thus, in a more specific embodiment, the method comprises administering a dosage form comprising one or more compounds of any of Formulae I-V, wherein the one or more compounds of any of Formulae I-V are present in an amount sufficient to enhance insulin secretion, GLP-1 secretion, or insulin sensitivity by at least about 10%, 20%, 30%, 40%, 50%, 60%, 70%, 80%, 90%, or 95%, or from about 60% to about 99%, or alternatively from about 30% to about 70%. Of course, in other embodiments, the insulin secretion, GLP-1 secretion, or insulin sensitivity may be enhanced to differing extents.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

In another embodiment, the above described compounds may be used to enhance insulin or GLP-1 secretion from a cell or enhance insulin sensitivity of a cell. Such enhancement may be in vitro or in vivo. The amount of a compound of any of Formulae I-V, or a pharmaceutically acceptable salt or stereoisomer thereof, used to enhance insulin secretion, insulin sensitivity, or GLP-1 secretion may not necessarily be the same when used in vivo compared to in vitro. Factors such as pharmacokinetics and pharmacodynamics of the particular compound may require that a larger or smaller amount of the compound of any of Formulae I-V, or a pharmaceutically acceptable salt or stereoisomer thereof, be used when pancreatic cell in vivo. Accordingly, one aspect of the present invention is a method of enhancing insulin release and GLP-1 secretion from a cell or enhancing insulin sensitivity of a cell, comprising contacting the cell with a compound of any of Formulae I-V, or a pharmaceutically acceptable salt or stereoisomer thereof.

In one aspect of this embodiment, the method comprises contacting a cell, preferably a pancreatic cell, with a compound of any of Formulae I-V, or a pharmaceutically acceptable salt or stereoisomer thereof, wherein said cell secretes insulin.

In one embodiment of this aspect of the present invention, the method comprises contacting a cell, preferably an L-type enteroendocrine cell, with a compound of any of Formulae I-V, or a pharmaceutically acceptable salt or stereoisomer thereof, wherein said cell secretes GLP-1.

In one embodiment, the present invention is also directed to a method of enhancing insulin and GLP-1 release from a cell or enhancing insulin sensitivity of a cell, comprising contacting said cell with a compound of any of Formulae I-V, or a pharmaceutically acceptable salt or stereoisomer thereof, and enhancing the insulin secretion, GLP-1 secretion, or insulin sensitivity of the cell by at least about 10%, 20%, 30%, 40%, 50%, 60%, 70%, 80%, 90%, or 95%, or from about 50% to about 99%. In one aspect of this embodiment, the method comprises contacting said cell with a compound of any of Formulae I-V, or a pharmaceutically acceptable salt or stereoisomer thereof, and enhancing the insulin secretion, GLP-1 secretion, or insulin sensitivity of the cell by about 10% to about 50%. In one embodiment, said cell is a naturally occurring cell. In another embodiment, said cell is a naturally occurring human insulin secreting cell or GLP-1 secreting cell.

Any amount of the compound of any of Formulae I-V that provides the desired degree of enhancement can be used. For example, a single dose or two to four divided daily doses, provided on a basis of about 0.001 to 100 mg per kilogram of body weight per day, preferably about 0.01 to about 25 mg/kg of body weight per day is appropriate. The substance is preferably administered orally, but parenteral routes such as the subcutaneous, intramuscular, intravenous or intraperitoneal routes or any other suitable delivery system, such as intranasal or transdermal routes can also be employed.

As used herein, the term “enhancing” and grammatical variants thereof refers to increasing with the amount of or the degree of. For example, enhancing insulin release or GLP-1 release from a cell means increasing the amount of insulin or GLP-1 that is released by the cell. Similarly, enhancing insulin sensitivity of a cell means increasing the degree of insulin sensitivity of a cell. Enhancing includes but is not necessarily limited to modulating, modifying, activating, and the like.

### Compositions

The present invention is also directed to various, useful compositions comprising a compound of any of Formulae I-V or a pharmaceutically acceptable salt or stereoisomer thereof.

In one aspect, the present invention is directed to a pharmaceutical composition comprising a compound of any of Formulae I-V, as defined above, a pharmaceutically acceptable salt or stereoisomer thereof, and one or more pharmaceutically acceptable carriers. Preferred compositions of the present invention are pharmaceutical compositions, comprising a compound selected from one or more embodiments listed above, and one or more pharmaceutically acceptable excipients. Pharmaceutical compositions that comprise one or more compounds of any of Formulae I-V, or a pharmaceutically acceptable salt or stereoisomer thereof, may be used to formulate pharmaceutical drugs containing one or more active agents that exert a biological effect other than taste inhibition and/or inhibition of a taste modulating protein.

For taste inhibiting purposes, the pharmaceutical composition preferably further comprises one or more active agents that exert a biological effect. Such active agents includes pharmaceutical and biological agents that have an activity other than taste inhibition. Such active agents are well known in the art. See, e.g., The Physician's Desk Reference. Such compositions can be prepared according to procedures known in the art, for example, as described in Remington's Pharmaceutical Sciences, Mack Publishing Co., Easton, Pa., USA. In one embodiment, such an active agent includes bronchodilators, anorexiants, antihistamines, nutritional supplements, laxatives, analgesics, anesthetics, antacids, H₂-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. The pharmaceutical composition according to the present invention may comprise one or more compounds according to any of Formulae I-V, as described above, or a pharmaceutically acceptable salt or stereoisomer thereof; an active agent that has a bitter taste; and optionally one or more pharmaceutically acceptable carriers.

In another embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephidrine hydrochloride, pseudoephidrine sulfate, phenylpropanolamine bitartrate, and ephedrine; beta-receptor blockers, e.g., propanolol; agents for alcohol withdrawal, e.g., disulfiram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenyloine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Active substances which have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine.

In another embodiment, the pharmaceutical composition comprises one or more compounds of any of Formulae I-V, or a pharmaceutically acceptable salt or stereoisomer thereof, and at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In another embodiment, the pharmaceutical composition comprises one or more compounds of any of Formulae I-V, or a pharmaceutically acceptable salt or stereoisomer thereof; a biologically active agent that exhibits an activity other than taste inhibition; and at least one amino acid, such as one selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

The pharmaceutical compositions of the present invention can be in any form suitable to achieve their intended purpose. Preferably, however, the composition is one which can be administered buccally or orally. Alternatively, the pharmaceutical composition may be an oral or nasal spray.

The pharmaceutical compositions of the invention can be in any form suitable for administration to any animal that can experience the beneficial effects of one or more compounds according to any of Formulae I-V, or a pharmaceutically acceptable salt or stereoisomer thereof. Foremost among such animals are humans, although the invention is not intended to be so limited. Other suitable animals include canines, felines, dogs, cats, livestock, horses, cattle, sheep, and the like. A veterinary composition, as used herein, refers to a pharmaceutical composition that suitable for non-human animals. Such veterinary compositions are known in the art.

The pharmaceutical preparations of the present invention can be manufactured using known methods, for example, by means of conventional mixing, granulating, dragée-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Pharmaceutical excipients are well known in the art. Suitable excipients include fillers such as saccharides, for example, lactose or sucrose, mannitol or sorbitol, cellulose preparations and/or calcium phosphates, for example, tricalcium phosphate or calcium hydrogen phosphate, as well as binders, such as, starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries are, above all, flow-regulating agents and lubricants, for example, silica, talc, stearic acid or salts thereof, such as, magnesium stearate or calcium stearate, and/or polyethylene glycol. Dragée cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetylcellulose phthalate or hydroxypropylmethyl-cellulose phthalate, are used. Dye stuffs or pigments can be added to the tablets or dragee coatings, for example, for identification or in order to characterize combinations of active compound doses.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In a further embodiment, the invention is directed to a chewable tablet comprising one or more compounds of any of Formulae I-V and one or more biologically active agents. Chewable tablets are known in the art. See, e.g., U.S. Pat. Nos. 4,684,534 and 6,060,078, each of which is incorporated by reference in its entirety.

For taste inhibition purposes, any kind of medicament may be contained in the chewable tablet, preferably a medicament of bitter taste, natural plant extracts or other organic compounds. More preferably, vitamins such as vitamin A, vitamin B, vitamin B₁, vitamin B₂, vitamin B₆, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite may be contained in the core.

In another embodiment, the present invention is directed to an orally disintegrating composition wherein said orally disintegrating composition further comprises one or more compounds of Formula I, or a pharmaceutically acceptable salt or stereoisomer thereof. Orally disintegrating tablets are known in the art. See, e.g., U.S. Pat. Nos. 6,368,625 and 6,316,029, each of which is hereby incorporated by reference in its entirety.

In another embodiment, the present invention is further directed to a nasal composition further comprising one or more compounds of any of Formulae I-V, or a pharmaceutically acceptable salt or stereoisomer thereof. Nasal sprays are known in the art. See, e.g., U.S. Pat. No. 6,187,332. Addition of one or more compounds of any of Formulae I-V, or a pharmaceutically acceptable salt or stereoisomer thereof, to a nasal spray can reduce the experience of an unpleasant taste associated with the composition of the nasal spray. By way of a nonlimiting example, a nasal spray composition according to the present invention comprises water (such as 95-98 weight percent), a citrate (such as 0.02 M citrate anion to 0.06 M citrate anion), a compound according to any of Formulae I-V, and optionally phosphate (such as 0.03 M phosphate to 0.09 M phosphate).

In another embodiment, the present invention is directed to a solid dosage form comprising a water and/or saliva activated effervescent granule, such as one having a controllable rate of effervescence, and a compound according to any of Formulae I-V, or a pharmaceutically acceptable salt or stereoisomer thereof. Effervescent pharmaceutical compositions are known in the art. See, e.g., U.S. Pat. No. 6,649,186, which is incorporated by reference in its entirety.

For taste inhibition purposes, the effervescent composition may further comprise a pharmaceutically active compound. The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition comprising a compound of any of Formulae I-V can further include one or more additional adjuvants and/or active ingredients which can be chosen from those known in the art including flavors, diluents, colors, binders, filler, surfactant, disintegrant, stabilizer, compaction vehicles, and non-effervescent disintegrants.

In another embodiment, the present invention is directed to a film-shaped or wafer-shaped pharmaceutical composition that comprises a compound of any of Formulae I-V, or a pharmaceutically acceptable salt or stereoisomer thereof, and is capable of disintegrating. Such a film-shaped or wafer-shaped pharmaceutical composition can be configured, for example, as quickly disintegrating administration forms, e.g., administration forms disintegrating within a period of 1 second up to 3 minutes, or as slowly disintegrating administration forms, e.g., administration forms disintegrating within a period of 3 to 15 minutes.

The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable are polymers for use in the film-shaped or wafer-shaped pharmaceutical composition include cellulose derivatives, polyvinyl alcohol (e.g. MOWIOL™), polyacrylates, polyvinyl pyrrolidone, cellulose ethers, such as ethyl cellulose, as well as polyvinyl alcohol, polyurethane, polymethacrylates, polymethyl methacrylates and derivatives and copolymerisates of the aforementioned polymers.

In certain embodiments, the total thickness of the film-shaped or wafer-shaped pharmaceutical composition according to the invention is preferably 5 μm up to 10 mm, preferably 30 μm to 2 mm, and with particular preference 0.1 mm to 1 mm. The pharmaceutical preparations may round, oval, elliptic, triangular, quadrangular or polygonal shape, but they may also have any rounded shape.

In another embodiment, the present invention is directed to a composition comprising a medicament or agent contained in a coating that surrounds a gum base formulation and further comprising a taste-inhibiting amount of a compound of any of Formulae I-V, or a pharmaceutically acceptable salt or stereoisomer thereof. Preferably, the coating comprises at least 50% by weight of the entire product. As the center is chewed, the medicament or agent is released into the saliva. For example, U.S. Pat. No. 6,773,716, which is incorporated herein by reference in its entirety, discloses a suitable medicament or agent contained in a coating that surrounds a gum base formulation. One or more compounds of any of Formulae I-V, or a pharmaceutically acceptable salt or stereoisomer thereof, can be used in preparing the coating. The compound of any of Formulae I-V, or a pharmaceutically acceptable salt or stereoisomer thereof, may be present in varying amounts, such as about 30% 50%, 75%, or 90%. In another embodiment, the compound according to any of Formulae I-V may be present in about 30% to about 99%. In other embodiments, the compound of any of Formulae I-V is present in about 1% to about 30%. For taste inhibition purposes, optionally, the composition may further comprise high-intensity sweeteners and appropriate flavors. It has been found that with respect to certain medicaments or agents that may have an astringent or bitter taste that by adding a inhibiting agent to the formulation, that a much more palatable formulation, including the medicament, can be provided. In this regard, even though the medicament in, for example, its powder form may be bitter or have an offensive taste, the matrix used as the coating of the present invention, including the inhibiting agent, will afford a product having acceptable medicinal properties.

In yet another embodiment, the present invention is directed to a process of preparing an improved composition comprising a medicament or agent contained in a coating that surrounds a gum base formulation, wherein the improvement comprises adding a compound of any of Formulae I-V, or a pharmaceutically acceptable salt or stereoisomer thereof, to the coating that surrounds the gum base formulation. The compound of any of Formulae I-V may be added in varying amounts, such as about 30% 50%, 75%, 80%, or 90%, or from about 10% to about 90%. In other embodiments, the compound of any of Formulae I-V is present in about 1% to about 30%.

In a further embodiment, the invention is directed to a pharmaceutical composition suitable for aerosol administration, comprising a compound of any of Formulae I-V, or a pharmaceutically acceptable salt or stereoisomer thereof, and a suitable carrier. The aerosol composition may further comprise a pharmaceutically active agent. Aerosol compositions are known in the art. See, e.g., U.S. Pat. No. 5,011,678, which is hereby incorporated by reference in its entirety. As a nonlimiting example, an aerosol composition according to the present invention may comprise a medically effective amount of a pharmaceutically active substance, one or more compounds of any of Formulae I-V, or a pharmaceutically acceptable salt or stereoisomer thereof, and a biocompatible propellant, such as a (hydro/fluoro)carbon propellant.

In certain embodiments, the pharmaceutical compositions of the invention comprise from about 0.001 mg to about 1000 mg of a compound of any of Formulae I-V, or a pharmaceutically acceptable salt or stereoisomer thereof. In another embodiment, the compositions of the invention comprise from about 0.01 mg to about 10 mg of a compound of any of Formulae I-V, or a pharmaceutically acceptable salt or stereoisomer thereof.

In another embodiment, the composition of the invention comprises a compound of any of Formulae I-V, or a pharmaceutically acceptable salt or stereoisomer thereof, in an amount sufficient to inhibit a taste modulating protein. By way of example, the present invention is pharmaceutical or veterinary composition, comprising a compound of any of Formulae I-V, or a pharmaceutically acceptable salt or stereoisomer thereof, in an amount sufficient to a taste modulating protein by at least about 10%, 20%, 30%, 40%, 50%, 60%, 70%, 80%, 90%, or 95%, or from about 50% to about 99%, or alternatively from about 10% to about 40%.

In another embodiment, the present invention is directed to a nutriceutical composition comprising one or more nutriceuticals, one or more compounds according to any of Formulae I-V, or a pharmaceutically acceptable salt or stereoisomer thereof, and optionally one or more carriers. Additionally, the invention is directed to a process of preparing an improved nutriceutical composition, wherein the improvement comprises adding one or more compounds of any of Formulae I-V, or a pharmaceutically acceptable salt or stereoisomer thereof, to a nutriceutical composition. In certain embodiments, the one or more compounds of any of Formulae I-V, or a pharmaceutically acceptable salt or stereoisomer thereof, are added to a nutriceutical composition in an amount of about 1% to about 50%, or about 5%, 10%, or 15%, by weight.

In another embodiment, the present invention is directed to a dental hygienic composition comprising one or more compounds of any of Formulae I-V, or a pharmaceutically acceptable salt or stereoisomer thereof. Dental hygienic compositions are known in the art and include but are not necessarily limited to toothpaste, mouthwash, plaque rinse, dental floss, dental pain relievers (such as Anbesol™), and the like. For example, the invention includes a dental bleaching composition which comprises one or more compounds of any of Formulae I-V, or a pharmaceutically acceptable salt or stereoisomer thereof, in an amount sufficient to inhibit a bitter taste. Dental bleaching compositions are known in the art. See, e.g., U.S. Pat. No. 6,485,709, which is herein incorporated by reference in its entirety. A dental bleaching composition of the present invention intended for use with dental trays may utilize a sticky carrier formed from a fluid and a thickener. The sticky carrier accordingly may comprise finely divided silica, such as silica fume, dispersed in a liquid, such as a polyol. Examples of suitable polyols include propylene glycol, glycerin, polypropylene glycols, sorbitol, polyethylene glycols and the like. While the carrier preferably includes thickeners, the carrier may also be only a liquid such as water or any of the liquid polyols without any thickeners.

Additionally, the invention is directed to a process of preparing an improved dental hygienic composition, wherein the improvement comprises adding one or more compounds of any of Formulae I-V, or a pharmaceutically acceptable salt or stereoisomer thereof, to a dental bleaching composition. In certain embodiments, the one or more compounds according to any of Formulae I-V are added to a dental hygienic composition in an amount of about 1% to about 20%, preferably about 1% to about 5%, or about 5%, 10%, or 15%, by weight.

In another embodiment, the present invention is directed to a cosmetic product comprising one or more compounds of any of Formulae I-V, or a pharmaceutically acceptable salt or stereoisomer thereof. For example, but not by way of limitation, the cosmetic product comprising a compound of any of Formulae I-V, or a pharmaceutically acceptable salt or stereoisomer thereof, may be a face cream, lipstick, lipgloss, and the like. Other suitable compositions of the invention include lipbalm, such as Chapstick® or Burt's Beeswax® Lip Balm, further comprising one or more compounds of any of Formulae I-V, or a pharmaceutically acceptable salt or stereoisomer thereof.

Additionally, the invention is directed to a process of preparing an improved cosmetic product, wherein the improvement comprises adding one or more compounds of any of Formulae I-V, or a pharmaceutically acceptable salt or stereoisomer thereof, to a cosmetic product. In certain embodiments, the one or more compounds of any of Formulae I-V, or a pharmaceutically acceptable salt or stereoisomer thereof, are added to a cosmetic product in an amount of about 1% to about 20%, preferably about 1% to about 5%, or about 1%, 2%, or 3%, by weight.

In another embodiment, the present invention is directed to a food product, comprising one or more compounds of any of Formulae I-V, or a pharmaceutically acceptable salt or stereoisomer thereof. Preferably, the food product is one which exhibits an undesirable taste, such as a bitter taste, which can be inhibited by a compound of any of Formulae I-V, or a pharmaceutically acceptable salt or stereoisomer thereof. Furthermore, in a preferred embodiment, the food product comprises a compound of any of Formulae I-V, or a pharmaceutically acceptable salt or stereoisomer thereof, in an amount sufficient to inhibit an unpleasant taste.

Specific food products and food ingredients to which one of more compounds of any of Formulae I-V, or a pharmaceutically acceptable salt or stereoisomer thereof, can be added include but are not necessarily limited to, potassium chloride, ammonium chloride, sodium chloride (e.g., table salt), magnesium chloride, halide salts, naringin, caffeine, urea, magnesium sulfate, saccharin, acetosulfames, potassium benzoate, potassium bicarbonate, potassium carbonate, potassium nitrate, potassium nitrite, potassium sulfate, potassium sulfite, potassium glutamate, food preservatives in their physiologically acceptable salts, unsweetened chocolate, cocoa beans, yogurt, preservatives, flavor enhancers, dietary supplements, gelling agents, pH control agents, nutrients, processing aids, bodying agents, dispersing agents, stabilizers, colorings, coloring diluents, anticaking agents, antimicrobial agents, formulation aids, leavening agents, surface active agents, anticaking agents, nutrient supplements, alkali, acids, sequestrants, denuding agents, general purpose buffers, thickeners, cooked out juice retention agents, color fixatives in meat and meat products, color fixatives in poultry and poultry products, dough conditioners, maturing agents, yeast foods, mold retardants, emulsifiers, texturizers, binders, water correctives, miscellaneous and general purpose food additives, tableting aids, lye peeling agents, washing water agents, oxidizers, antioxidants, enzymes, extenders, fungicides, cake mixes, coffee, tea, dry mixes, non-dairy creamers, salts, animal glue adjuvant, cheese, nuts, meat and meat products, poultry and poultry product, pork and pork products, fish and fish products, vegetable and vegetable products, fruit and fruit products, smoked products such as meat, cheese fish, poultry, and vegetables, whipping agents, masticatory substances in chewing gums, dough strengtheners, animal feed, poultry feed, fish feed, pork feed, defoaming agents, juices, liquors, substances or drinks containing alcohol, beverages including but not limited to alcoholic beverages and non-alcoholic carbonated and/or non-carbonated soft drinks, whipped toppings, bulking agents used in eatables including but not limited to starches, corn solids, polysaccharides and other polymeric carbohydrates, icings, as well as potassium-containing or metal-containing substances with undesirable tastes and the like.

Moreover, the present invention contemplates the preparation of eatables such as breads, biscuits, pancakes, cakes, pretzels, snack foods, baked goods etc. prepared using for example potassium bicarbonate or potassium carbonate in place of the sodium salts as leavening agents in conjunction with a compound of any of Formulae I-V, or a pharmaceutically acceptable salt or stereoisomer thereof, in an amount sufficient to eliminate one or more undesirable tastes. The compound of any of Formulae I-V, or a pharmaceutically acceptable salt or stereoisomer thereof, can be typically present in an amount ranging from about 0.001% to about 50% by weight, preferably about 0.1% to about 10% by weight, or alternatively, from 0.1% to about 1% by weight, of the material with the undesirable taste. The present invention also contemplates the preparation of preservatives for eatables comprising the potassium salts of benzoate, nitrate, nitrite, sulfate, and sulfite and so on, in conjunction with an appropriate concentration of a compound of any of Formulae I-V, or a pharmaceutically acceptable salt or stereoisomer thereof, to eliminate undesirable tastes in foodstuffs. Thus, the invention is directed to a process of preparing an improved food product, wherein the improvement comprises adding one or more compounds of any of Formulae I-V, or a pharmaceutically acceptable salt or stereoisomer thereof, to a food product. In certain embodiments, the one or more compounds of any of Formulae I-V, or a pharmaceutically acceptable salt or stereoisomer thereof, are added to a food product in an amount of about 1% to about 20%, preferably about 1% to about 5%, about 1%, 3%, or 4%, by weight.

In another embodiment, the present invention is directed to an animal food product, comprising one or more compounds of any of Formulae I-V, or a pharmaceutically acceptable salt or stereoisomer thereof. The one or more compounds are preferably in an amount sufficient to inhibit one or more undesirable tastes associated with the animal food product. Animal food products are well known in the art, see, e.g., U.S. Pat. No. 6,403,142, and include dog food, cat food, rabbit food, and the like. The animal food product may also be food products useful for feeding livestock, such as cattle, bison, pigs, chicken, and the like. In another embodiment, the animal food composition of the present invention is a solid hypoallergenic pet food comprising a component that contains protein or protein fragments wherein all of said component is partially hydrolyzed and further comprises one or more compounds of any of Formulae I-V, or a pharmaceutically acceptable salt or stereoisomer thereof.

Additionally, the invention is directed to a process of preparing an improved animal food product, wherein the improvement comprises adding one or more compounds of any of Formulae I-V, or a pharmaceutically acceptable salt or stereoisomer thereof, to an animal food product. In certain embodiments, the one or more compounds of any of Formulae I-V, or a pharmaceutically acceptable salt or stereoisomer thereof, are added to an animal food product in an amount of about 1% to about 25%, about 1% to about 10%, or about 5%, 10%, or 15%, by weight.

In further embodiments of the present invention, any of the compositions described herein and containing a compound of any of Formulae I-V may further comprise one or more additional taste masking agents. Such masking agents include but are not limited to the group consisting of sucralose; zinc gluconate; ethyl maltol; glycine; acesulfame-k; aspartame; saccharin; fructose; xylitol; malitol; isomalt; salt; spray dried licorice root; glycyrrhizin; dextrose; sodium gluconate; sucrose; glucono-delta-lactone; ethyl vanillin; and vanillin.

In another embodiment, the present invention is directed to a composition comprising a compound of any of Formulae I-V, or a pharmaceutically acceptable salt or stereoisomer thereof, and a carrier, wherein said carrier is suitable for an assay. Such carriers may include solid carriers and/or liquid carriers. A composition suitable for an assay may, but not necessarily, be sterile. Examples of suitable carriers for assays include dimethylsulfoxide, ethanol, dichloromethane, methanol, and the like. In another embodiment, a composition comprises a compound of any of Formulae I-V, or a pharmaceutically acceptable salt or stereoisomer thereof, and a carrier, wherein the compound is in an amount suitable for inhibiting a taste modulating protein.

In each of the embodiments of the compositions described herein, a compound of any of Formulae I-V, or a pharmaceutically acceptable salt or stereoisomer thereof, may be used in varying ratios to the agent that is believed to cause the unwanted taste, such as a bitter or sweet taste. For example, a composition of the invention may comprise a compound of any of Formulae I-V in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unwanted taste, such as a bitter or sweet taste. In another example, the present invention is directed to a food product comprising one or more food ingredients and a compound of any of Formulae I-V, wherein the molar ratio of the compound of any of Formulae I-V to the food agent that causes, or is believed to cause, a bitter taste about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of any of Formulae I-V can be used, with modifications if preferred, in each of the embodiments described herein.

The present invention is also directed to various compositions useful for treating diabetes mellitus, insulin resistance syndrome, hyperglycemia, and obesity comprising a compound of any of Formulae I-V, or a pharmaceutically acceptable salt or stereoisomer thereof, and a pharmaceutically acceptable carrier. Such as carrier can be selected from pharmaceutically acceptable excipients and auxiliaries. In a preferred embodiment, the compound is present in the composition in an amount that is effective to achieve its intended purpose. In certain embodiments, the pharmaceutical compositions of the invention comprise from about 0.001 mg to about 1000 mg of one or more compounds of any of Formulae I-V. In another embodiment, the compositions of the invention comprise from about 0.01 mg to about 10 mg of one or more compounds of any of Formulae I-V. Useful pharmaceutical compositions include those described above.

A method of the present invention, such as a method of treating diabetes mellitus, insulin resistance syndrome, hyperglycemia, or obesity, can further comprise administering a second therapeutic agent to treat the same condition to the mammal in combination with a compound of any of Formulae I-V. In one embodiment, one or more compounds of any of Formulae I-V can be administered concurrently with the second therapeutic agent. In one embodiment, one or more compounds of any of Formulae I-V can be administered prior to or subsequent to administration of an effective amount of the second therapeutic agent. In one embodiment, the second therapeutic agent is selected from the group consisting of an insulin releaser, a prandial insulin releaser, a biguanide, an insulin sensitizer, insulin, and a dipeptidyl peptidase-4 (DPP4) inhibitor (such as sitagliptin).

The present invention is also directed to various compositions useful for increasing beta cell mass of the islets of Langerhans and insulin gene expression, decreasing gastric secretion and emptying and glucagon secretion, and inhibiting food intake, comprising a compound of any of Formulae I-V, or a pharmaceutically acceptable salt or stereoisomer thereof, and a pharmaceutically acceptable carrier.

### Synthesis of Compounds

The compounds of the present invention can be prepared using methods known to those skilled in the srt in view of this disclosure. For example, compounds of the invention may be prepared by the following methods shown in Schemes 1-5.

Compound of Formula I can be prepared by condensing a hydrazide (101) with a ketone or an aldehyde (102) in a suitable organic solvent, such as ethanol, 2-propanol, tetrahydrofuran, toluene, etc., as shown in Scheme 1. The presence of a dehydrating agent or a water scavenger, such as molecular sieves or dry potassium carbonate, may be added. Acid catalysis may be used to facilitate the condensation. Acid catalysts include, but are not limited to, p-toluenesulfonic acid, methylsulfonic acid, phosphoric acid, and sulfuric acid.

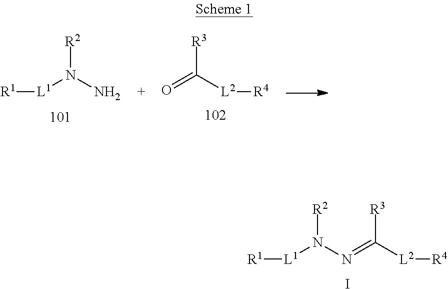

wherein R¹, R², R³, R⁴, L¹, and L² are as defined above for Formula I.

In an alternative process, certain compounds according to Formula I, wherein R² is H and L¹ contains a carbonyl group (C═O) adjacent to the N(R²) group of Formula I can be prepared as shown in Scheme 2. According to this process, a carboxylic acid or acid chloride (103) is treated with a hydrazone (104) to provide a compound according to Formula I. If a carboxylic acid is used, a coupling reagent, such as those commonly used for peptide couplings (carbonyldiimidazole, HBTU, PyBOP, BOP, HATU, EDAC, etc.), is needed. In these cases, a coupling catalyst, such as those commonly used for peptide couplings, such as HOBT, N-hydroxysuccinimide, benzotriazole, etc., may be added. When an acid chloride is used, an acid scavenger, such as triethylamine or an inorganic base, such as potassium carbonate, is added.

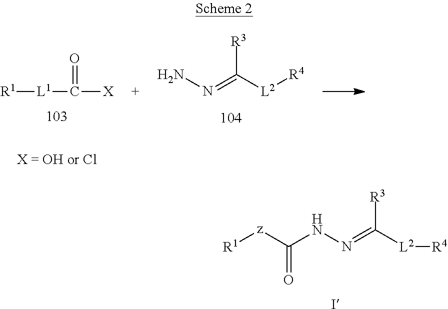

wherein R¹, R³, R⁴, and L² are as defined for Formula I and -z-C(O)-comprises a subset of L¹.

Additional compounds of Formula I, wherein R¹-L¹- is

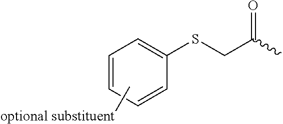

can be prepared from intermediate 107 as shown below in Scheme 3. Intermediate 107 is prepared as shown in the scheme below. A thiophenol (105) is treated with a base, such as potassium carbonate, another carbonate base, or a stronger base, such as sodium hexamethyldisilazide or sodium hydride, and ethyl bromoacetate (or methyl bromoacetate, etc.) to provide intermediate 106. Intermediate 106 is treated with an excess of hydrazine in an alcoholic or other solvent to provide intermediate 107 which is used as describe herein.

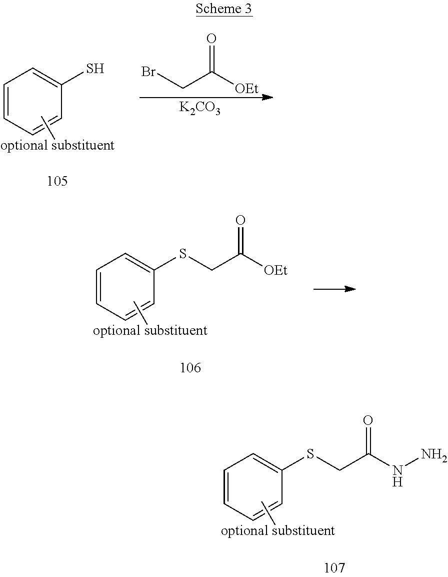

Further, compounds of Formula I, wherein R¹-L¹ is

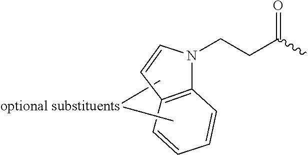

can be prepared from intermediate 110 as shown below in Scheme 4. Intermediate 110 is prepared as shown in the scheme below. An indole (108) is treated with ethyl acrylate or with ethyl 3-bromopropionate and a base to provide 109. Intermediate 109 is treated with an excess of hydrazine in an alcoholic or other solvent to provide 110 which is used as describe herein.

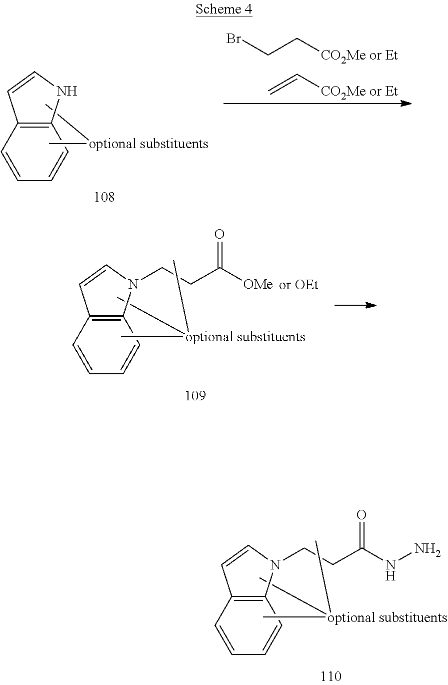

Additional compounds of Formula I, wherein R² is methyl and L¹ contains a carbonyl group linked to N(R²) (such that -zz-C(O)— comprises a subset of L¹) can be prepared as shown in Scheme 5:

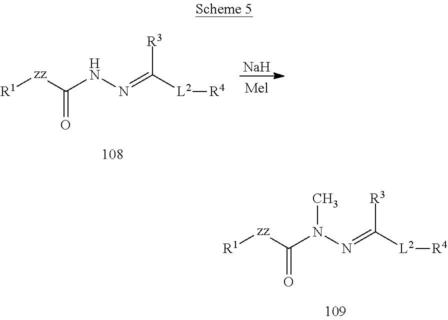

wherein R¹, R³, R⁴, and L² are as defined for Formula I and -zz-C(O)— is a subset of L¹.

Accordingly, a compound of structure 108 is allowed to react with sodium hydride or other base (sodium hexamethyldisilazide or the like) and then with methyl iodide or an alternate methylating agent (dimethyl sulfate, etc.) to produce a compound of structure 109.

Additional functional groups and substituents as defined for Formula I can be easily incorporated into the target molecules using modifications of the methods described. The steps involved to reach the desired target compounds are common organic transformations, such as functional group transformations and protection/deprotection steps, and are known to those skilled in the art of organic synthesis. (For example, see Larock, R. C. Comprehensive Organic Transformations: a Guide to Functional Group Preparations, Second Ed., Wiley-VCH, New York, 1999 and Wuts, P. G. M. and Greene, T. W. Greene's Protective Groups in Organic Synthesis, Fourth Ed., J. Wiley and Sons, New York, 2007.)

Of course, other methods and procedures known in the art may be used to prepare certain compounds of Formula I.

### Testing of Compounds

### Assay for Testing TRPM5 Inhibiting Activity

The activity of compounds of the present invention on human TRPM5 ion channel was measured using the FLIPR-based (fluorescent imaging plate reader) assay for membrane potential and calcium mobilization described in U.S. Patent Application Publication No. 2008/0249189 A1 in live cells, wherein the assay is performed at 30° C. Determinations of affinity and efficacy were derived from measurements of TRPM5 activity over a range of compound concentrations. The concentration range was achieved by 10 serial dilutions (either 2-fold or 3-fold decrements) from a maximum concentration of no greater that 100 μM. Curver fitting was performed on the resulting data to yield concentration-effect functions, from which the half-maximal inhibitory concentration (IC₅₀) was derived for all the tested compounds. The IC₅₀ values were considered to be accurate reflections of affinity between the compounds and TRPM5.

### Testing for Taste Inhibiting Activity

The activity of any of compounds of Formula I for inhibiting a taste can be determined by testing said compound using a number of methods known in the art. For example, one can evaluate the ability of a compound to inhibit a bitter taste by using an in vivo taste assay. This in vivo assay identifies the bitter blockers that by testing their activity using human subjects. A concentration of the bitter compound quinine in water is found that the subject rates as 5 for bitterness on a scale of 0 to 10, where 0 is no bitterness and 10 is the most intense bitterness the subject has ever encountered. This concentration of quinine is then made up containing a concentration of a compound of Formula I to be tested, and the subject rates the bitterness of this solution on the same scale.

### Assay for Determining Insulin Release Enhancement by TRPM5 Inhibitors

Assay I: Insulin release enhancement by TRPM5 inhibitors was tested by the following procedure. Beta-TC-6 cells are an insulin-secreting cell line derived from transgenic mice expressing the large T-antigen of simian virus 40 (SV40) in pancreatic beta-cells as described by Poitout et al., Diabetes, 44:306-313 (1995). The cell line was obtained from ATCC cell bank CAT# CRL-11506 and grown in Dulbecco's Modified Eagle's Medium (DMEM) with 15% fetal bovine serum (FBS), 4 mM glutamine, 4.5 g/L glucose, 110 mg/L sodium pyruvate and 1× penn/strep antibiotic mix in a 37° C. incubator with 5% CO₂. Cultures were routinely split 1:2 twice a week with the aid of trypsin.

The day before the assay 0.15×10⁶ Beta-TC-6 cells were plated into each well of 96 well plates (Greiner bio-one V-bottom, Cat#: 651 180). The cells were spun at 1200 rpm for 5 min and cultured overnight. The next day, growth media was removed from the plate and the plates were washed twice with Krebs-Ringer Bicarbonate Buffer (KRBB) consisting of 118.4 mmol/liter NaCl, 4.69 mmol/liter KCl, 1.2 mmol/liter MgSO₄, 1.18 mmol/liter KH₂PO₄, 2.4 mmol/liter CaCl₂, and 20 mmol/liter NaHCO₃ (equilibrated with 95% O₂ and 5% CO₂, pH 7.4) supplemented with BSA to a final concentration of 0.1% BSA. After each wash, the plates were incubated for 30 min at 37° C. and 5% CO2. The buffer was removed using Platemate 2×3 and replaced with 100 μL of the compounds in 3 mM Glucose in KRBB buffer with 0.1% BSA. The final DMSO concentration in the assay buffer was 0.1%. After 2 hours of static incubation at 37° C. and 5% CO2, 60 μL of the supernatant was collected using Platemate 2×3 and frozen for insulin release measurements. Insulin release in the supernatant was assessed using a commercially available ELISA kit (Mercodia Cat#: 10-1149-10) according to the manufacturer's protocol. The data were expressed as pg of insulin per number of cells.

Alternative Assay 2: Alternatively, insulin release enhancement by TRPM5 inhibitors was be tested by the following procedure. The cell line was obtained from ATCC cell bank CAT# CRL-11506 and grown in Dulbecco's Modified Eagle's Medium (DMEM) with 15% fetal bovine serum (FBS), 4 mM glutamine, 4.5 mM glucose 1500 mg/L sodium bicarbonate and 1× penn/strep antibiotic mix in a 37° C. incubator with 5% CO₂. Cultures were routinely split 1:2 twice a week with the aid of trypsin.

On the day before the assay 0.1×10⁶ Beta-TC-6 cells were plated into each well of 96 well plates, and the cells cultured overnight in growth media.

On the next day, growth media was removed from the plate and the monolayers rinsed with phosphate buffered saline (PBS) and pre-incubated 30 mins at 37° C. in Krebs-Ringer Bicarbonate Buffer (KRBB) consisting of 118.5 mM NaCl, 2.54 mM CaCl₂, 1.19 mM KH₂PO₄, 4.74 mM KCl, 25 mM NaHCO3, 1.19 mM MgSO4, 10 mM HEPES buffer and 0.1% bovine serum albumin at pH 7.4. This buffer was removed and replaced with 100 μL of the same buffer containing various insulin release modulators: including various concentrations of glucose up to 12 mM and compounds up to 100 μM. Compounds stock solutions, e.g. 10-20 mM, were diluted in DMSO. Final DMSO concentrations in incubation buffer were 0.5% or less. Vehicle controls were included. Static incubations were then performed for 2 h at 37° C. The entire incubation volume was collected after the 2 h incubation for insulin assay.

ELISA Protocol for insulin determinations. The following protocol was used for doing ELISA using the Rat/Mouse Insulin ELISA Kit from Linco Research, Inc., CAT# EZRMI-13K and Amplex® Red Hydrogen Peroxide/Peroxidase Assay Kit, CAT# A22188, and Amplex® Red reagent (10-acetyl-3,7-dihydroxyphenoxazine) CAT# A12222.

All reagents were Pre-warmed to room temperature before setting up experiment.

1. Dilute the 10× washing buffer into 1× washing buffer. 50 mL+450 mL de-ionized water. Do 1:10 dilution to the cell culture supernatant (Beta-TC-6 cell incubation).

2. Use one column (8 wells) wells for standard samples and QC1 and QC2, typically 0 and 5 ng/mL of insulin. On some plates full standard curves for insulin obtained as shown below.

3. Cover the unused wells well. Wash each well 3 times with 300 μL of 1× washing buffer each time. (2 minutes each washing step on the shaker.) Decant Wash Buffer and remove the residual amount from all wells by inverting the plate and tapping it smartly onto absorbent towels several times. Do not let wells dry before proceeding to the next step.

4. Add 20 μL Assay Buffer into that blank well and 10 μL into those wells for standards and samples.

5. Add 10 μL rat insulin standards and samples to the appropriate wells.

6. Add 80 μL Detection Antibody to all wells. Cover the plate and incubate at room temperature for 2 hours.

7. Tear off the plate cover and decant solutions from the plate. Tap to remove residual solutions in well.

8. Wash well 3 times with diluted wash buffer 300 μL per well per wash (2 minutes each washing step on the shaker). Tap to remove residual solutions as before.

9. Add 100 μL Enzyme Solution to each well. Cover plate with sealer and incubate with moderate shaking at room temperature for 30 min on the microtiter plate shaker.

10. Remove sealer, decant solutions from the plate and tap plate to remove the residual fluid.

11. Wash wells 6 times with diluted Wash Buffer, 300 μL per well per wash. Decant and tap after each wash to remove residual buffer.

12. Prepare 5 mL working solution of 100 μL Amolex Red reagent containing 2.0 mM H₂O₂. 4.45 mL of 1× Reaction buffer+50 μL 10 mM Amplex Reagent+500 μL 20 mM H₂O₂.

13. Add 100 μL of the Amplex Red reagent/H₂O₂ to each well.

14. Incubate the reactions. Incubate at room temperature for 30 minutes, protected from light (using foil to wrap the plate). Then measure the fluorescence at 590 nm (the excitation range is 530-560 nm) at multiple time points in a Molecular Devices FlexStation.

The above procedure or minor variations thereof were used to determine the insulin-secreting enhancement of the TRPM5 inhibitors.

### Assay for Determining GLP-1 Release by TRPM5 Inhibitor Using GLUTag Cells

Mouse GLUTag cells are a native intestinal cell line which expresses TRPM5. The cell line was obtained from Dr. Daniel J. Drucker in the Division of Endocrinology, Department of Medicine, at University of Toronto, and grown in Invitrogen Dulbecco's Modified Eagle's Medium (DMEM) high glucose (Cat#: 11995) with 10% fetal bovine serum (FBS) and 1× penn/strep antibiotic mix in a 37° C. incubator with 5% CO₂.

Assay 1: All reagents and media were pre-warmed to room temperature prior to the beginning of the experiment.

Seeding of Cell Plate: BD 96-well Matrigel-coated plates (Fisher, Cat #: 08-774-166) were rehydrated using 100 μL of plating media (Invitrogen Cat #: 31985) OPTI-Modified Eagle's Medium (MEM) with 10% FBS and 1× penn/strep antibiotic mix and incubated for 30 minutes in a 37° C. incubator and 5% CO₂. The rehydration media was aspirated and GLUTag cells were plated at 100 ul per well at a density of 7.5×10⁵ cells/ml. The plates were then incubated overnight in a 37° C. incubator and 5% CO₂.

The following protocol utilized the Millipore GLP-1 ELISA Kit (Cat. #: EGLP-35K) to analyze GLP-1 secretion from GLUTag cells in the presence of TRPM5 inhibitors/enhancers and glucose.

ELISA Assay Day 1: Stock solutions and dilution plates of TRPM5 inhibitors or enhancers were prepared. The experiments were carried out in Krebs-Ringer bicarbonate buffer (118.4 mmol/liter NaCl, 4.69 mmol/liter KCl, 1.2 mmol/liter MgSO₄, 1.18 mmol/liter KH₂PO₄, 2.4 mmol/liter CaCl₂, and 20 mmol/liter NaHCO₃ (equilibrated with 95% O₂ and 5% CO₂, pH 7.4) supplemented to a final concentration of 0.1% BSA). The plates were washed twice with the aforementioned KRBB. After each wash, the plates were incubated for 30 min at 37° C. and 5% CO2. The buffer was removed and replaced with 100 μL of the compounds in 0.3 mM Glucose in KRBB supplemented to a final concentration of 0.1% BSA. The final DMSO concentration in the above buffer was 1%. After 2 hours of static incubation at 37° C. and 5% CO2, 50 uL of the supernatant was transferred, using Platemate 2×3, to a 96 well GLP-1 assay plate from Millipore GLP-1 ELISA Kit (Cat. #: EGLP-35K). During the last 30 min of the 2 hour incubation, the GLP-1 plates were prepared as follows. The GLP-1 plates coated with anti-GLP-1 monoclonal antibody were washed three times with 300 μL/well of Wash Buffer (1:10 dilution of Wash Buffer concentrate (10 mM PBS buffer containing Tween 20 and sodium azide)). Assay Buffer in the amount of 100 μL was added to the GLP-1 standard wells. A combination of Assay Buffer (148 μL) and dipeptidyl peptidase IV (DPP-IV) inhibitor (Linco Cat# DPP4) (2 μL) in a total amount of 150 μL was added to all of the cell sample wells. GLP-1 amide ELISA standards (GLP-1 (7-36 amide) in Assay Buffer: 2, 5, 10, 20, 50, and 100 pM) in the amount of 100 μL were added in ascending order in duplicate to the appropriate wells. Samples in the amount of 50 μL were then added to the remaining wells from cell plates. ELISA Plates were shaken gently for proper mixing. The ELISA plates were then covered with an adhesive seal and incubated overnight (20 to 24 hours) at 4° C.

ELISA Assay Day 2: Liquid from the ELISA plates was then decanted, and excess fluid was tapped out on absorbent towels. ELISA Plates were washed 5 times with 300 μL of Wash Buffer per well with a 5-minute incubation at room temperature during the fourth wash. Excess buffer was tapped out on absorbent towels after the fifth wash. Detection Conjugate (Anti GLP-1 Alkaline Phosphate Conjugate) in the amount of 200 μL was then immediately added in each well, followed by a 2-hour incubation period at room temperature. The Detection Conjugate was then decanted, and each well was then washed 3 times with 300 μL of Wash Buffer. Excess fluid was tapped out on paper towels. Diluted Substrate in 200 μL was added in each well and incubated at least 20 minutes at room temperature in the dark. The light sensitive Substrate MUP (Methyl Umbelliferyl Phosphate) was supplied in 10 mg in the Millipore's GLP-1 ELISA Kit and hydrated in 1 mL deionized water just before use. A 1:200 dilution was made in Substrate Diluent (e.g., 100 μL hydrated substrate in 20 mL substrate diluent). Dilution was made fresh each time just before use. After 20 minutes, plates were read at 355 nm/460 nm. When there was sufficient signal-to-noise ratio within the lowest point on standard curve (i.e. 2 pM) and the highest standard point (i.e., 100 pM) within the maximum relative fluorescence unit (RFU) read-out of plate reader, no additional incubation period was required. Otherwise, additional incubation time was required.

When the signal was sufficient, Stop Solution in the amount of 50 μL was added to each well in the same order that the Substrate was added, followed by a 5-minute incubation period in the dark at room temperature to arrest phosphatase activity. ELISA Plates were then read on a fluorescence plate reader with an excitation/emission wavelength of 355 nm/460 nm.

Alternative Assay 2: All reagents and media were pre-warmed to room temperature prior to the beginning of the experiment. Substrate diluent and the light-sensitive Substrate were thawed out right before use.

Seeding of Cell Plate: BD 96-well Matrigel-coated plates (Fisher, Cat #: 08-774-166) with GLUTag cells were rehydrated using 100 μL of plating media (Invitrogen's (Cat #: 31985) OPTI-Modified Eagle's Medium (MEM) with 10% FBS and 1× penn/strep antibiotic mix) and incubated for 30 minutes in a 37° C. incubator with 5% CO₂. GLUTag cells were then trypsinized and counted. A cell dilution with seeding density of 7.5×10⁵ cells/ml of GLUTag cells was created. The rehydration media was aspirated from the Matrigel coated plate. 100 μL of cell dilution were plated into each well of the plate. The plates were then incubated overnight in a 37° C. incubator with 5% CO₂.

The following protocol utilized the Millipore GLP-1 ELISA Kit (Cat. #: EGLP-35K) to analyze GLP-1 secretion from GLUTag cells in the presence of TRPM5 inhibitors/enhancers and glucose.

ELISA Assay Day 1: Stock solutions and dilution plates of TRPM5 inhibitors or enhancers were prepared. 1% of bovine serum albumin (BSA) was added to Krebb's Ringer Bicarbonate Buffer (KRBB) right before use. KRBB consists of 118.5 mM NaCl, 2.54 mM CaCl₂.2H₂O, 1.19 mM KH₂PO4, 4.74 mM KCl, 25 mM NaHCO₃, 1.19 mM MgSO4.7H₂O, and 10 mM HEPES buffer at pH 7.4. The cell plates were incubated with 100 μL of KRBB for 30 minutes. The KRBB buffer was then aspirated. This incubation step was repeated once. The KRBB buffer was aspirated and replaced with 150 μL of the same buffer containing various concentration of TRPM5 inhibitors or enhancers and glucose, i.e. 12.5 mM glucose and 1.5 μM TRPM5 inhibitors. KRBB buffer without the TRPM5 inhibitors/enhancers and glucose were also tested in triplicates. Static incubations of treated cells were then performed for 2 h in a 37° C. incubator with 5% CO₂.

During the last 30 minutes of the two-hour incubation, 96-well ELISA plates were prepared as follows. GLP-1 (Active) ELISA Plates coated with anti-GLP-1 monoclonal antibody were washed three times with 300 μL/well of Wash Buffer (1:10 dilution of Wash Buffer concentrate (10 mM PBS buffer containing Tween 20 and sodium azide). Assay Buffer in the amount of 200 μL (0.05 M PBS at pH 6.8, containing protease inhibitors, with Tween 20, 0.08% sodium azide and 1% BSA) was then added to the non-specific binding wells A10-A12. Assay Buffer in the amount of 100 μL was added to the GLP-1 standard wells. A combination of Assay Buffer (98 μL) and dipeptidyl peptidase IV (DPP-IV) inhibitor (Linco Cat# DPP4) (2 μL) in a total amount of 100 μL was added to all of the cell sample wells. GLP-1 amide ELISA standards (GLP-1 (7-36 amide) in Assay Buffer: 2, 5, 10, 20, 50, and 100 pM) in the amount of 100 μL were added in ascending order in duplicate to the appropriate wells. Samples in the amount of 100 μL were then added to the remaining wells from cell plates. ELISA Plates were shaken gently for proper mixing.

The ELISA plates were then covered with an adhesive seal and incubated overnight (20 to 24 hours) at 4° C.

Liquid from the ELISA plates was then decanted, and excess fluid was tapped out on absorbent towels. ELISA Plates were washed 5 times with 300 μL of Wash Buffer per well with 5-minute incubation at room temperature in Wash Buffer with the fourth wash. Excess buffer was tapped out on absorbent towels after the fifth wash. Detection Conjugate (Anti GLP-1 Alkaline Phosphate Conjugate) in the amount of 200 μL was then immediately added in each well, followed by a 2-hour incubation period at room temperature. The Detection Conjugate was then decanted, and each well was then washed 3 times with 300 μL of Wash Buffer. Excess fluid was tapped out on paper towels. Diluted Substrate in 200 μL was added in each well and incubated at least 20 minutes at room temperature in the dark. The light sensitive Substrate MUP (Methyl Umbelliferyl Phosphate) was supplied in 10 mg in the Millipore's GLP-1 ELISA Kit and hydrated in 1 mL deionized water just before use. A 1:200 dilution was made in Substrate Diluent (e.g., 100 μL hydrated substrate in 20 mL substrate diluent). Dilution was made fresh each time just before use. After 20 minutes, plates were read at 355 nm/460 nm. When there was sufficient signal-to-noise ratio within the lowest point on standard curve (i.e. 2 pM) and the highest standard point (i.e., 100 pM) within the maximum relative fluorescence unit (RFU) read-out of plate reader, no additional incubation period was required. Otherwise, additional incubation time was required.

When the signal was sufficient, Stop Solution in the amount of 50 μL was added to each well in the same order that the Substrate was added, followed by a 5-minute incubation period in the dark at room temperature to arrest phosphatase activity. ELISA Plates were then read on a fluorescence plate reader with an excitation/emission wavelength of 355 nm/460 nm.

Assay for Determining Insulin Release by TRPM5 Inhibitors from Isolated Primary Rat Islets

Isolation of Rat Islets: Islets were isolated from 226-250 g male Sprague Dawley rats from Taconic. The animals were initially anesthetized with CO2 gas and euthanized by cervical dislocation. The pancreatic duct was injected with 15 to 30 ml of Collagenase P (Roche Diagnostics catalog #1 249 002) at 0.6 mg/ml in Hanks' Balanced Salt Solution (HBSS) (Gibco Catalog #14025-076) supplemented with BSA (Sigma, catalog #A 7409) to a final concentration of 0.1%. The pancreas was excised and placed into a 20 ml scintillation vial, incubated in a water bath at 37° C. for 16 to 18 minutes and then shaken briskly for at least 60 seconds. The digested tissue was poured into a 50 ml conical tube (1 per tube) containing cold Hank's Buffer supplemented with BSA (as above) and washed 7-10 times. During each wash, the tissue was allowed to settle for 4 minutes, supernatant aspirated off, and the tissue re-suspended in the HBSS/0.1% BSA buffer. Big lumps of tissue were removed during the first two washes using a 1 ml pipette. After the last wash, only 5 mls of the digest was left in the conical tube which was then gently layered on a BSA gradient consisting of 10 mls of 35% and 5 mls of 30% BSA and centrifuged for 5 min. at 3400 rpm at 5° C. Islets were harvested from the upper layers with a disposable plastic pipette, placed in a fresh 50 mL conical tube, and filled up with HBSS buffer. After a 1 min spin at 1,000 rpm, the supernatant was removed and the pellet was re-suspended in 20-25 mls of HBSS/0.1% BSA buffer. The islets were picked according to shape, size and color under a microscope using a 200 μl pipette and placed in 3-5 ml of dissociation buffer containing 138 mM NaCl, 5.6 mM KCl, 1.2 mM MgCl2, 1 mM EGTA, 5 mM HEPES, 3 mM D-glucose, 162.5 μg/mL collagenase, 0.1% BSA (pH 7.4) for 5-6 min. The cells were re-suspended in RPMI media (GIBCO Cat#: 11879) with 10% FBS, Pen/Strep and 5 mM Glucose and plated on Greiner—V bottom TC plates (Cat. #: 651180) at 100 μl per well at a density of 2,500-3,000 cells per well. The plates were incubated at 37° C. and 5% CO₂ for 16-20 hours prior to being assayed.

Measurement of Insulin Release: Insulin release measurements were carried out in Krebs-Ringer bicarbonate buffer (118.4 mmol/liter NaCl, 4.69 mmol/liter KCl, 1.2 mmol/liter MgSO₄, 1.18 mmol/liter KH₂PO₄, 2.4 mmol/liter CaCl₂, and 20 mmol/liter NaHCO₃ (equilibrated with 95% O₂ and 5% CO₂, pH 7.4) supplemented with BSA to a final concentration of 0.1%). Islets were washed three times with KRBB/0.1% BSA using Platemate 2×3 (Matrix Thermo-Fisher Scientific) and pre-incubated with 100 μl/well of 3 mM Glucose in KRBB/0.1% BSA for 30 min at 37° C. and 5% CO₂. After an additional wash with KRBB/0.1% BSA, the islets were subjected to testing with 200 μl of compounds of interest in KRBB/0.1% BSA supplemented with 12 mM Glucose for 3 hours at 37° C. and 5% CO₂ to make the final DMSO concentration 0.1%. After incubation, 95 μl of the supernatant was removed using Platemate 2×3 and frozen for insulin release measurements. Insulin release in the supernatant was assessed using a commercially available ELISA kit (Mercodia-Cat. #10-1124-10) according to the manufacturer's protocol. The data were expressed as pg of insulin per number of cells.

The following examples are illustrative, but not limiting, of the compounds, compositions, and methods of the present invention. Suitable modifications and adaptations of the variety of conditions and parameters normally encountered in clinical therapy and which are obvious to those skilled in the art in view of this disclosure are within the spirit and scope of the invention.

### EXAMPLES

The identity of the prepared compounds was confirmed using HPLC and mass spectrometry. Analytical LC-MS was performed using the following LC-MS conditions:

Method A: solvent A: 10 mM ammonium acetate in 95:5 water:acetonitrile, solvent B: 5:95 water:acetonitrile, flow=0.5 mL/min, column=Phenomenex Luna C18 (150 mm×2 mm, 5 μm), 15 min;

Method B: solvent A: 0.1% HCO₂H in water, solvent B: methanol, flow=1.2 mL/min, column=Atlantis C18 (50 mm×4.6 mm, 5 μm), 5 min;

Method C: solvent A: 10 mM NH₄Ac, solvent B: MeCN, flow=1.0 mL/min, column=Atlantis C18 (7.5 mm×4.6 mm, 5 μm), 4.5 min;

Method D: solvent A: methanol with 0.1% TFA, solvent B: water with 0.1% TFA, flow=0.6 mL/min, 85-10% B over first 2 minutes, plateau at 10% for 2.5 minutes, then returning to 85% for 2 minutes, column=Eclipse XDB-C18 (4.6 mm×50 mm, 5 μm); and

Method E: solvent A: 0.1% HCO₂H in water, solvent B: acetonitrile, flow=1.0 mL/min, column=Atlantis C18 (50 mm×4.6 mm, 5 μm), 5 min.

### Example 1

### 2-(3-Chlorophenylthio)-N′-(3,4-dimethoxybenzylidene)propanehydrazide

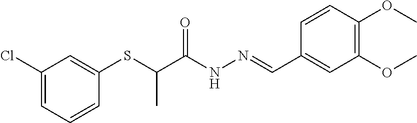

(a) Methyl 2-(3-chlorophenylthio)propanoate: To a stirred solution of 3-chlorobenzenethiol (1.0 g, 6.91 mmol) in CH₃CN (14 mL) was added methyl 2-bromopropionate (0.77 mL, 6.91 mmol) along with K₂CO₃ (1.91 g, 13.83 mmol). The reaction was stirred at room temperature for 60 hours by which time the reaction mixture had become a white slurry. The reaction was diluted with CH₂Cl₂ and washed with water and brine. The organic layer was dried (Na₂SO₄), filtered and concentrated. The product (1.59 g, 99%) was used without further purification.

(b) 2-(3-Chlorophenylthio)propanehydrazide: Methyl 2-(3-chlorophenylthio)propanoate (0.8 g, 3.48 mmol) was dissolved in MeOH (6 mL) and was added dropwise to a stirred solution of hydrazine hydrate (60%, 1.08 mL, 34.8 mmol) in MeOH (4 mL). The reaction mixture was heated to 50° C. via oil bath for 1 hour. The reaction mixture was diluted with CH₂Cl₂ and washed with water (2×50 mL). The organic layer was dried (Na₂SO₄), filtered and concentrated. The product (0.79 g, 98%; white solid) was used without further purification.

(c) 2-(3-Chlorophenylthio)-N′-(3,4-dimethoxybenzylidene)-propane-hydrazide: To a stirred suspension of 2-(3-chlorophenylthio)-propane-hydrazide (533 mg, 2.31 mmol) in absolute EtOH (7.5 mL) was added 3,4-dimethoxybenzaldehyde (462 mg, 2.78 mmol). The reaction mixture was stirred overnight at room temperature and the resulting solid material was collected by filtration. The title compound was obtained as a colorless solid, 806 mg (92%). LCMS m/z 379 (M+H), tR=10.2 min. (Method A).

### Example 2

### N′-(3,4-dimethoxybenzylidene)-3-(1-methyl-1H-indol-3-yl)propanehydrazide

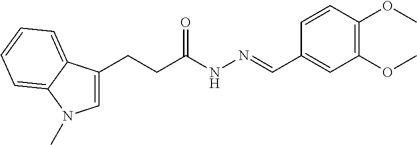

(a) Methyl 3-(1H-indol-3-yl)propanoate: To a stirred solution of 3-indolepropionic acid (5 g, 26.4 mmol) in MeOH (80 mL) was added conc. H₂SO₄ (0.5 mL). The reaction mixture was heated at reflux for 5 hours. The reaction mixture was allowed to cool to room temperature and the reaction solution was reduced to ⅓ volume at reduced pressure. The remaining solution was diluted with water and extracted with CH₂Cl₂. The organic phase was washed with saturated aq. NaHCO₃ and brine and dried (Na₂SO₄). The material was purified on silica gel using EtOAc-hexanes (0 to 30%) and provided 5.16 g (96%) of the desired compound as a pale yellow oil which solidified upon standing.

(b) Methyl 3-(1-methyl-1H-indol-3-yl)propanoate: To a stirred solution of methyl 3-(1H-indol-3-yl)propanoate (350 mg, 1.72 mmol) in DMF (3 mL) at 0° C. was added NaH (85 mg, 2.1 mmol; 60% in mineral oil). The cooling bath was removed after several minutes and the reaction solution was stirred for an additional 5 minutes followed by the addition of CH₃I (0.12 mL, 1.92 mmol). The reaction mixture was stirred for 3 hours at room temperature. The reaction was quenched with aq. NH₄Cl and diluted with EtOAc. The organic phase was washed twice with water and brine and dried (Na₂SO₄). The material was purified on silica gel using EtOAc-hexanes (0 to 20%) and provided 260 mg (70%) of product as a colorless oil.

(c) 3-(1-Methyl-1H-indol-3-yl)propanehydrazide: To a stirred solution of hydrazine hydrate (0.62 mL, 12 mmol; 60%) in MeOH (1 mL) was added a solution of methyl 3-(1-methyl-1H-indol-3-yl)propanoate (260 mg, 1.2 mmol) in MeOH (3 mL total volume). The reaction mixture was heated at 45° C. for 1 hour and then refluxed for 5 hours. The heating was stopped and the reaction mixture was allowed to stir overnight at room temperature, during which time a colorless solid formed. MeOH was removed at reduced pressure and the solid was dissolved in CH₂Cl₂ and a small amount of water was added. The aqueous phase was extracted with CH₂Cl₂ several times and the combined organic layers were dried (Na₂SO₄). The material was purified on silica gel using MeOH—CH₂Cl₂ (0 to 8%) and to give 236 mg (91%) of the product as a colorless solid.

(d) N′-(3,4-Dimethoxybenzylidene)-3-(1-methyl-1H-indol-3-yl)-propanehydrazide: 3-(1-Methyl-1H-indol-3-yl)propanehydrazide (235 mg, 1.08 mmol) and 3,4-dimethoxybenzaldehyde (198 mg, 1.19 mmol) in absolute EtOH (5 mL) were heated at 90° C. for 4 hours. The reaction solution was concentrated and the product was purified on silica gel using MeOH—CH₂Cl₂ (0 to 4%). The solid obtained was dried in vacuo at 50° C. overnight to provide 316 mg of the title compound as a colorless solid. LCMS m/z 366 (M+H), t_{R}=9.6 min (Method A).

### Example 3

### 3-(3-Methyl-1H-indol-1-yl)-N′-((8-methylquinolin-6-yl)methylene)propanehydrazide

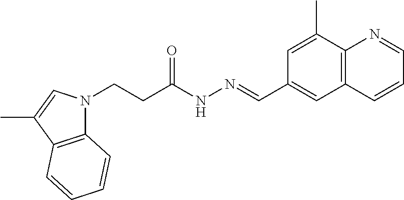

(a) Methyl 3-(3-methyl-1H-indol-1-yl)propanoate: A mixture of 3-methyl-1H-indole (0.5 g, 3.8 mmol), methyl 3-bromopropionate (0.44 mL, 11.0 mmol) and Cs₂CO₃ (3.7 g, 11.0 mmol) in DMF (20 mL) was stirred for 3 days at ambient temperature. The solvent was removed in vacuo and the residue was diluted with EtOAc and washed with water (4×50 mL) and dried (Na₂SO₄). The product was purified on silica gel using EtOAc-hexanes (0 to 10%).

(b) 3-(3-Methyl-1H-indol-1-yl)propanehydrazide: To a solution of hydrazine hydrate (0.53 mL, 11.0 mmol; 60%) in MeOH (10 mL) was added a methanolic solution of methyl 3-(3-methyl-1H-indol-1-yl)propanoate (0.3 g, 1.3 mmol). The reaction mixture was then heated at 70° C. for 4 hours. The solvent was removed under reduced pressure. The residue was diluted with EtOAc and washed with water and brine and dried (Na₂SO₄). Evaporation provided the desired compound as a colorless solid.

(c) 8-Methylquinoline-6-carboxaldehyde: This compound for use in step (d) was prepared according to procedure described in Example 10, steps a)-c), of U.S. Patent Application Publication No. 2008/0214455.

(d) 3-(3-Methyl-1H-indol-1-yl)-N′-((8-methylquinolin-6-yl)-methylene)propanehydrazide: The title compound was prepared from 8-methylquinoline-6-carboxaldehyde and 3-(3-methyl-1H-indol-1-yl)propanehydrazide by methods similar to those described in Examples 1 and 2. LCMS m/z 371 (M+H), t_{R}=4.6 min. (Method B).

### Example 4

### N′-(4-(3-Hydroxypropoxy)-3-methoxybenzylidene)-3-(3-methyl-1H-indol-1-yl)propanehydrazide

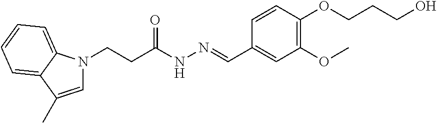

(a) 4-(3-Hydroxypropoxy)-3-methoxybenzaldehyde: To a stirred solution of vanillin (304 mg, 2 mmol) in DMF (4 mL) at room temperature was added 3-iodo-1-propanol (0.29 mL, 3 mmol) and K₂CO₃ (553 mg, 4 mmol). The reaction mixture was stirred at room temperature for 7 hours followed by quenching with aq. NH₄Cl. The product was extracted three times into EtOAc and the combined organic layers were dried (Na₂SO₄). The material was purified on silica gel using EtOAc-hexanes (0 to 70%) and to give 392 mg (93%) of the product as an off-white waxy solid.

(b) N′-(4-(3-Hydroxypropoxy)-3-methoxybenzylidene)-3-(3-methyl-1H-indol-1-yl)propanehydrazide: To a stirred suspension of 3-(3-methyl-1H-indol-1-yl)propanehydrazide (84 mg, 0.39 mmol) in absolute EtOH (4 mL) was added 4-(3-hydroxypropoxy)-3-methoxybenzaldehyde (90 mg, 0.43 mmol). The reaction mixture was warmed with a heat gun until homogeneous and then stirred at room temperature for 24 hours. The reaction volume was reduced to ⅓ volume and hexane was added to precipitate product. The resulting colorless solid was collected by filtration and dried under vacuum, providing 132 mg (83%) of the title compound. LCMS m/z 410 (M+H), t_{R}=9.0 min. (Method A).

### Example 5

### N-(4-Chlorophenethyl)-2-(3,4-dimethoxybenzylidene)hydrazinecarboxamide

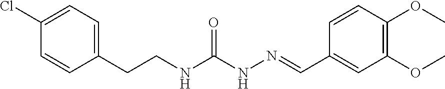

(a) N-(4-Chlorophenethyl)hydrazinecarboxamide: This starting compound was prepared according to Cerecetto et. al., Il Farmaco, 1998, 53, 89-94, using 4-chlorophenethylamine.

(b) N-(4-Chlorophenethyl)-2-(3,4-dimethoxybenzylidene)hydrazine-carboxamide was prepared from N-(4-chlorophenethyl)hydrazinecarboxamide and 3,4-dimethoxybenzaldeyde according to procedures described in Example 1 and 2. LCMS m/z 362 (M+H), t_{R}=4.4 min. (Method B).

### Example 6

### trans-N′-(3,4-Dimethoxybenzylidene)-2-(3-(trifluoromethyl)phenyl)cyclopropanecarbohydrazide

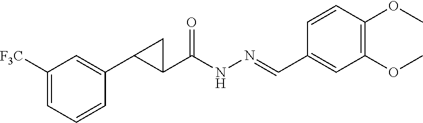

The title compound was prepared according to the method described in Example 31 of U.S. Patent Application Publication No. 2007/0207093 A1, using trans-3-trifluoromethylcinnamic acid instead of 3-chlorocinnamic acid. 3-Trifluoromethylcinnamic acid can be prepared by methods known in the art. LCMS m/z 393 (M+H) t_{R}=4.1 min. (Method C).

### Example 7

### N′-(3,4-Dimethoxybenzylidene)-3-(3,4-dimethyl-1H-indol-1-yl)propanehydrazide

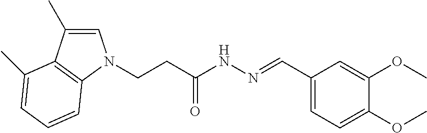

(a) N,N-Dimethyl-1-(4-methyl-1H-indol-3-yl)methanamine: To a stirred solution of dimethylamine (11.1 mL, 22.4 mmol, 2 M in THF) in HOAc (10 mL) at 0° C. was added 37% aq. formaldehyde (1.6 mL, 21.4 mmol) via dropwise addition. The reaction solution was stirred for 45 minutes at 0° C. THF (6 mL) was then added to the reaction solution followed by the dropwise addition of 4-methylindole (2.6 g, 19.82 mmol) in MeOH (10 mL). The reaction mixture was then allowed to warm to room temperature while stiffing overnight. The reaction mixture was diluted with EtOAc and made basic with 2 N NaOH. The organic layer was separated and concentrated down onto silica gel. Purification on silica gel using CH₂Cl₂:MeOH:NH₄OH (0 to 100% 90:10:1) gave the product (3.4 g, 92%) as a tan solid.

(b) 3,4-Dimethyl-1H-indole: A solution of N,N-dimethyl-1-(4-methyl-1H-indol-3-yl)methanamine (3.4 g, 18 mmol) in MeOH was hydrogenated under an atmosphere of hydrogen (balloon) in the presence of 10% Pd/C. After 4.5 hours, LCMS indicated that 65% of the starting material remained. An additional aliquot of Pd/C was added along with a few drops of concentrated HCl and stirring was continued overnight. LCMS indicated that 25% starting material was still remaining. An additional aliquot of Pd/C was added along with a few more drops of concentrated HCl and stirring was continued overnight. The reaction mixture was filtered and the filtrate was diluted with CH₂Cl₂ and made basic with 1 N NaOH. The product was extracted into CH₂Cl₂ and the combined organic layers were concentrated to give 2.6 g of the product as a tan solid which was used directly without additional purification.

(c) Ethyl 3-(3,4-dimethyl-1H-indol-1-yl)propanoate: To a stirred solution of 3,4-dimethyl-1H-indole (2.34 g, 16.1 mmol) in DMF (30 mL) at room temperature was added Cs₂CO₃ (10.5 g, 32.2 mmol) and ethyl acrylate (1.75 mL, 16.1 mmol). The reaction was stirred overnight at room temperature. The reaction was diluted with EtOAc and washed with water and brine and dried (Na₂SO₄). The solution was concentrated down onto silica gel and purified on silica gel using EtOAc-hexanes (0 to 15%). The product was isolated as a colorless viscous oil, 3.44 g (86%).

(d) 3-(3,4-Dimethyl-1H-indol-1-yl)propanehydrazide: To a solution of hydrazine hydrate (6.7 mL, 129 mmol; 60%) in MeOH (50 mL) was added a methanolic solution of ethyl 3-(3,4-dimethyl-1H-indol-1-yl)propanoate (3.44 g, 14.0 mmol). The reaction was then stirred overnight at room temperature. The solvent was removed under reduced pressure and the residue was diluted with CH₂Cl₂ and washed with water. The aqueous layer was extracted several times with CH₂Cl₂ and the combined organic layers were concentrated to dryness. The product was obtained as a colorless solid, 3.2 g (99%).

(e) N′-(3,4-Dimethoxybenzylidene)-3-(3,4-dimethyl-1H-indol-1-yl)propanehydrazide: To a stirred solution of 3-(3,4-dimethyl-1H-indol-1-yl)propanehydrazide (3.23 g, 13.96 mmol) in MeOH at room temperature was added 3,4-dimethoxybenzaldehyde (2.33 g, 13.96 mmol). The reaction became a slurry after 15 minutes and it was stirred overnight at room temperature. The solid was filtered and the title compound was obtained as a colorless solid, 4.5 g (85%). LCMS m/z 380 (M+H), t_{R}=10.5 min. (Method A).

### Example 8

### 3-(3,4-Dimethyl-1H-indol-1-yl)-N′-(2-fluoro-4,5-dimethoxybenzylidene)propanehydrazide

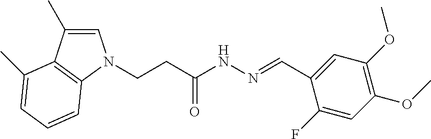

A solution of 3-(3,4-dimethyl-1H-indol-1-yl)propanehydrazide prepared in Example 7d) (45 mg, 0.19 mmol) and 2-fluoro-4,5-dimethoxybenzaldehyde (36 mg, 0.19 mmol) in MeOH was stirred overnight at room temperature. The resulting solid was collected by filtration. The title compound was obtained as a colorless solid, 64 mg (84%). LCMS m/z 398 (M+H), t_{R}=11.0 min. (Method A).

### Example 9

### trans-N′-(3,4-Dimethoxybenzylidene)-N-methyl-2-phenylcyclopropanecarbohydrazide

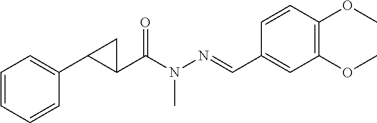

To a stirred solution of N′-(3,4-dimethoxybenzylidene)-2-phenylcyclopropanecarbohydrazide (300 mg, 0.92 mmol) in DMF (3 mL) at 0° C. was added NaH (44 mg, 1.1 mmol, 60% in mineral oil). The cooling bath was removed after 2 minutes and stirring was continued for an additional 5 minutes. To this was added CH₃I (142 mg, 1.0 mmol) in DMF (1 mL total volume). The reaction mixture was stirred at room temperature for 5 hours. The reaction was quenched with aqueous NH₄Cl and the product was extracted into EtOAc. The combined organic layers were washed with brine and dried (Na₂SO₄). Purification on silica gel using EtOAc-hexanes (0 to 30%) provided 297 mg (95%) of the title compound as a colorless solid. LCMS m/z 339 (M+H), t_{R}=11.2 min. (Method A).

### Example 10

### trans-N′-((8-Methylquinolin-6-yl)methylene)-2-phenylcyclopropanecarbohydrazide

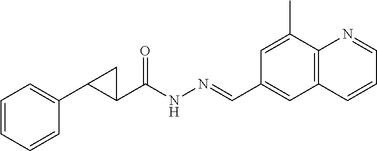

(a) trans-Methyl 2-phenylcyclopropanecarboxylate: To a stirred solution of trans-2-phenylcyclopropanecarboxylic acid (10.3 g, 63.5 mmol) in MeOH at room temperature was added conc. H₂SO₄ (2 mL) and the reaction mixture was refluxed for 4 hours. MeOH was removed in vacuo and the resulting residue was dissolved in EtOAc and extracted with water, brine and dried (Na₂SO₄). Evaporation provided a yellow viscous oil (10 g) which was used without further purification.

(b) trans-2-Phenylcyclopropanecarbohydrazide: To a stirred solution of hydrazine hydrate (18 mL, 341 mmol, 60%) in MeOH (100 mL) at room temperature was added a methanol solution of trans-methyl 2-phenylcyclopropanecarboxylate (10 g, 56.8 mmol). The reaction mixture was stirred at room temperature overnight. The reaction solution was reduced to about ¼-volume and diluted with EtOAc. This was washed with water, brine and dried (Na₂SO₄). Evaporation provided a colorless solid which was used without additional purification.

(c) trans-N′-((8-Methylquinolin-6-yl)methylene)-2-phenylcyclo-propanecarbohydrazide: To a stirred solution of trans-2-phenyl-cyclopropanecarbohydrazide (123 mg, 0.76 mmol) in EtOH (2 mL) at room temperature was added 8-methylquinoline-6-carbaldehyde (130 mg, 0.76 mmol, prepared as described in Example 3(c)). The reaction was stirred overnight providing a colorless solid which was isolated by filtration and dried under vacuum (225 mg, 90%). LCMS m/z 330 (M+H), t_{R}=10.5 min. (Method A).

### Example 11

### trans-N′-((8-Methylquinolin-6-yl)methylene)-2-phenylcyclopropanecarbohydrazide, E1 enantiomer

### trans-N′-((8-Methylquinolin-6-yl)methylene)-2-phenylcyclopropanecarbohydrazide, E2 enantiomer

Racemic trans-N′-((8-methylquinolin-6-yl)methylene)-2-phenyl-cyclopropanecarbohydrazide (appx. 1.2 g) was resolved using chiral Supercritical Fluid Chromatography (SFC) to provide trans-N′-((8-methylquinolin-6-yl)methylene)-2-phenylcyclopropanecarbohydrazide, E1 enantiomer (508 mg, RT=4.45 min) and trans-N′-((8-methylquinolin-6-yl)methylene)-2-phenylcyclopropanecarbohydrazide, E2 enantiomer (509 mg, RT=7.75 min). Preparative method: Chiralcel OD-H (3×15 cm) 08-10148 column; 40% ethanol/CO₂, 100 bar, mobile phase; 85 mL/min flow rate; detector wavelength set at 220 nm; injection volume of 1.5 mL, 17 mg/mL in ethanol/DCM. Analytical method: Chiralcel OD-H (25×0.46 cm) column; 40% ethanol/CO₂, 100 bar, mobile phase; 3 mL/min flow rate; detector wavelength set at 220 nm. E1 enantiomer: Colorless solid, LCMS m/z 330 (M+H), t_{R}=9.8 min. (Method A); E2 enantiomer: Colorless solid, LCMS m/z 330 (M+H), t_{R}=9.8 min. (Method A).

### Example 12

### N′-(4-(Benzyloxy)-3-methoxybenzylidene)-3-(3,4-dimethyl-1H-indol-1-yl)propanehydrazide

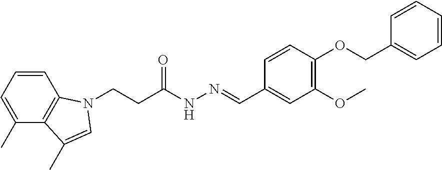

(a) 4-(Benzyloxy)-3-methoxybenzaldehyde: To a stirred solution of vanillin (152 mg, 1 mmol) and ground KOH (168 mg, 3 mmol) in DMF (3.5 mL) at room temperature was added benzyl bromide (0.18 mL, 1.5 mmol). The reaction mixture was stirred overnight at room temperature. The reaction mixture was diluted with EtOAc and water and the aqueous layer was extracted with EtOAc (2×). The combined organic layers were washed with water (2×), brine and dried (Na₂SO₄). Evaporation provided 461 mg of crude product which was used without additional purification.

(b) N′-(4-(Benzyloxy)-3-methoxybenzylidene)-3-(3,4-dimethyl-1H-indol-1-yl)propanehydrazide: 3-(3,4-Dimethyl-1H-indol-1-yl)propane-hydrazide (69 mg, 0.3 mmol) and 4-(benzyloxy)-3-methoxybenzaldehyde (100 mg, 0.3 mmol) were combined in MeOH (2 mL). To this was added 2 drops of acetic acid after which the solution became homogeneous. After stirring at room temperature for 5 min, a colorless precipitate formed. The reaction mixture was stirred for an additional 3 hours. The solid was collected by filtration, washed with MeOH (3 mL) and dried under vacuum to give 139 mg (˜100%) of the title compound as a colorless solid. LCMS m/z 456 (M+H), t_{R}>17 min. (Method A).

### Example 13

### 3-(3,4-Dimethyl-1H-indol-1-yl)-N′-(3-methoxy-4-(pyridin-2-ylmethoxy)benzylidene)propanehydrazide

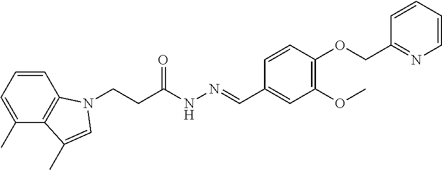

(a) 3-Methoxy-4-(pyridin-2-ylmethoxy)benzaldehyde: Vanillin (152 mg, 1 mmol), 2-picolylchloride hydrochloride (246 mg, 1.5 mmol) and ground KOH (252 mg, 4.5 mmol) were combined in DMF (3.5 mL) and the resulting mixture was stirred at room temperature for 24 hours. The reaction mixture was diluted with EtOAc and water, and the aqueous layer was extracted with EtOAc (2×). The combined organic layers were washed with water (2×), brine and dried (Na₂SO₄). Purification on silica gel using MeOH-DCM (0 to 1.0%) followed by a second purification using EtOAc-DCM (0 to 100%) provided 52.8 mg (22%) of the desired product.

(b) 3-(3,4-Dimethyl-1H-indol-1-yl)-N′-(3-methoxy-4-(pyridin-2-ylmethoxy)benzylidene)propanehydrazide: 3-(3,4-Dimethyl-1H-indol-1-yl)propanehydrazide (51 mg, 0.22 mmol) and 3-methoxy-4-(pyridin-2-ylmethoxy)benzaldehyde (52.8 mg, 0.22 mmol) were combined in MeOH (1.5 mL). To this was added 2 drops of acetic acid after which the solution became homogeneous. The reaction mixture was stirred for 3 hours and the solid was collected by filtration, washed with MeOH (2 mL) and dried under vacuum to give 84 mg (84%) of the title compound as a colorless solid. LCMS m/z 457 (M+H), t_{R}=10.7 min. (Method A).

### Example 14

### N′-(3,4-Dimethoxybenzylidene)-3-(3-methyl-1H-pyrrolo[2,3-b]pyridin-1-yl)propanehydrazide

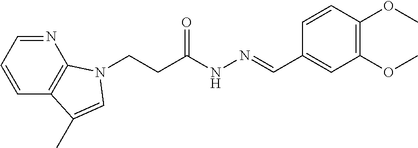

(a) 3-Methyl-1H-pyrrolo[2,3-b]pyridine: This intermediate was prepared using a similar method to that described by Jensen et. al., Angew. Chem. Int. Ed. 2008, 47, 888-890. To a reaction tube was added toluene (5 mL), 2,3-dichloropyridine (300 mg, 2.03 mmol), Pd₂ dba₃ (2 mg, 0.0025 mmol), dppf (6 mg, 0.01 mmol), NaOtBu (487 mg, 5.07 mmol), and allylamine (0.15 mL, 2.03 mmol). The reaction tube was sealed and heated at 140° C. for 20 hours followed by stiffing at room temperature for 24 hours. The reaction mixture was washed with water, brine and dried (Na₂SO₄) and absorbed onto silica gel. Purification on silica gel using DCM-hexane (0 to 60%), followed by EtOAc-hexane (80%) provided a orange solid (100 mg, 37%).

(b) Ethyl 3-(3-methyl-1H-pyrrolo[2,3-b]pyridin-1-yl)propanoate: To a stirred solution of 3-methyl-1H-pyrrolo[2,3-b]pyridine (95 mg, 0.72 mmol), Cs₂CO₃ (468 mg, 1.44 mmol) in DMF (2 mL) was added ethyl acrylate (0.078 mL, 0.72 mmol). The reaction mixture was stirred overnight at room temperature. The reaction was diluted with EtOAc and washed with water, brine and dried (Na₂SO₄). Concentration provided a viscous orange oil (150 mg) which was used without additional purification.

(c) 3-(3-Methyl-1H-pyrrolo[2,3-b]pyridin-1-yl)propanehydrazide: To a stirred solution of hydrazine hydrate (14 mL, 271 mmol, 60%) in MeOH was added the crude ethyl 3-(3-methyl-1H-pyrrolo[2,3-b]pyridin-1-yl)propanoate obtained from 14(b). The reaction mixture was heated at 45° C. for 1 hour. The reaction solution was diluted with EtOAc, washed with sat. aq. NaHCO₃, water and dried (Na₂SO₄). The solvent was evaporated and the residue was triturated with 10% EtOAc-hexane. The resulting tan solid (62 mg) was used directly in the next step without additional purification.

(d) N′-(3,4-Dimethoxybenzylidene)-3-(3-methyl-1H-pyrrolo[2,3-b]pyridin-1-yl)propanehydrazide: To a solution of 3-(3-methyl-1H-pyrrolo[2,3-b]pyridin-1-yl)propanehydrazide (62 mg, 0.28 mmol) in MeOH (1 mL) was added 3,4-dimethoxybenzaldehyde (47 mg, 0.28 mmol). The reaction mixture was stirred at room temperature. 10% EtOAc-hexane was added to the reaction mixture and the tan solid was collected by filtration. The solids were triturated with EtOAc providing the title compound (39.7 mg). LCMS m/z 367 (M+H), t_{R}=8.3 min. (Method A).

### Example 15

### N′-(3-Chloro-4,5-dimethoxybenzylidene)-3-(3,4-dimethyl-1H-indol-1-yl)propanehydrazide

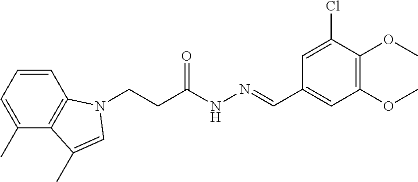

3-(3,4-Dimethyl-1H-indol-1-yl)propanehydrazide prepared in Example 7(d) (50 mg, 0.22 mmol) and 3-chloro-4,5-dimethoxybenzaldehyde (43 mg, 0.22 mmol) were combined in MeOH (2 mL) and stirred overnight at room temperature. The resulting product was isolated by filtration providing the title compound as a colorless solid (75 mg, 84%). LCMS m/z 414 (M+H), t_{R}=12.2 min. (Method A).

### Example 16

### 3-(3,4-Dimethyl-1H-indol-1-yl)-N′-(3-methoxy-4-(pyridin-3-ylmethoxy)benzylidene)propanehydrazide

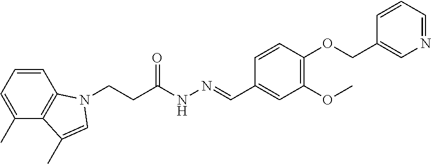

(a) 3-Methoxy-4-(pyridin-3-ylmethoxy)benzaldehyde: This intermediate was prepared according to the procedure outlined in Example 13(a) using 3-(chloromethyl)pyridine hydrochloride (242 mg, 1.48 mmol). The crude product was obtained as a yellow oil (220 mg) and was not subjected to further purification.

(b) 3-(3,4-Dimethyl-1H-indol-1-yl)-N′-(3-methoxy-4-(pyridin-3-ylmethoxy)benzylidene)propanehydrazide: The compound was prepared according to methods already described using 3-(3,4-dimethyl-1H-indol-1-yl)propanehydrazide (52 mg, 0.232 mmol) and 3-methoxy-4-(pyridin-3-ylmethoxy)benzaldehyde (55 mg, 0.23 mmol) in EtOH (2 mL). The title compound was obtained as a colorless solid (95 mg, 92%). LCMS m/z 457 (M+H), t_{R}=10.5 min. (Method A).

### Example 17

### N′-(3,4-Dimethoxybenzylidene)-3-(4-methyl-1H-benzo[d]imidazol-1-yl)propanehydrazide

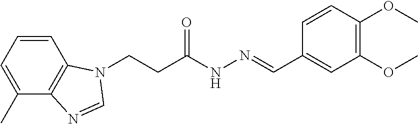

(a) 4-Methyl-1H-benzo[d]imidazole: 3-Methylbenzene-1,2-diamine (400 mg, 3.27 mmol), triethylorthoformate (0.65 mL, 3.92 mmol) and I₂ (83 mg, 0.327 mmol) were combined in MeCN (10 mL) and stirred at room temperature overnight. The reaction mixture was diluted with EtOAc and washed with sat. aq. NaHCO₃ and dried (Na₂SO₄). Purification on silica gel using EtOAc-hexane (0 to 80%) gave the desired product (310 mg, 72%).

(b) Ethyl 3-(4-methyl-1H-benzo[d]imidazol-1-yl)propanoate: The compound was prepared according to methods already described using 4-methyl-1H-benzo[d]imidazole (310 mg, 2.34 mmol), ethyl acrylate (0.26 mL, 2.34 mmol) and Cs₂CO₃ (1.26 g, 3.9 mmol) in DMF (10 mL). The product was obtained as an oil (340 mg, 62%).

(c) 3-(4-Methyl-1H-benzo[d]imidazol-1-yl)propanehydrazide: The compound was prepared according to methods already described using ethyl 3-(4-methyl-1H-benzo[d]imidazol-1-yl)propanoate (340 mg, 1.46 mmol) and hydrazine hydrate (1 mL, 17.6 mmol, 60%) in MeOH. The product was obtained as a yellow solid (200 mg) which was used directly in the next step.

(d) N′-(3,4-Dimethoxybenzylidene)-3-(4-methyl-1H-benzo[d]imidazol-1-yl)propanehydrazide: 3-(4-Methyl-1H-benzo[d]imidazol-1-yl)propane-hydrazide (52 mg, 0.3 mmol) and 3,4-dimethoxybenzaldehyde (50 mg, 0.3 mmol) were combined in MeOH and stirred at room temperature overnight. The resulting yellow homogeneous solution was concentrated and the residue was absorbed onto silica gel. Purification on silica gel using MeOH-DCM (0 to 25%) gave the title compound as an off-white solid (103 mg, 94%). LCMS m/z 367 (M+H), t_{R}=7.5 min. (Method A).

### Example 18

### 3-(3,4-Dimethyl-1H-indol-1-yl)-N′-(3-methoxy-4-(pyridin-4-ylmethoxy)benzylidene)propanehydrazide

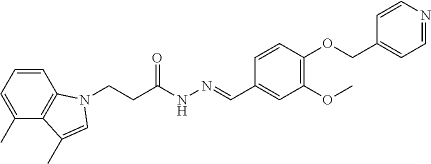

(a) 3-Methoxy-4-(pyridin-4-ylmethoxy)benzaldehyde. This intermediate was prepared according to the procedure outlined in Example 13(a) using 4-picolyl chloride hydrochloride (242 mg, 1.48 mmol). The crude product was obtained as a reddish solid and was used without further purification.

(b) 3-(3,4-Dimethyl-1H-indol-1-yl)-N′-(3-methoxy-4-(pyridin-4-ylmethoxy)benzylidene)propanehydrazide: The compound was prepared by methods already described using 3-methoxy-4-(pyridin-4-ylmethoxy)benzaldehyde (50 mg, 0.2 mmol) and 3-(3,4-dimethyl-1H-indol-1-yl)propanehydrazide (47 mg, 0.2 mmol) in EtOH. The title compound was obtained as a colorless solid (78 mg). LCMS m/z 457 (M+H), t_{R}=10.3 min. (Method A).

### Example 19

### 3-(3,4-Dimethyl-1H-indol-1-yl)-N′-(3-methoxy-4-(pyridazin-3-ylmethoxy)benzylidene)propanehydrazide

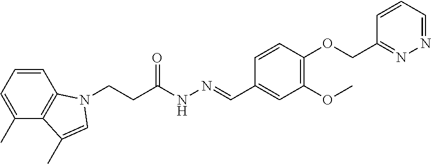

(a) 3-(Chloromethyl)pyridazine: To a stirred solution of 3-methylpyridazine (0.29 mL, 3.19 mmol) in CHCl₃ (6 mL) at 60° C. was added trichloroisocyanuric acid (296 mg, 1.28 mmol) in several portions. The reaction mixture was heated for 2 hours and then absorbed directly onto silica gel. Purification on silica gel using EtOAc-hexane (0 to 80%) provided the product as a reddish oil (160 mg).

(b) 3-Methoxy-4-(pyridazin-3-ylmethoxy)benzaldehyde: This intermediate was prepared according to the procedure outlined in Example 13(a) using 3-(chloromethyl)pyridazine (150 mg, 1.17 mmol). Purification on silica gel using MeOH-DCM (0 to 1.5%) provided the product as a colorless solid (33 mg).

(c) 3-(3,4-Dimethyl-1H-indol-1-yl)-N′-(3-methoxy-4-(pyridazin-3-ylmethoxy)benzylidene)propanehydrazide: The compound was prepared by methods already described using 3-methoxy-4-(pyridazin-3-ylmethoxy)benzaldehyde (33 mg, 0.14 mmol) and 3-(3,4-dimethyl-1H-indol-1-yl)propanehydrazide (31 mg, 0.14 mmol) in MeOH (2 mL). The title compound was obtained as a colorless solid (45 mg, 73%). LCMS m/z 458 (M+H), t_{R}=9.9 min. (Method A).

### Example 20

### trans-N′-(3-Methoxy-4-(pyridin-3-ylmethoxy)benzylidene)-2-phenylcyclopropanecarbohydrazide

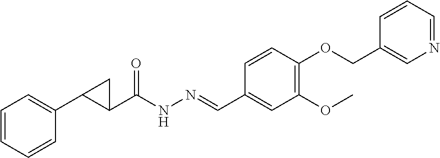

To a stirred solution of trans-2-phenylcyclopropanecarbohydrazide (50 mg, 0.31 mmol) in MeOH (1 mL) at room temperature was added 3-methoxy-4-(pyridin-3-ylmethoxy)benzaldehyde (76 mg, 0.31 mmol). The reaction was stirred for 2 hours providing an orange homogeneous solution. The reaction solution was concentrated to dryness and purified on silica gel using EtOAc-hexane (0-100%). The title compound was obtained as a colorless solid (56 mg, 45%). LCMS m/z 402 (M+H), t_{R}=9.3 min. (Method A).

### Example 21

### 3-(3,4-Dimethyl-1H-indol-1-yl)-N′-(3-methoxy-4-(pyrazin-2-ylmethoxy)benzylidene)propanehydrazide

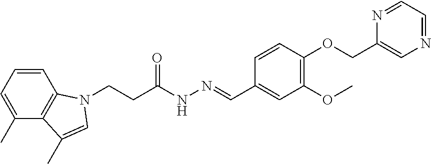

(a) 2-(Chloromethyl)pyrazine: A solution of 2-methylpyrazine (0.75 mL, 8.0 mmol), NCS (1.5 g, 11.4 mmol) and benzoyl peroxide (96 mg, 0.4 mmol) in CCl₄ (30 mL) was refluxed overnight. After cooling to room temperature, the reaction was diluted with EtOAc, washed with water, brine and dried (Na₂SO₄). The crude material obtained (˜600 mg) was used directly in the next step.

(b) 3-Methoxy-4-(pyrazin-2-ylmethoxy)benzaldehyde: To a solution of vanillin (710 mg, 4.66 mmol) and crude 2-(chloromethyl)pyrazine (600 mg, 4.66 mmol) in DMF (10 mL) was added K₂CO₃ (1.93 g, 14 mmol). The reaction was heated at 60° C. for 72 hours. The reaction was diluted with EtOAc, washed with water, brine and dried (Na₂SO₄). Purification on silica gel using MeOH-DCM (0 to 1.5%) provided a light-orange solid (57 mg).

(c) 3-(3,4-Dimethyl-1H-indol-1-yl)-N′-(3-methoxy-4-(pyrazin-2-ylmethoxy)benzylidene)propanehydrazide: The compound was prepared by methods already described using 3-methoxy-4-(pyrazin-2-ylmethoxy)benzaldehyde (50 mg, 0.2 mmol) and 3-(3,4-dimethyl-1H-indol-1-yl)propanehydrazide (47 mg, 0.2 mmol) in MeOH. The title compound was obtained as a colorless solid (72 mg, 77%). LCMS m/z 458 (M+H), t_{R}=10.2 min. (Method A).

### Example 22

### trans-N′-((8-Methoxyquinolin-6-yl)methylene)-2-phenylcyclopropanecarbohydrazide

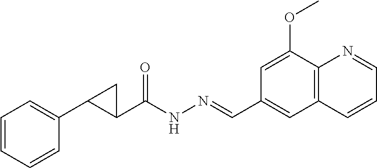

(a) Ethyl 8-methoxyquinoline-6-carboxylate: A mixture of 4-amino-3-methoxybenzoic acid (200 mg, 1.2 mmol), glycerol (0.17 mL, 2.4 mmol), sodium 3-nitrobenzenesulfonate (1.2 g, 5.4 mmol) and 75% H₂SO₄ (4 mL) was heated for 3 hours at 100° C. followed by 1 hour at 140° C. The reaction mixture was allowed to cool to 60° C. and an excess of EtOH was added. The reaction mixture was stirred overnight maintaining a temperature of 60° C. Upon cooling, the reaction solution was poured into a separatory funnel containing aq. NH₄OH and EtOAc (aqueous phase should be basic). The aqueous layer was extracted with EtOAc (2×) and the combined organic layers were washed with brine and dried (Na₂SO₄). Evaporation provided crude product which was used without additional purification.

(b) (8-Methoxyquinolin-6-yl)methanol: To a stirred solution of crude ethyl 8-methoxyquinoline-6-carboxylate (250 mg, 1.08 mmol) in THF (10 mL) at room temperature was added via dropwise addition LiAlH₄ (1.8 mL, 4.32 mmol, 2.4 M in THF). After 20 min, the reaction solution was cooled to 0° C. and quenched by adding water. The mixture was filtered through a pad of Celite and the filtrant was extracted with EtOAc (2×) and the combined organic layers were washed with brine and dried (Na₂SO₄). Purification on silica gel using EtOAc-DCM (0 to 100%) provided the product as a yellow oil (190 mg).

(c) 8-Methoxyquinoline-6-carbaldehyde: A mixture of (8-methoxyquinolin-6-yl)methanol (190 mg, 1.0 mmol) and MnO₂ (217 mg, 2.5 mmol) in EtOH (8 mL) was heated overnight at 70° C. The reaction solution was filtered and the solvent was evaporated, providing the product as a light brown solid (160 mg).

(d) trans-N′-((8-Methoxyquinolin-6-yl)methylene)-2-phenylcyclo-propanecarbohydrazide: To a stirred solution of trans-2-phenylcyclopropanecarbohydrazide (70 mg, 0.4 mmol) in MeOH at room temperature was added 8-methoxyquinoline-6-carbaldehyde (75 mg, 0.4 mmol). The reaction mixture was stirred overnight providing a yellow homogeneous solution. Silica gel was added to the reaction solution and the solvent was evaporated. Purification on silica gel using EtOAc-hexane (0 to 100%) provided the title compound as a colorless solid (96 mg, 70%). LCMS m/z 346 (M+H), t_{R}=9.4 min. (Method A).

### Example 23

### 3-(1-Methyl-1H-indol-3-yl)-N′-((8-methylquinolin-6-yl)methylene)propanehydrazide

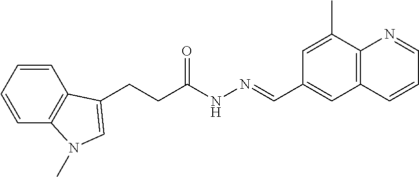

3-(1-Methyl-1H-indol-3-yl)propanehydrazide (170 mg, 0.78 mmol) and 8-methylquinoline-6-carbaldehyde (134 mg, 0.78 mmol) in MeOH (3 mL) were stirred overnight at room temperature. The product was isolated by filtration providing the title compound as a colorless solid (66 mg). LCMS m/z 371 (M+H), t_{R}=10.2 min (Method A).

### Example 24

### trans-2-Phenyl-N′-((8-(trifluoromethyl)quinolin-6-yl)methylene)cyclopropanecarbohydrazide

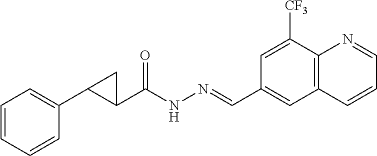

(a) 4-Amino-3-(trifluoromethyl)benzoic acid: A methanol solution of 4-nitro-3-(trifluoromethyl)benzoic acid (1 g, 4.25 mmol) was hydrogenated under an atmosphere of H₂ (balloon pressure) in the presence of 10% Pd/C catalyst at room temperature overnight. The catalyst was filtered off and the solvent was evaporated, providing the product as a colorless solid (870 mg, quantitative yield).

(b) Methyl 8-(trifluoromethyl)quinoline-6-carboxylate: A mixture of 4-amino-3-(trifluoromethyl)benzoic acid (870 mg, 4.2 mmol), glycerol (0.62 mL, 8.5 mmol), sodium 3-nitrobenzenesulfonate (4.3 g, 19.1 mmol) and 75% H₂SO₄ (10 mL) was heated for 3 hours at 100° C. followed by 1 hour at 140° C. The reaction mixture was allowed to cool to 60° C. and an excess of MeOH was added. The reaction was stirred overnight maintaining a temperature of 60° C. Upon cooling, the reaction solution was poured into a separatory funnel containing aq. NH₄OH and EtOAc (aqueous phase should be basic). The aqueous layer was extracted with EtOAc (2×) and the combined organic layers were washed with brine and dried (Na₂SO₄). Purification on silica gel using EtOAc-hexane (0 to 25%) provided the product as a pale yellow solid (530 mg).

(c) (8-(Trifluoromethyl)quinolin-6-yl)methanol: To a stirred solution of methyl 8-(trifluoromethyl)quinoline-6-carboxylate (500 mg, 1.96 mmol) in THF (5 mL) at room temperature was added via dropwise addition LiAlH₄ (0.81 mL, 1.96 mmol, 2.4 M in THF). After 30 min, the reaction solution was quenched by adding water and EtOAc. The aqueous phase was extracted with EtOAc (2×) and the combined organic layers were washed with brine and dried (MgSO₄). The solvent was evaporated providing a yellow viscous oil (400 mg) which was used directly in the next step.

(d) 8-(Trifluoromethyl)quinoline-6-carbaldehyde: A mixture of (8-(trifluoromethyl)quinolin-6-yl)methanol (400 mg, 1.76 mmol) and MnO₂ (460 mg, 5.28 mmol) in acetonitrile was heated overnight at 70° C. The reaction solution was filtered and the solvent was evaporated. Purification on silica gel using EtOAc-hexane (0 to 35%) provided the product as a light yellow solid (245 mg).

(e) trans-2-Phenyl-N′-((8-(trifluoromethyl)quinolin-6-yl)methylene)-cyclopropanecarbohydrazide: To a stirred solution of trans-2-phenylcyclopropanecarbohydrazide (176 mg, 1.0 mmol) in MeOH (5 mL) at room temperature was added 8-(trifluoromethyl)quinoline-6-carbaldehyde (225 mg, 1.0 mmol). The reaction was stirred overnight and the product was isolated by filtration. The title compound was obtained as a colorless solid (302 mg, 79%). LCMS m/z 384 (M+H), t_{R}=9.4 min. (Method A).

### Example 25

### trans-N′-((8-Chloroquinolin-6-yl)methylene)-2-phenylcyclopropanecarbohydrazide

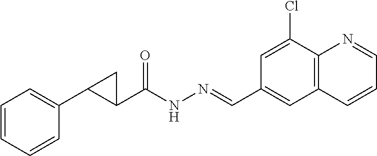

To a stirred solution of trans-2-phenylcyclopropanecarbohydrazide (254 mg, 1.44 mmol) in MeOH at room temperature was added 8-chloroquinoline-6-carbaldehyde (275 mg, 1.44 mmol; prepared according to Example 9 of US 2008/0214455). The reaction mixture was stirred overnight and the product was isolated by filtration. The title compound was obtained as a colorless solid (470 mg, 93%). LCMS m/z 350 (M+H), t_{R}=10.4 min. (Method A).

### Example 26

### 3-(6-Methoxypyridin-2-yl)-N′-((8-methylquinolin-6-yl)methylene)propanehydrazide

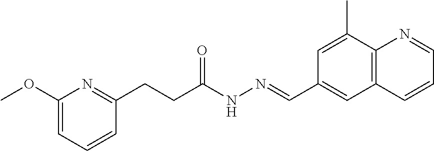

(a) (E)-Methyl 3-(6-methoxypyridin-2-yl)acrylate: To a stirred solution of 6-methoxypyridine-2-carboxaldehyde (0.35 mL, 2.9 mmol) in toluene was added methyl (triphenylphosphoranylidene)acetate (1.95 g, 5.8 mmol) and the reaction was heated at 60° C. overnight. The reaction was diluted with EtOAc and washed with water, brine and dried (Na₂SO₄). Purification on silica gel using EtOAc-hexane (0 to 30%) provided 530 mg (94%) of the desired product.

(b) Methyl 3-(6-methoxypyridin-2-yl)propanoate: (E)-Methyl 3-(6-methoxypyridin-2-yl)acrylate (530 mg, 2.74 mmol) was hydrogenated under an atmosphere of H₂ (balloon pressure) using 10% Pd/C catalyst. When complete, the reaction was filtered through a pad of Celite and the solvent was removed in vacuo.

(c) 3-(6-Methoxypyridin-2-yl)propanehydrazide: To a stirred solution of hydrazine hydrate (0.8 mL, 15.4 mmol, 60%)) in MeOH was added methyl 3-(6-methoxypyridin-2-yl)propanoate (500 mg, 2.56 mmol) in MeOH (total volume of MeOH is 15 mL). The reaction mixture was stirred overnight at room temperature. The reaction was diluted with EtOAc and a small amount of water. The organic layer was washed with brine and dried (Na₂SO₄). Evaporation provided ˜450 mg of product which was used without further purification.

(d) 3-(6-Methoxypyridin-2-yl)-N′-((8-methylquinolin-6-yl)methylene)-propanehydrazide: To a stirred solution of 3-(6-methoxypyridin-2-yl)propanehydrazide (200 mg, 1.02 mmol) in MeOH was added 8-methylquinoline-6-carbaldehyde (175 mg, 1.02 mmol) and the reaction mixture was stirred overnight at room temperature. The product was isolated by filtration providing the title compound as a colorless solid (151 mg, 42%). LCMS m/z 349 (M+H), t_{R}=8.9 min. (Method A).

### Example 27

### 3-(3,4-Dimethyl-1H-indol-1-yl)-N′-((8-methoxyquinolin-6-yl)methylene)propanehydrazide

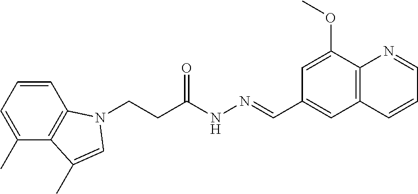

The title compound was prepared from 3-(3,4-dimethyl-1H-indol-1-yl)propanehydrazide (105 mg, 0.45 mmol) and 8-methoxyquinoline-6-carbaldehyde (85 mg, 0.45 mmol) in MeOH. The title compound was isolated by filtration yielding 125 mg as a colorless solid. LCMS m/z 401 (M+H), t_{R}=9.7 min. (Method A).

### Example 28

### trans-2-(3-Chlorophenyl)-N′-((8-chloroquinolin-6-yl)methylene)cyclopropanecarbohydrazide

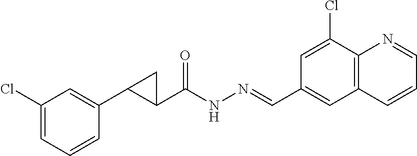

(a) trans-3-(3-Chlorophenyl)-N-methoxy-N-methylacrylamide: To a stirred solution of trans-3-chlorophenylcinnamic acid (250 mg, 1.4 mmol) in DCM at room temperature was added N,O-dimethylhydroxylamine hydrochloride (160 mg, 1.6 mmol), EDC (366 mg, 1.92 mmol), HOBT (258 mg, 1.92 mmol) and diisopropylethylamine (0.7 mL, 4.11 mmol). The reaction mixture was stirred overnight, then diluted with EtOAc. The organic phase was washed with water, brine and dried (Na₂SO₄). Evaporation provided a colorless viscous oil which was used directly in the next step.

(b) trans-2-(3-Chlorophenyl)-N-methoxy-N-methylcyclopropane-carboxamide: To a stirred solution of trimethylsulfoxonium iodide (726 mg, 3.29 mmol) in DMSO (3 mL) at room temperature was added NaH (79 mg, 1.98 mmol; 60% in mineral oil). After 1 hour, a DMSO solution (1.5 mL) of trans-3-(3-chlorophenyl)-N-methoxy-N-methylacrylamide (300 mg, 1.33 mmol) was added via dropwise addition. Stirring was continued for 1 hour at room temperature. The reaction was quenched with water and diluted with EtOAc. The organic phase was washed with water, brine and dried (Na₂SO₄). Evaporation provided a light yellow oil which was used without additional purification. (See K. E. Rodrigues, Tetrahedron Letters, 1991, 32 (10), 1275-1278.)

(c) 2-(3-Chlorophenyl)cyclopropanecarbohydrazide: Crude trans-2-(3-chlorophenyl)-N-methoxy-N-methylcyclopropanecarboxamide (˜1.3 mmol) and hydrazine hydrate (˜0.5 mL) in MeOH were heated at 90° C. for 3 hours (reaction proceeds very slowly at room temperature). The reaction was diluted with EtOAc and the organic phase was washed with brine and dried (Na₂SO₄). Evaporation provided the product as a semi-solid (280 mg).

(d) trans-2-(3-Chlorophenyl)-N′-((8-chloroquinolin-6-yl)methylene)-cyclopropanecarbohydrazide: To a stirred solution of trans-2-(3-chlorophenyl)cyclopropanecarbohydrazide (140 mg, 0.66 mmol) in MeOH (3 mL) at room temperature was added 8-chloroquinoline-6-carbaldehyde (127 mg, 0.66 mmol). The reaction mixture was stirred overnight and the product was isolated by filtration. The title compound was obtained as a colorless solid (130 mg, 51%). LCMS m/z 384 (M+H), t_{R}=10.8 min. (Method A).

### Example 29

### trans-2-(3-Chlorophenyl)-N′-((8-methylquinolin-6-yl)methylene)cyclopropanecarbohydrazide

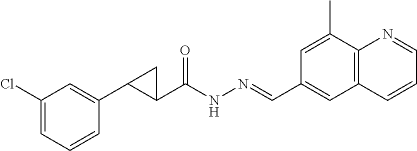

The title compound was prepared according to the method of Example 28(d) using 8-methylquinoline-6-carbaldehyde in place of 8-chloroquinoline-6-carbaldehyde. The product was obtained as a colorless solid (80 mg). LCMS m/z 364 (M+H), t_{R}=11.0 min. (Method A).

### Example 30

### trans-2-(3-Chlorophenyl)-N′-((8-methoxyquinolin-6-yl)methylene)cyclopropanecarbohydrazide

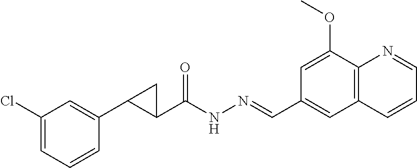

The title compound was prepared according to the method of Example 28(d) using 8-methoxyquinoline-6-carbaldehyde in place of 8-chloroquinoline-6-carbaldehyde. The product was obtained as an off-white solid (64 mg). LCMS m/z 380 (M+H), t_{R}=8.8 min. (Method A).

### Example 31

### trans-N′-((1,5-Naphthyridin-2-yl)methylene)-2-(3-chlorophenyl)cyclopropanecarbohydrazide

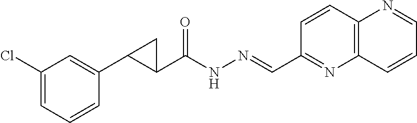

The title compound was prepared according to the method of Example 28(d) using 1,5-naphthyridine-2-carbaldehyde (prepared according to WO2006040052, Examples 1-2) in place of 8-chloroquinoline-6-carbaldehyde. The product was obtained as a colorless solid (125 mg). LCMS m/z 351 (M+H), t_{R}=8.7 min. (Method A).

### Example 32

### 3-(3-Methyl-1H-indol-1-yl)-N′-(quinoxalin-6-ylmethylene)propanehydrazide

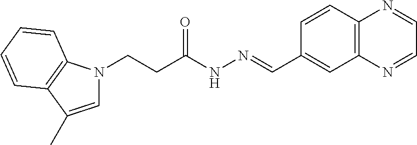

The title compound was prepared from quinoxaline-6-carbaldehyde (prepared from commercially available quinoxaline-6-carboxylic acid) and 3-(3-methyl-1H-indol-1-yl)propanehydrazide by methods already described herein. LCMS m/z 358 (M+H), t_{R}=3.1 min. (Method B).

### Example 33

### N′-((1,5-Naphthyridin-2-yl)methylene)-3-(3-methyl-1H-indol-1-yl)propanehydrazide

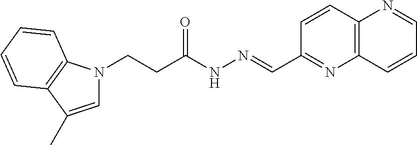

The title compound was prepared from 1,5-naphthyridine-2-carbaldehyde (prepared according to WO2006/040052, Examples 1-2) and 3-(3-methyl-1H-indol-1-yl)propanehydrazide by methods already described herein. LCMS m/z 358 (M+H), t_{R}=3.0 min. (Method B).

### Example 34

### N′-(Cinnolin-6-ylmethylene)-3-(3-methyl-1H-indol-1-yl)propanehydrazide

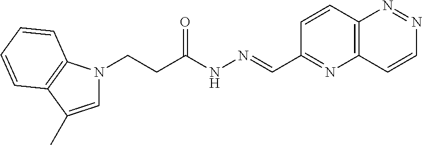

The title compound was prepared from cinnoline-6-carbaldehyde (prepared according to Example 3 of WO2007/038331) and 3-(3-methyl-1H-indol-1-yl)propanehydrazide by methods already described herein. LCMS m/z 358 (M+H), t_{R}=3.0 min. (Method B).

### Example 35

### 2-(3-Chlorophenylthio)-N′-((8-methylquinolin-6-yl)methylene)acetohydrazide

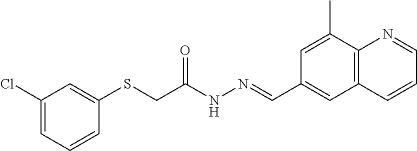

(a) Ethyl 2-(3-chlorophenylthio)acetate: To a stirred solution of 3-chlorobenzenethiol (0.5 g, 3.45 mmol) in CH₃CN (7 mL) was added ethyl 2-bromoacetate (0.4 mL, 3.45 mmol) and K₂CO₃ (0.95 g, 6.91 mmol). The reaction mixture was heated to 50° C. and stirred for 24 hours by which time the reaction mixture had become a white slurry. The reaction was diluted with EtOAc and washed with water and brine. The organic layer was dried (Na₂SO₄), filtered and concentrated. The product (0.75 g, 94%) was used without further purification.

(b) 2-(3-Chlorophenylthio)acetohydrazide: Ethyl 2-(3-chlorophenylthio)acetate (0.75 g, 3.25 mmol) was dissolved in MeOH (10 mL) and was added dropwise to a stirred solution of hydrazine hydrate (60%, 1.04 mL, 32.5 mmol) in MeOH (5 mL). The reaction mixture was heated at 70° C. for 1 hour. The reaction was diluted with EtOAc and washed with water (2×50 mL). The organic layer was dried (Na₂SO₄), filtered and concentrated. The product (0.70 g, 98%; white semi-solid) was used without further purification.

(c) 2-(3-Chlorophenylthio)-N′-((8-methylquinolin-6-yl)methylene)-acetohydrazide: To a stirred suspension of 2-(3-chlorophenylthio)-acetohydrazide (100 mg, 0.461 mmol) in absolute MeOH (5 mL) was added 8-methylquinoline-6-carbaldehyde (79 mg, 0.461 mmol). The reaction mixture was stirred overnight at room temperature and the resulting solid material was collected by filtration. The title compound was obtained as a colorless solid (100 mg, 59%). LCMS m/z 370 (M+H), t_{R}=10.2 min. (Method A).

### Example 36

### trans-N′-(3,4-Dimethoxybenzylidene)-2-(quinolin-2-yl)cyclopropanecarbohydrazide

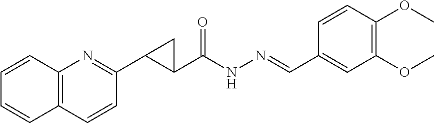

(a) (E)-N-methoxy-N-methyl-3-(quinolin-2-yl)acrylamide: Quinoline-2-carbaldehyde (2.5 g, 15.9 mmol) and N-methoxy-N-methyl-(triphenylphosphoranylidene)acetamide (4.6 g, 15.9 mmol) were mixed in DCM (40 mL) and stirred at room temperature for 1 hour. The solvent was evaporated and the residue was purified on silica gel to give the desired product (3.5 g).

(b) N-Methoxy-N-methyl-2-(quinolin-2-yl)cyclopropanecarboxamide: Trimethylsulfoxonium iodide (4.8 g, 21.68 mmol) in DMSO (30 mL) was treated with NaH (870 mg, 21.68 mmol) via portion-wise addition at room temperature. The reaction mixture was stirred at room temperature for 1 hour and then cooled to 0° C. (E)-N-methoxy-N-methyl-3-(quinolin-2-yl)acrylamide (3.5 g, 14.45 mmol) in DMSO (15 mL) was added and the reaction solution was warmed to 10° C. for 20 min. The reaction was diluted with EtOAc and water. The organic phase was washed with brine and concentrated to give an oil (3 g) which was used directly in the next step.

(c) 2-(Quinolin-2-yl)cyclopropanecarbohydrazide: N-Methoxy-N-methyl-2-(quinolin-2-yl)cyclopropanecarboxamide from Example 39(b) was dissolved in hydrazine (15 mL) and ethanol (15 mL). The solution was heated at 90° C. for 3 hours. The solution was concentrated in vacuo and the residue was triturated with petroleum ether to give a solid (3 g).

(d) trans-N′-(3,4-Dimethoxybenzylidene)-2-(quinolin-2-yl)cyclopropane-carbohydrazide: 2-(Quinolin-2-yl)cyclopropane-carbohydrazide (3 g, 13.2 mmol) and 3,4-dimethoxybenzaldehyde (4.4 g, 26.4 mmol) were mixed in ethanol (20 mL) and heated at 90° C. overnight. The solvent was evaporated and the residue was triturated with 20% petroleum ether/EtOAc to give the title compound (1.2 g). LCMS m/z 376 (M+H), t_{R}=3.4 min. (Method D).

### Example 37

### trans-N′-(3,4-Dimethoxybenzylidene)-2-(6-methylpyridin-2-yl)cyclopropanecarbohydrazide

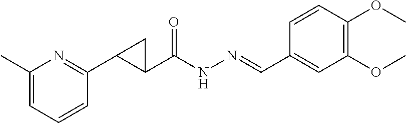

The title compound was prepared according to the method of Example 36 using 6-methylpicolinaldehyde in place of quinoline-2-carbaldehyde. LCMS m/z 340 (M+H), t_{R}=3.2 min. (Method D).

### Example 38

### trans-N′-(3,4-Dimethoxybenzylidene)-2-(quinolin-3-yl)cyclopropanecarbohydrazide

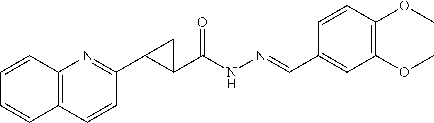

(a) (E)-N-Methoxy-N-methyl-3-(quinolin-3-yl)acrylamide: To a solution of 3-bromoquinoline (10 g, 48.1 mmol) in DMF (100 mL) was added N-methoxy-N-methylacrylamide (8.3 g, 72.1 mmol) and N₂ was bubbled through the solution for 10 min. (MeO)₃P (0.6 g, 4.8 mmol), Pd(OAc)₂ (0.5 g, 2.4 mmol) and DIPEA (9.3 g, 72.1 mmol) were added and the mixture was heated at 100° C. for 1 hour. The reaction mixture was cooled to room temperature and diluted with water (200 mL) and extracted with EtOAc (100 mL). The organic layer was washed with brine and dried (Na₂SO₄). The solvent was reduced in volume and the product was precipitated by the addition of EtOAc/petroleum ether (7.7 g).

(b) N-Methoxy-N-methyl-2-(quinolin-3-yl)cyclopropanecarboxamide: Trimethylsulfoxonium iodide (13.3 g, 60.26 mmol) in DMSO (100 mL) was treated with NaH (2.4 g, 60.26 mmol) via portion-wise addition at room temperature. The reaction mixture was stirred at room temperature for 1 hour and then cooled to 0° C. (E)-N-Methoxy-N-methyl-3-(quinolin-3-yl)acrylamide (7.3 g, 30.13 mmol) in DMSO (30 mL) was added and the reaction solution was warmed to 10° C. for 20 min. The reaction was diluted with EtOAc and water and the organic phase was washed with brine and concentrated. Purification on silica gel provided the desired product (3.7 g).

(c) 2-(Quinolin-3-yl)cyclopropanecarbohydrazide: N-Methoxy-N-methyl-2-(quinolin-3-yl)cyclopropanecarboxamide (3 g, 11.7 mmol) was dissolved in hydrazine (20 mL) and ethanol (20 mL). The solution was heated at 90° C. for 3 hours. The solution was concentrated in vacuo and the residue was triturated with petroleum ether to give a solid (1.2 g).

(d) trans-N′-(3,4-Dimethoxybenzylidene)-2-(quinolin-3-yl)cyclopropanecarbohydrazide: 2-(Quinolin-3-yl)cyclopropanecarbohydrazide (1.2 g, 5.28 mmol) and 3,4-dimethoxybenzaldehyde (1.14 mg, 6.86 mmol) were mixed in ethanol (10 mL) and heated at 90° C. overnight. The solvent was evaporated and the residue was triturated with 10% petroleum ether/EtOAc to give the title compound (900 mg). LCMS m/z 376 (M+H), t_{R}=3.6 min. (Method D).

### Example 39

### N′-(3,4-dimethoxybenzylidene)-3-(quinolin-3-yl)propanehydrazide

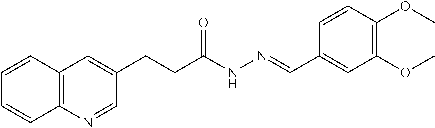

(a) N-Methoxy-N-methyl-3-(quinolin-3-yl)propanamide: (E)-N-Methoxy-N-methyl-3-(quinolin-3-yl)acrylamide (1.5 g, 6.2 mmol) was hydrogenated under an atmosphere of H₂ (1 MPa) in a pressure reactor using 5% Pd/C (150 mg) catalyst. After 3 hours, the reaction mixture was filtered and the product was purified on silica gel providing the desired product (500 mg).

(b) 3-(Quinolin-3-yl)propanehydrazide: N-Methoxy-N-methyl-3-(quinolin-3-yl)propanamide (500 mg, 2.05 mmol) was dissolved in ethanol (5 mL) and hydrazine (5 mL) and refluxed for 3 hours. The reaction was concentrated to dryness and the crude product was used directly in the next step without further purification.

(c) N′-(3,4-dimethoxybenzylidene)-3-(quinolin-3-yl)propanehydrazide: The crude material obtained in Example 39(b) was dissolved in ethanol and treated with 3,4-dimethoxybenzaldehyde (463 mg, 2.8 mmol) and refluxed for 3 hours. The solvent was removed and the product was purified on silica gel providing the title compound (550 mg). LCMS m/z 364 (M+H), t_{R}=3.4 min. (Method D).

### Example 40

### trans-2-(4-Fluorophenyl)-N′-((8-methylquinolin-6-yl)methylene)cyclopropanecarbohydrazide

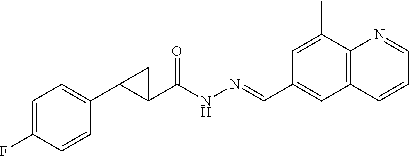

The title compound was prepared by similar methods to that of Example 36 using 4-fluorobenzaldehyde and 8-methylquinoline-6-carbaldehyde. LCMS m/z 348 (M+H), t_{R}=3.9 min. (Method D).

### Example 41

### trans-2-(6-Methoxypyridin-3-yl)-N′-((8-methylquinolin-6-yl)methylene)cyclopropanecarbohydrazide

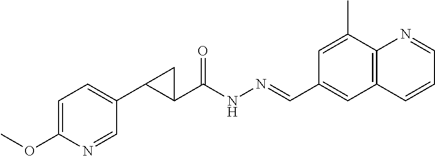

The title compound was prepared by similar methods to that of Example 36 using 6-methoxynicotinaldehyde and 8-methylquinoline-6-carbaldehyde. LCMS m/z 361 (M+H), t_{R}=3.6 min. (Method D).

### Example 42

### trans-2-(6-Methoxypyridin-2-yl)-N′-((8-methylquinolin-6-yl)methylene)cyclopropanecarbohydrazide

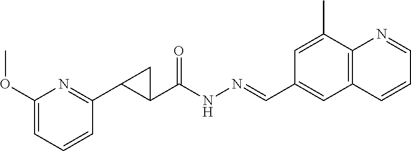

The title compound was prepared by similar methods to that of Example 36 using 6-methoxypicolinaldehyde and 8-methylquinoline-6-carbaldehyde. LCMS m/z 361 (M+H), t_{R}=3.9 min. (Method D).

### Example 43

### trans-2-(5-Chloropyridin-2-yl)-N′-((8-methylquinolin-6-yl)methylene)cyclopropanecarbohydrazide

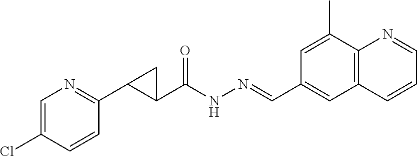

(a) 5-Chloropicolinonitrile: A mixture of 2-bromo-5-chloropyridine (4 g, 20.8 mmol) and CuCN (2.8 g, 31.18 mmol) in pyridine (40 mL) was heated at 130° C. for 4 hours. After cooling to room temperature the reaction was diluted with ether (50 mL) and water (50 mL). The ether layer was washed with water (3×) and brine. Concentration gave the crude solid which was used directly in the next step.

(b) 5-Chloropicolinic acid: The crude 5-chloropicolinonitrile from step (a) was dissolved in ethanol (20 mL) and 10% NaOH (20 mL) and heated at 90° C. for 1 hour. The reaction mixture was cooled to room temperature and adjusted to pH 3 with 2N HCl. The reaction solution was concentrated in vacuo and the residue was suspended in 10% methanol/DCM (50 mL). The solid was filtered and washed twice with 10% methanol/DCM (20 mL). The combined filtrate was concentrated to give crude product (1.7 g).

(c) 5-Chloro-N-methoxy-N-methylpicolinamide: To a stirred solution of 5-chloropicolinic acid (1.7 g, 10.79 mmol) in DMF (30 mL) was added BOP (5.7 g, 12.95 mmol), DIPEA (5.6 mL, 32.37) and N,O-dimethylhydroxylamine hydrochloride (1.6 g, 16.19 mmol). The reaction mixture was stirred at room temperature overnight. The reaction was diluted with EtOAc (50 mL) and water (60 mL) and the organic phase was washed with brine and concentrated to give crude product (2 g).

(d) 5-Chloropicolinaldehyde: To a stirred solution of crude 5-chloro-N-methoxy-N-methylpicolinamide from step (c) in THF (20 mL) at −50° C. was added LiAlH₄ (365 mg, 9.47 mmol) in portions. Stirring at −50° C. was continued for 30 min. The reaction was quenched by water, the solid was removed by filtration and the filtrate was concentrated to give crude product (1.2 g) which was used directly in the next step.

(e) trans-2-(5-Chloropyridin-2-yl)-N′-((8-methylquinolin-6-yl)methylene)-cyclopropanecarbohydrazide: The title compound was prepared by similar methods to that of Example 36 using 5-chloropicolinaldehyde from step (d) and 8-methylquinoline-6-carbaldehyde. LCMS m/z 365 (M+H), t_{R}=3.8 min. (Method D).

### Example 44

### trans-N′-((8-Fluoroquinolin-6-yl)methylene)-2-phenylcyclopropanecarbohydrazide

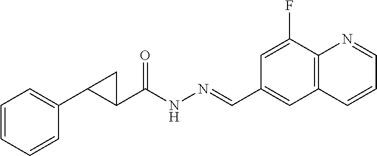

(a) 4-Amino-2-fluorobenzoic acid: 2-Fluoro-4-nitrobenzoic acid (5 g, 27.0 mmol) was dissolved in methanol (50 mL) and hydrogenated under an atmosphere of H₂ (balloon) in the presence of 10% Pd/C (100 mg) overnight. The catalyst was filtered through a pad of Celite and the filtrate was concentrated to give the desired product (4.3 g, 100%).

(b) 8-Fluoroquinoline-6-carboxylic acid: 4-Amino-2-fluorobenzoic acid (4.3 g, 27.0 mmol), sodium 3-nitrobenzenesulfonate (7.2 g, 32.0 mmol), 98% H₂SO₄ (15 mL) and water (6 mL) were heated to 120° C. To this was slowly added glycerol (7.1 g, 80.10 mmol). Following the addition, the reaction mixture was heated at 130° C. for 1.5 hours and then cooled to room temperature. The reaction mixture was poured onto crushed ice (150 g) and neutralized with NH₄OH to pH 6˜7. The aqueous mixture was extracted with 20% methanol/DCM (200 mL×2). Evaporation of the solvent gave crude product (5 g).

(c) 8-Fluoro-N-methoxy-N-methylquinoline-6-carboxamide: Crude 8-fluoroquinoline-6-carboxylic acid from step (b) (5 g) was suspended in DMF (50 mL) and treated with BOP (11.6 g, 26.18 mmol), DIPEA (13.7 mL, 78.53 mmol) and N,O-dimethylhydroxylamine hydrochloride (2.8 g, 28.80 mmol). The mixture was stirred at room temperature overnight. The reaction was diluted with EtOAc (150 mL) and water (150 mL) and the organic phase was washed with brine. Evaporation provided the desired product (850 mg) which was then used directly in the next step.

(d) 8-Fluoroquinoline-6-carbaldehyde: To a stirred solution of crude 8-fluoro-N-methoxy-N-methylquinoline-6-carboxamide from step (c) (850 mg) in THF (10 mL) at −40° C. was added LiAlH₄ (140 mg, 2.9 mmol) in portions. The mixture was maintained at −40° C. for 3 hours and then quenched with water. The solid was removed by filtration and the filtrate was concentrated to give crude aldehyde product (650 mg).

(e) trans-N′-((8-Fluoroquinolin-6-yl)methylene)-2-phenylcyclopropanecarbohydrazide: The crude aldehyde from step (d) (650 mg) and 2-phenylcyclopropanecarbohydrazide (600 mg, 3.4 mmol) were mixed in ethanol (10 mL) and refluxed for 3 hours. The solvent was evaporated and the product was purified on silica gel providing the title compound (780 mg). LCMS m/z 334 (M+H), t_{R}=4.1 min. (Method D).

### Example 45

### N′-((3,4-Dihydro-2H-benzo[b][1,4]dioxepin-7-ylmethylene)-3-(3,4-dimethyl-1H-indol-1-yl)propanehydrazide

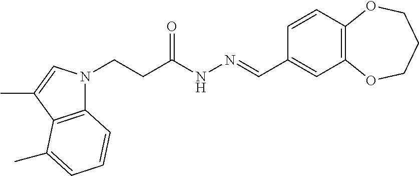

The title compound was prepared according the method of Example 15 using 3,4-dihydro-2H-benzo[b][1,4]dioxepine-7-carbaldehyde. LCMS m/z 392 (M+H), t_{R}=11.5 min. (Method A).

### Example 46

### N′-(3,4-Dimethoxybenzylidene)-3-(4-methoxy-3-methyl-1H-indol-1-yl)propanehydrazide

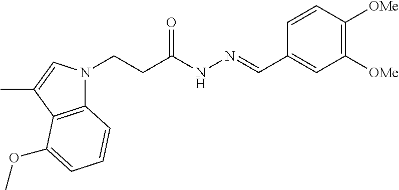

The title compound was prepared according to the method of Example 7 starting with 4-methoxyindole in place of 4-methylindole. LCMS m/z 396 (M+H), t_{R}=10.0 min. (Method A).

### Example 47

### 3-(4-Chloro-3-((dimethylamino)methyl)-1H-indol-1-yl)-N′-(3,4-dimethoxybenzylidene)propanehydrazide

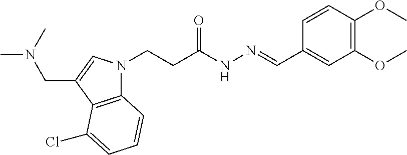

The title compound was prepared from 1-(4-chloro-1H-indol-3-yl)-N,N-dimethylmethanamine (prepared according to the method of Example 7(a) starting with 4-chloroindole) by methods already described. LCMS m/z 443 (M+H), t_{R}=7.0 min. (Method A).

### Example 48

### N′-(3,4-Dimethoxybenzylidene)-3-(1,7-dimethyl-1H-indol-3-yl)propanehydrazide

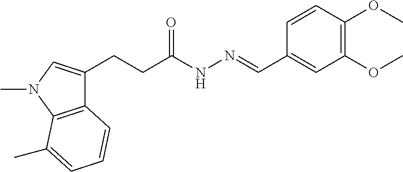

(a) (E)-Methyl 3-(7-methyl-1H-indol-3-yl)acrylate: To a stirred suspension of 7-methylindole-3-carboxaldehyde (796 mg, 5 mmol) in toluene (15 mL) and 1,4-dioxane (15 mL) was added methyl (triphenylphosphoranylidene)acetate (1.84 g, 5.5 mmol). The reaction mixture was heated to 85° C. and became homogeneous after 30 min. After 6.5 hours, LCMS indicated that some starting material was still present. An additional 900 mg of the ylide was added and the reaction mixture was maintained at 85° C. overnight. The reaction mixture was cooled and the solvent was evaporated. Purification on silica gel using EtOAc-hexane (0 to 40%) provided a pale yellow solid (1.02 g, 94%).

(b) Methyl 3-(7-methyl-1H-indol-3-yl)propanoate: (E)-Methyl 3-(7-methyl-1H-indol-3-yl)acrylate (1 g, 4.64 mmol) was hydrogenated under an atmosphere of H₂ (balloon) in MeOH (50 mL) using 10% Pd/C catalyst (200 mg). After 6 hours, the reaction mixture was filtered through a pad of Celite and evaporation provided a colorless solid (922 mg, 91%).

(c) N′-(3,4-Dimethoxybenzylidene)-3-(1,7-dimethyl-1H-indol-3-yl)-propanehydrazide: The compound was prepared following the method of Example 2 starting with methyl 3-(7-methyl-1H-indol-3-yl)propanoate from step (b). LCMS m/z 380 (M+H), t_{R}=9.9 min. (Method A).

### Example 49

### 3-(3-Chloro-4-methyl-1H-indol-1-yl)-N′-(3,4-dimethoxybenzylidene)propanehydrazide

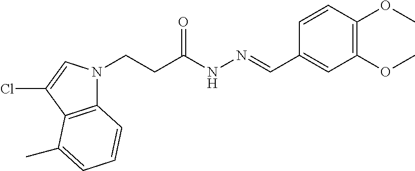

(a) 3-Chloro-4-methyl-1H-indole. To a stirred solution of 4-methylindole (0.6 g, 4.57 mmol) in MeOH (10 mL) at 0° C. was slowly added NCS (0.61 g, 4.57 mmol) over 10 min. The reaction mixture was stirred at 0° C. for 2 hours at which point silica gel was added directly to the flask and the reaction was concentrated under reduced pressure. Purification on silica gel using EtOAc-hexane (0 to 10%) provided a colorless solid (0.135 g, 18%).

(b) 3-(3-Chloro-4-methyl-1H-indol-1-yl)-N′-(3,4-dimethoxy-benzylidene)propanehydrazide: The compound was prepared following the method of Example 7 starting with 3-chloro-4-methyl-1H-indole from step (a). LCMS m/z 400 (M+H), t_{R}=10.8 min. (Method A).

### Example 50

### N′-(3,4-Dimethoxybenzylidene)-3-(4-(hydroxymethyl)-3-methyl-1H-indol-1-yl)propanehydrazide

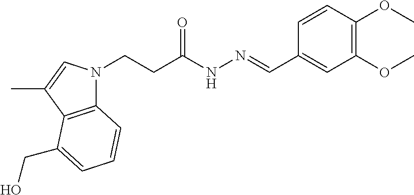

(a) Methyl 3-methyl-1H-indole-4-carboxylate. The compound was prepared according to the procedure of Example 7(a-b) starting with methyl 1H-indole-4-carboxylate.

(b) (3-Methyl-1H-indol-4-yl)methanol: Methyl 3-methyl-1H-indole-4-carboxylate (0.37 g, 1.96 mmol) was dissolved in THF (5 mL) and LiAlH₄ (0.81 mL, 1.96 mmol, 2.4 M THF) was added dropwise. The reaction mixture was stirred for 3 hours and quenched by the slow addition of water. The reaction was filtered through a pad of Celite and the filtrate was transferred to a separatory funnel using EtOAc. The organic layer was washed with water, dried (Na₂SO₄), filtered and concentrated to yield the desired product (0.28 g, 88%).

(c) N′-(3,4-Dimethoxybenzylidene)-3-(4-(hydroxymethyl)-3-methyl-1H-indol-1-yl)propanehydrazide: The compound was prepared from (3-methyl-1H-indol-4-yl)methanol by methods already described. LCMS m/z 396 (M+H), t_{R}=9.1 min. (Method A).

### Example 51

### 2-(3-Chlorophenylsulfonyl)-N′-(3,4-dimethoxybenzylidene)propanehydrazide

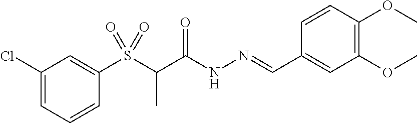

2-(3-Chlorophenylthio)-N′-(3,4-dimethoxybenzylidene)propane-hydrazide (75 mg, 0.198 mmol; Example 1) was dissolved in DCM (2 mL) and treated with mCPBA (100 mg, 0.594 mmol). The reaction mixture was stirred at room temperature for 12 hours and diluted with EtOAc and washed with water (2×50 mL). The organic layer was dried (Na₂SO₄), filtered and concentrated directly onto silica gel. Purification on silica gel using EtOAc-hexane (0 to 35%) provided the title compound as a colorless solid (60 mg, 74%). LCMS m/z 411 (M+H), t_{R}=9.0 min. (Method A).

### Example 52

### N′-((2-Methoxy-4-methylquinolin-6-yl)methylene)-3-(3-methyl-1H-indol-1-yl)propanehydrazide

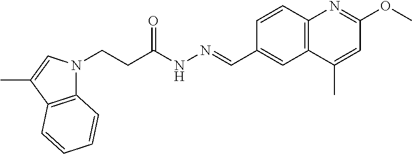

(a) N-(4-Bromophenyl)-3-oxobutanamide: 4-Bromoaniline (2.5 g, 14.53 mmol) and ethyl 3-oxobutanoate (4 mL) were placed in a microwave vial and subjected to numerous heating sessions in the microwave (150° C. for 20 min, 150° C. for 5 min and 160° C. for 15 min) while noting the continued presence of starting material after each run. The reaction was then diluted with EtOAc and washed with 1N HCl (3×20 mL) and water (2×20 mL). The organic layer was dried (Na₂SO₄), filtered and concentrated. The resulting yellow solid were triturated with 10% EtOAc-hexane and then filtered to yield the desired product as a tan solid (0.73 g, 20%).

(b) 6-Bromo-4-methylquinolin-2(1H)-one: N-(4-Bromophenyl)-3-oxobutanamide (0.73 g, 2.85 mmol) was dissolved in conc. H₂SO₄ (7 mL) and heated to 120° C. via oil bath for 1 hour. The reaction mixture was cooled to room temperature, transferred to a beaker containing ice and the resulting precipitates were filtered off. The desired product was isolated as a tan solid (0.4 g, 60%).

(c) 6-Bromo-2-chloro-4-methylquinoline: 6-Bromo-4-methylquinolin-2(1H)-one (0.13 g, 0.546 mmol) was placed in a microwave vial and treated with POCl₃ (0.4 mL, 3.78 mmol). The reaction was heated in the microwave at 100° C. for 5 min. The reaction was poured into a beaker containing ice, neutralized using sat. NaHCO₃ and then transferred to a separatory funnel using EtOAc. The organic layer was washed with water, dried (Na₂SO₄), filtered and concentrated to yield the desired product (0.12 g, 86%).

(d) 6-Bromo-2-methoxy-4-methylquinoline: 6-Bromo-2-chloro-4-methylquinoline (0.055 g, 0.215 mmol) was placed in a sealed tube and treated with NaOMe (0.29 mL, 0.537 mmol) and MeOH (1 mL). The reaction was capped and heated at 70° C. for 12 hours. The reaction mixture was cooled to room temperature, diluted with EtOAc and washed with water (2×20 mL). The organic layer was dried (Na₂SO₄), filtered and concentrated. The product was purified through a plug of silica eluting with 100% hexanes to remove baseline material. The desired product was isolated as a white solid (0.035 g, 65%).

(e) 2-Methoxy-4-methylquinoline-6-carbaldehyde: To a stirred solution of 6-bromo-2-methoxy-4-methylquinoline (0.035 g, 0.139 mmol) in THF (1 mL) at −78° C. was added n-BuLi (0.17 mL, 0.278 mmol) via dropwise addition. The reaction mixture was stirred for 5 min followed by the addition of DMF (32 uL, 0.417 mmol). The reaction was allowed to warm to 0° C. and 1N HCl (1 mL) was then added. The reaction was diluted with EtOAc and washed with water (2×20 mL). The organic layer was dried (Na₂SO₄), filtered and concentrated. The resulting product was isolated as a viscous yellow oil (0.025 g, 88%).

(f) N′-((2-Methoxy-4-methylquinolin-6-yl)methylene)-3-(3-methyl-1H-indol-1-yl)propanehydrazide: The title compound was prepared from 2-methoxy-4-methylquinoline-6-carbaldehyde by methods already described. LCMS m/z 401 (M+H), t_{R}=12.4 min. (Method A).

### Example 53

### 2-(3-Chlorophenylthio)-N′-((4,5-dimethoxypyridin-2-yl)methylene)propanehydrazide

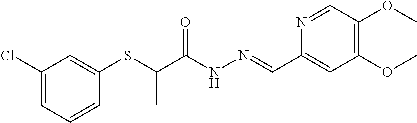

(a) 4,5-Dimethoxypicolinaldehyde: The compound was prepared according to methods described in O'Malley et. al., Org. Letters, 2006, 8(12), 2651-2652.

(b) 2-(3-Chlorophenylthio)-N′-((4,5-dimethoxypyridin-2-yl)-methylene)propanehydrazide: The title compound was prepared from 4,5-dimethoxypicolinaldehyde by methods already described. LCMS m/z 380 (M+H), t_{R}=9.2 min. (Method A).

### Examples 54-69

The following compounds in TABLE 1 were prepared following methods similar to those outlined above:

TABLE 1
Compounds of Examples 54-69

| | | LCMS, | LCMS |
|---|---|---|---|
| Example | Compound/Name | m/z, tg | Method |
| 54 | 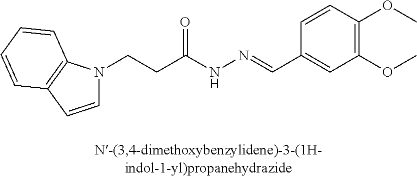 | 352, 8.8 min | A |
| 55 | 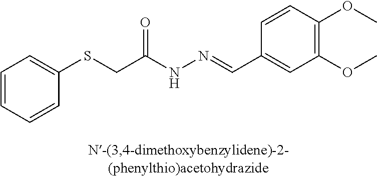 | | |
| 56 | 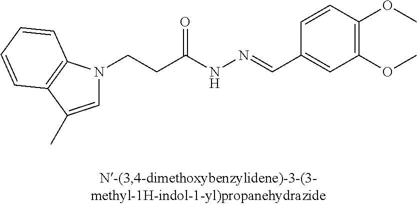 | 366, 10.2 min | A |
| 57 | 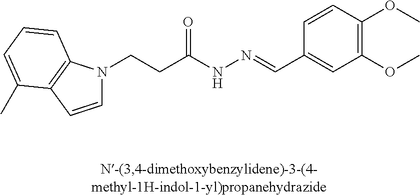 | 366, 9.9 min | A |
| 58 | 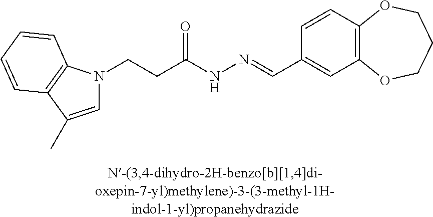 | 378, 3.6 min | E |
| 59 | 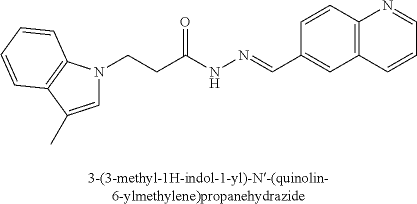 | 357, 3.5 min | E |
| 60 | 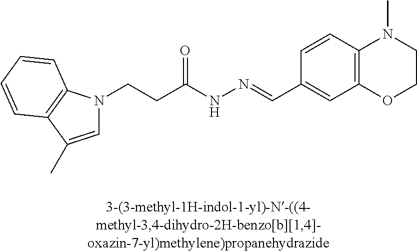 | 377, 3.5 min | E |
| 61 | 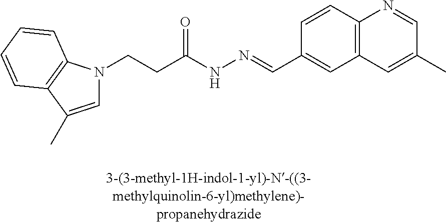 | 371, 4.4 min | B |
| 62 | 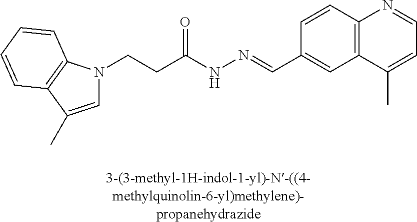 | 371, 2.6 min | E |
| 63 | 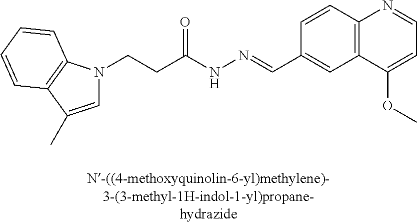 | 387, 2.3 min | E |
| 64 | 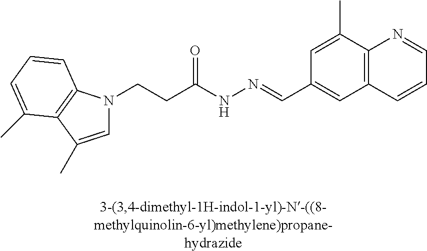 | 385, 11.2 min | A |
| 65 | 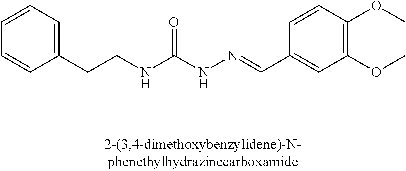 | 328, 3.0 min | E |
| 66 | 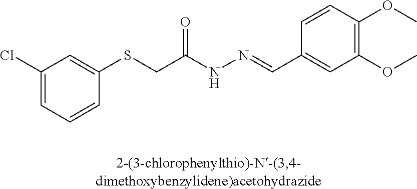 | 365, 1.9 min | E |
| 67 | 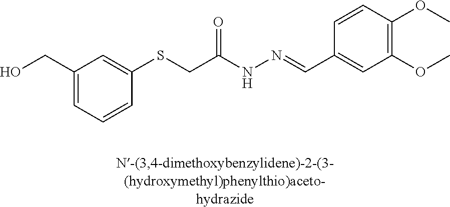 | 361, 2.8 min | B |
| 68 | 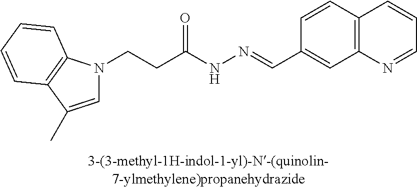 | 357, 2.1 min | E |
| 69 | 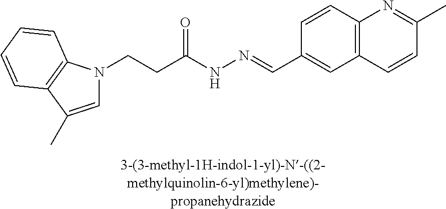 | 371, 10.3 min | A |

### Example 70

Representative compounds of the present invention have been tested in the FLIPR assay for TRPM5 inhibiting activity, which is described in detail above. Representative values are presented in TABLE 2.

TABLE 2
Evaluation of the tested compounds as TRPM5
inhibitors in a FLIPR-based in vitro assay for
membrane potential and calcium mobilization

| Example | hTrpM5 IC₅₀, uM |
|---|---|
| 1 | 0.055 |
| 2 | 0.051 |
| 3 | 0.030 |
| 4 | 0.50 |
| 5 | 0.051 |
| 6 | 0.037 |
| 7 | 0.0018 |
| 8 | 0.003 |
| 9 | 5.0 |
| 10 | 0.42 |
| 11 (E1) | >10 |
| 11 (E2) | 0.24 |
| 12 | 0.08 |
| 13 | 0.066 |
| 14 | 0.68 |
| 15 | 0.038 |
| 16 | 0.053 |
| 17 | 2.4 |
| 18 | 0.046 |
| 19 | 0.18 |
| 20 | 1.6 |
| 21 | 0.06 |
| 22 | 0.09 |
| 23 | 0.16 |
| 24 | 10 |
| 25 | 0.34 |
| 26 | 2 |
| 27 | 0.0014 |
| 28 | 0.65 |
| 29 | 0.8 |
| 30 | 0.045 |
| 31 | ~ 50 |
| 32 | 2.2 |
| 33 | 1.3 |
| 34 | 0.43 |
| 35 | 0.83 |
| 36 | 0.93 |
| 37 | 3.2 |
| 38 | 0.61 |
| 39 | 0.81 |
| 40 | 0.51 |
| 41 | 5.0 |
| 42 | 0.41 |
| 43 | 5.4 |
| 44 | 0.55 |
| 45 | 0.024 |
| 46 | 0.006 |
| 47 | 1.5 |
| 48 | 0.009 |
| 49 | 0.0013 |
| 50 | 0.26 |
| 51 | 1.7 |
| 52 | 2 |
| 53 | >30 |
| 54 | 0.17 |
| 55 | 0.66 |
| 56 | 0.017 |
| 57 | 0.012 |
| 58 | 0.18 |
| 59 | 0.34 |
| 60 | 0.75 |
| 61 | 0.21 |
| 62 | 0.37 |
| 63 | 0.12 |
| 64 | 0.005 |
| 65 | 0.2 |
| 66 | 0.04 |
| 67 | >30 |
| 68 | >30 |
| 69 | >30 |

### Example 71

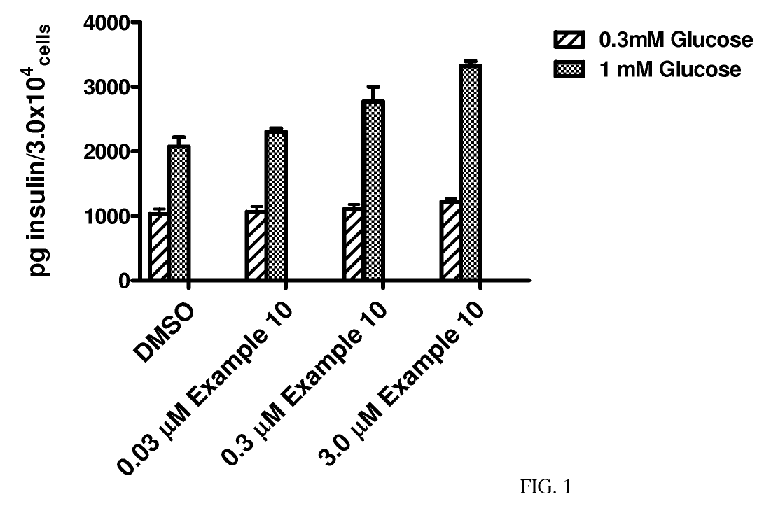

The compound of Example 10 was tested for insulin release enhancement in the insulinoma cell line Beta-TC-6 according to the procedure described in detail above (Assay 1). The results of this test are presented in FIG. 1.

FIG. 1 illustrates that the tested compound promotes insulin secretion in a dose-dependent and glucose-dependent manner in the insulinoma cell line Beta-TC-6. Insulin secretion by the tested compound does not occur in the presence of 0.3 mM glucose. In contrast, in the presence of 1 mM glucose there is a dose-dependent increase of insulin secretion promoted by the compound.

### Example 72

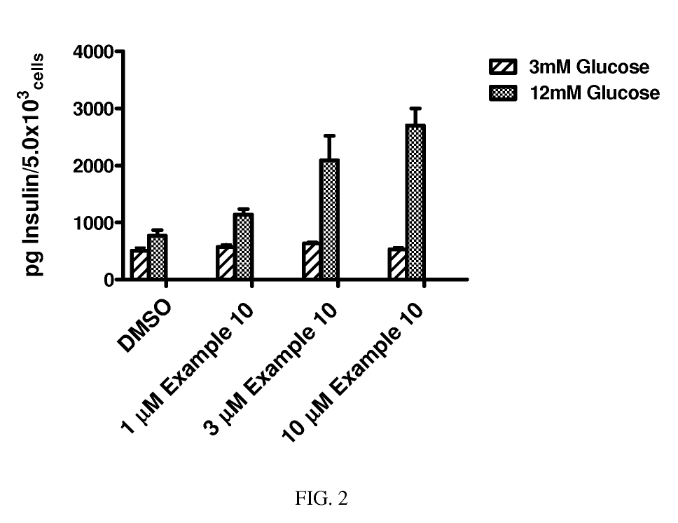

The compound of Example 10 was tested for insulin secretion in rat primary islets as described in detail above. The results of this test are presented in FIG. 2.

FIG. 2 illustrates that the tested compound promotes insulin secretion in a glucose-dependent and dose-dependent manner in isolated rat primary islets. In the presence of 3 mM glucose, insulin secretion is not observed by the tested compound even up to concentrations of 10 μM. In contrast, in the presence of 12 mM glucose, there is a significant dose-dependent increase in insulin secretion observed as compared to the DMSO control.

### Example 73

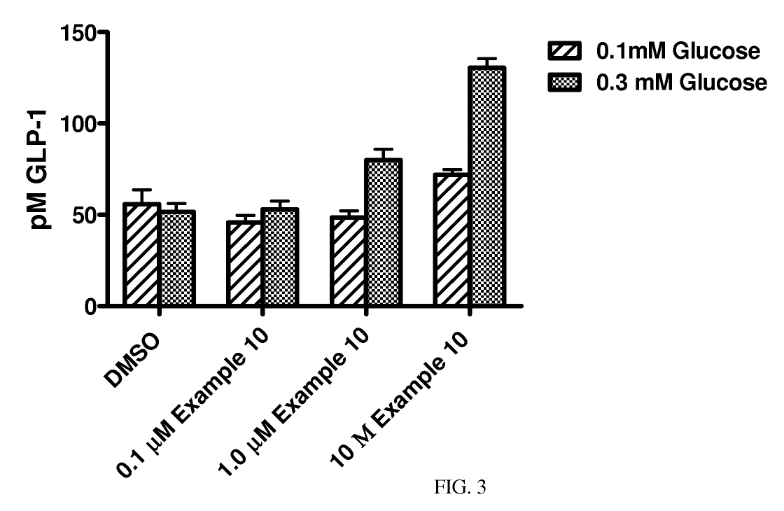

The compound of Example 10 was tested for GLP-1 secretion in the endochrine cell line GLUTag according to the procedure described in detail above (Assay 1). The results of this test are presented in FIG. 3.

FIG. 3 illustrates that the tested compound promotes GLP-1 secretion in a dose-dependent and glucose-dependent manner in the enteroendocrine cell line GLUTag. Secretion of GLP-1 by the tested compound is not observed in the presence of 0.1 mM glucose. In contrast, in the presence of 0.3 mM glucose, there is a dose-dependent increase of GLP-1 secretion promoted by the compound.

Having now fully described this invention, it will be understood by those of ordinary skill in the art that the same can be performed within a wide and equivalent range of conditions, formulations and other parameters without affecting the scope of the invention or any embodiment thereof. All patents, published patent applications, and publications cited herein are fully incorporated by reference herein in their entirety.

1. A compound having the Formula I: 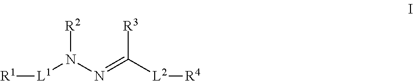
or a pharmaceutically acceptable salt or stereoisomer thereof, wherein:
R² and R³ are each independently hydrogen or alkyl;
R⁴ is aryl, a 6-membered heteroaryl having at least one nitrogen atom, or a 9-10-membered heteroaryl having at least one nitrogen atom, each of which is optionally substituted; or
R⁴ is phenyl, wherein two adjacent carbon atoms of the phenyl ring form a bridge —O—(CH₂)ᵣ—O— or —(NR²⁶)—(CH₂)ₛ—O— to form a fused bicyclic ring, wherein r and s are 1, 2, or 3, and R²⁶ is H or C₁₋₄ alkyl, and wherein the bicyclic ring is optionally substituted;
L² is absent; and
R¹-L¹- is selected from the following: 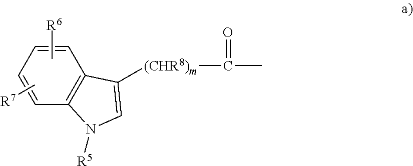
wherein
R⁵ is C₁₋₆ alkyl;
R⁶ and R⁷, which can be present in either ring of the indol-3-yl group, are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl;
R⁸ is independently hydrogen or C₁₋₆ alkyl; and
m is 1, 2, or 3;
b)
R^{1d}-cyclopropyl-C(═O)—, wherein
R^{1d} is optionally substituted phenyl or optionally substituted 6-10-membered heteroaryl having at least one nitrogen atom,
provided that 1) when R² and R³ are hydrogen, R⁴ is unsubstituted phenyl or phenyl substituted with other than one of arylalkoxy or heteroarylalkoxy, and R^{1d} is optionally substituted phenyl, then R^{1d} is substituted by at least one haloalkyl, or 2) when R^{1d} is optionally substituted phenyl, then R⁴ is other than optionally substituted isoquinolinyl;
c) R^{1a}—Z —(CHR¹²)ₙ—C(O)—; wherein
R^{1a} is optionally substituted aryl,
R¹² is independently hydrogen or C₁₋₆ alkyl,
Z is S, SO, or SO₂, and
n is 1, 2, or 3;
d) R^{1b}—(CHR¹³)ₚ—NH—C(O)—; wherein
R^{1b} is alkyl, cycloalkyl, aryl or heteroaryl, each of which is optionally substituted,
R¹³ is independently hydrogen or C₁₋₆ alkyl, and
p is 0, 1, 2, or 3; or
e) R^{1c}—(CHR¹⁴)_{q}—C(O)—, wherein
R^{1c} is optionally substituted indol-1-yl, optionally substituted pyrrolo[2,3-b]pyridin-1-yl, optionally substituted benzoimidazol-1-yl, optionally substituted quinolinyl, or optionally substituted pyridinyl;
R¹⁴ is independently hydrogen or C₁₋₆ alkyl; and
q is 1, 2, or 3

2. A compound having the Formula I: 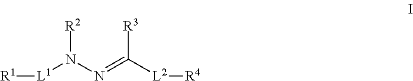
or a pharmaceutically acceptable salt or stereoisomer thereof, wherein:
R² and R³ are each independently hydrogen or alkyl;
R⁴ is aryl or a 9-10-membered heteroaryl having at least one nitrogen atom, each of which is optionally substituted;
L² is absent; and
R¹-L¹- is selected from the following: 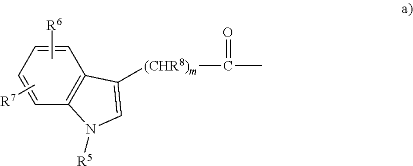
wherein
R⁵ is C₁₋₆ alkyl;
R⁶ and R⁷, which can be present in either ring of the indol-3-yl group, are each independently selected from the group consisting of hydrogen, halogen, alkyl, alkoxy, alkoxyalkyl, haloalkyl, haloalkoxy, hydroxyalkyl, aminoalkyl, hydroxy, nitro, cyano, amino, alkylamino, and dialkylamino;
R⁸ is independently hydrogen or C₁₋₆ alkyl; and
m is 1, 2, or 3; 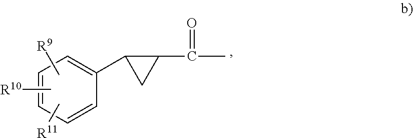
wherein
R⁹, R¹⁰, and R¹¹ are each independently selected from the group consisting of hydrogen, halogen, alkyl, alkoxy, alkoxyalkyl, haloalkyl, haloalkoxy, hydroxyalkyl, aminoalkyl, hydroxy, nitro, cyano, amino, alkylamino, and dialkylamino,
provided that when R² is hydrogen, then R⁹ is haloalkyl;
c) R^{1a}—S—(CHR¹²)ₙ—C(O)—; wherein
R^{1a} is optionally substituted aryl,
R¹² is independently hydrogen or C₁₋₆ alkyl, and
n is 1, 2, or 3;
d) R^{1b}—(CHR¹³)ₚ—NH—C(O)—; wherein
R^{1b} is alkyl, cycloalkyl, aryl or heteroaryl, each of which is optionally substituted,
R¹³ is independently hydrogen or C₁₋₆ alkyl, and
p is 0, 1, 2, or 3; or
e) R^{1c}—(CHR¹⁴)_{q}—C(O)—, wherein
R^{1c} is optionally substituted indol-1-yl;
R¹⁴ is independently hydrogen or C₁₋₆ alkyl; and
q is 1, 2, or 3

3. The compound of claim 1, wherein R¹-L¹- is 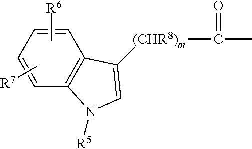
wherein
R⁵ is C₁₋₆ alkyl;
R⁶ and R⁷, which can be present in either ring of the indol-3-yl group, are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl;
R⁸ is independently hydrogen or C₁₋₆ alkyl; and
m is 1, 2, or 3.

4-9. (canceled)

10. The compound of claim 1, wherein R¹-L¹- is
R^{1d}-cyclopropyl-C(═O)—, wherein
R^{1d} is optionally substituted phenyl or optionally substituted 6-10-membered heteroaryl having at least one nitrogen atom,
provided that 1) when R² and R³ are hydrogen, R⁴ is unsubstituted phenyl or phenyl substituted with other than one of arylalkoxy or heteroarylalkoxy, and R^{1d} is optionally substituted phenyl, then R^{1d} is substituted by at least one haloalkyl, or 2) when R^{1d} is optionally substituted phenyl, then R⁴ is other than optionally substituted isoquinolinyl.

11. The compound of claim 10, wherein R^{1d} is an optionally substituted 6-10-membered heteroaryl having at least one nitrogen atom.

12. The compound of claim 11, wherein R^{1d} is an optionally substituted 6- or 9-10-membered heteroaryl having at least one nitrogen atom and R⁴ is optionally substituted aryl.

13. The compound of claim 1, wherein R² is hydrogen and R¹-L¹- is 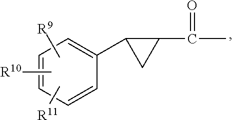
wherein
R⁹ is haloalkyl, and R¹⁰ and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

14. The compound of claim 13, wherein R⁹ is halo(C₁₋₆)alkyl, and R¹⁰ and R¹¹ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino.

15. The compound of claim 14, wherein R⁹ is trifluoromethyl.

16-17. (canceled)

18. The compound of claim 10, wherein R^{1d} is optionally substituted phenyl and R⁴ is substituted phenyl, wherein at least one substituent is arylalkyloxy or heteroarylalkyloxy, wherein the aryl and heteroaryl groups can be optionally substituted.

19. The compound of claim 1, wherein R² is alkyl and R¹-L¹- is 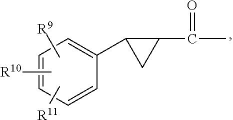
wherein
R⁹, R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

20. The compound of claim 19, wherein R⁹, R¹⁰ and R¹¹ are each independently selected from the group consisting of hydrogen, halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino.

21-24. (canceled)

25. The compound of claim 1, wherein R¹-L¹- is
R^{1a}—Z—(CHR¹²)ₙ—C(O)—;
wherein
R^{1a} is optionally substituted aryl,
R¹² is independently hydrogen or C₁₋₆ alkyl,
Z is S, SO, or SO₂, and
n is 1, 2, or 3.

26-27. (canceled)

28. The compound of claim 25, wherein R^{1a} is phenyl, naphthyl or biphenyl, each of which is optionally substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino.

29-33. (canceled)

34. The compound of claim 1, wherein R¹-L¹- is
R^{1b}—(CHR¹³)ₚ—NH—C(O)—;
wherein
R^{1b} is alkyl, cycloalkyl, aryl or heteroaryl, each of which is optionally substituted,
R¹³ is independently hydrogen or C₁₋₆ alkyl, and
p is 0, 1, 2, or 3.

35-36. (canceled)

37. The compound of claim 34, wherein R^{1b} is unsubstituted C₁₋₆ alkyl or unsubstituted C₅₋₆ cycloalkyl.

38. The compound of claim 34, wherein R^{1b} is phenyl, naphthyl or biphenyl, each of which is optionally substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino.

39-40. (canceled)

41. The compound of claim 34, wherein R^{1b} is unsubstituted heteroaryl or heteroaryl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino.

42-43. (canceled)

44. The compound of claim 1, wherein R¹-L¹- is
R^{1c}—(CHR¹⁴)_{q}—C(O)—, wherein
R^{1c} is optionally substituted indol-1-yl, optionally substituted pyrrolo[2,3-b]pyridin-1-yl, optionally substituted benzoimidazol-1-yl, optionally substituted quinolinyl, or optionally substituted pyridinyl;
R¹⁴ is independently hydrogen or C₁₋₆ alkyl; and
q is 1, 2, or 3.

45-46. (canceled)

47. The compound of claim 44, wherein R^{1c} is unsubstituted indol-1-yl or indol-1-yl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

48. The compound of claim 47, wherein R^{1c} is indol-1-yl substituted with 1, 2, or 3 substituents each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino.

49-50. (canceled)

51. The compound of claim 44, wherein R^{1c} is unsubstituted pyrrolo[2,3-b]pyridin-1-yl or pyrrolo[2,3-b]pyridin-1-yl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

52. The compound of claim 44, wherein R^{1c} is unsubstituted benzoimidazol-1-yl or benzoimidazol-1-yl substituted with 1, 2, or 3 substituents each independently selected from the group consisting of halogen, alkyl, cyclo alkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

53. The compound of claim 44, wherein R^{1c} is unsubstituted quinolinyl or quinolinyl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

54. The compound of claim 44, wherein R^{1c} is unsubstituted pyridinyl or pyridinyl substituted with 1, 2, or 3 substituents each independently selected from the group consisting of halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl.

55-56. (canceled)

57. The compound of claim 1, wherein R² is hydrogen.

58. The compound of claim 1, wherein R² is C₁₋₄ alkyl.

59-60. (canceled)

61. The compound of claim 1, wherein R⁴ is optionally substituted phenyl.

62. The compound of claim 61, wherein R⁴ is phenyl substituted with 1, 2, or 3 substituents each independently selected from the group consisting of halogen, alkyl, cycloalkyl, alkoxy, alkoxyalkyl, haloalkyl, haloalkoxy, hydroxyalkyl, hydroxyalkyloxy, alkoxyalkyloxy, carboxyalkoxy, alkoxycarbonylalkoxy, aminoalkyl, hydroxy, nitro, cyano, amino, alkylamino, dialkylamino, (alkylamino)alkyl, (dialkylamino)alkyl, optionally substituted arylalkyloxy, and optionally substituted heteroarylalkyloxy.

63-65. (canceled)

66. The compound of claim 1, wherein R⁴ is a 6-membered heteroaryl having at least one nitrogen atom, wherein the heteroaryl is optionally substituted with 1, 2, or 3 substituents each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino.

67. The compound of claim 1, wherein R⁴ is a 9-10-membered heteroaryl having at least one nitrogen atom, wherein the heteroaryl is optionally substituted with 1, 2, or 3 substituents each independently selected from the group consisting of halogen, C₁₋₆ alkyl, C₃₋₆ cycloalkyl, C₁₋₆ alkoxy, C₁₋₆ alkoxy(C₁₋₆)alkyl, halo(C₁₋₆)alkyl, halo(C₁₋₆)alkoxy, hydroxy(C₁₋₆)alkyl, amino(C₁₋₆)alkyl, hydroxy, nitro, cyano, amino, C₁₋₆ alkylamino, and di(C₁₋₄)alkylamino.

68. (canceled)

69. The compound of claim 67, wherein the heteroaryl is selected from the group consisting of quinolinyl, quinoxalinyl, benzothiazolyl, pyrido[3,4-b]pyridinyl, pyrido[3,2-b]pyridinyl, pyrido[4,3-b]pyridinyl, 1,5-naphthyridinyl, and cinnolinyl.

70. The compound of claim 1, wherein R⁴ is phenyl, wherein two adjacent carbon atoms of the phenyl ring form a bridge —O—(CH₂)ᵣ—O— or —(NR²⁶)—(CH₂)ₛ—O— to form a fused bicyclic ring, wherein r and s are 1, 2, or 3, and R²⁶ is H or C₁₋₄ alkyl, and wherein the bicyclic ring is optionally substituted.

71. The compound of claim 1 having the Formula II: 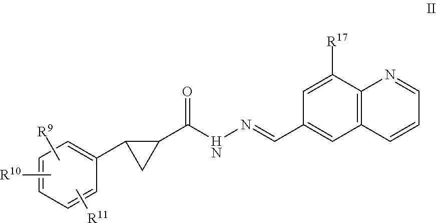
or a pharmaceutically acceptable salt or a stereoisomer thereof, wherein R⁹, R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino, and R¹⁷ is C₁₋₄ alkyl, C₃₋₆ cycloalkyl, C₁₋₄ alkoxy, halogen, halo(C₁₋₄)alkyl, or halo(C₁₋₄)alkoxy.

72. The compound of claim 1 having the Formula III: 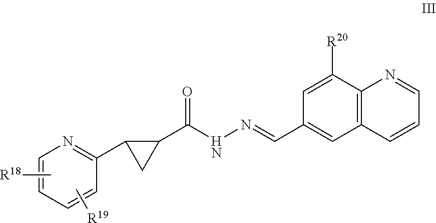
or a pharmaceutically acceptable salt or a stereoisomer thereof, wherein R¹⁸ and R¹⁹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and R²⁰ is C₁₋₄ alkyl, C₃₋₆ cycloalkyl, C₁₋₄ alkoxy, halogen, halo(C₁₋₄)alkyl, or halo(C₁₋₄)alkoxy.

73. The compound of claim 1 having the Formula IV: 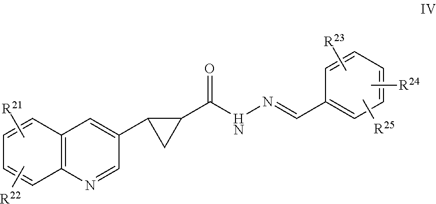
or a pharmaceutically acceptable salt or a stereoisomer thereof, wherein R²¹ and R²² are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, dialkylamino, (alkylamino)alkyl, and (dialkylamino)alkyl, and R²³, R²⁴, and R²⁵ are each independently selected from the group consisting of hydrogen, halogen, alkyl, cycloalkyl, alkoxy, alkoxyalkyl, haloalkyl, haloalkoxy, hydroxyalkyl, hydroxyalkyloxy, alkoxyalkyloxy, carboxyalkoxy, alkoxycarbonylalkoxy, aminoalkyl, hydroxy, nitro, cyano, amino, alkylamino, dialkylamino, (alkylamino)alkyl, (dialkylamino)alkyl, optionally substituted arylalkyloxy, and optionally substituted heteroarylalkyloxy.

74. The compound of claim 1 having the Formula V: 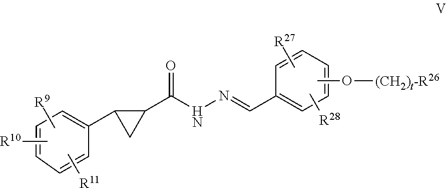
or a pharmaceutically acceptable salt or a stereoisomer thereof, wherein R⁹, R¹⁰, and R¹¹ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino, R²⁶ is optionally substituted phenyl or optionally substituted heteroaryl, R²⁷ and R²⁸ are each independently selected from the group consisting of hydrogen, halogen, alkyl, cycloalkyl, alkoxy, alkoxyalkyl, haloalkyl, haloalkoxy, hydroxyalkyl, aminoalkyl, hydroxy, nitro, cyano, amino, alkylamino, and dialkylamino, and t is 1, 2, or 3.

75. The compound of claim 1, wherein said compound is:
2-(3-chlorophenylthio)-N′-(3,4-dimethoxybenzylidene)propane-hydrazide;
N′-(3,4-dimethoxybenzylidene)-3-(1-methyl-1H-indol-3-yl)propane-hydrazide;
3-(3-methyl-1H-indol-1-yl)-N′-((8-methylquinolin-6-yl)methylene)-propanehydrazide;
N′-(4-(3-hydroxypropoxy)-3-methoxybenzylidene)-3-(3-methyl-1H-indol-yl)propanehydrazide;
N-(4-chlorophenethyl)-2-(3,4-dimethoxybenzylidene)hydrazine-carboxamide;
N′-(3,4-dimethoxybenzylidene)-2-(3-trifluoromethyl)phenyl)-cyclopropanecarbohydrazide;
N′-(3,4-dimethoxybenzylidene)-3-(3,4-dimethyl-1H-indol-1-yl)-propanehydrazide;
3-(3,4-dimethyl-1H-indol-1-yl)-N′-(2-fluoro-4,5-dimethoxy-benzylidene)propanehydrazide;
N′-((8-methylquinolin-6-yl)methylene)-2-phenylcyclopropane-carbohydrazide;
N′-(4-(benzyloxy)-3-methoxybenzylidene)-3-(3,4-dimethyl-1H-indol-1-yl)propanehydrazide;
3-(3,4-dimethyl-1H-indol-1-yl)-N′-(3-methoxy-4-(pyridin-2-ylmethoxy)benzylidene)propanehydrazide;
N′-(3,4-dimethoxybenzylidene)-3-(3-methyl-1H-pyrrolo[2,3-b]pyridin-1-yl)propanehydrazide;
N′-(3-chloro-4,5-dimethoxybenzylidene)-3-(3,4-dimethyl-1H-indol-1-yl)propanehydrazide;
3-(3,4-dimethyl-1H-indol-1-yl)-N′-(3-methoxy-4-(pyridin-3-ylmethoxy)benzylidene)propanehydrazide;
N′-(3,4-dimethoxybenzylidene)-3-(4-methyl-1H-benzo[d]imidazol-1-yl)propanehydrazide;
3-(3,4-dimethyl-1H-indol-1-yl)-N′-(3-methoxy-4-(pyridin-4-ylmethoxy)benzylidene)propanehydrazide;
3-(3,4-dimethyl-1H-indol-1-yl)-N′-(3-methoxy-4-(pyridazin-3-ylmethoxy)benzylidene)propanehydrazide;
N′-(3-methoxy-4-(pyridin-3-ylmethoxy)benzylidene)-2-phenylcyclopropanecarbohydrazide;
3-(3,4-dimethyl-1H-indol-1-yl)-N′-(3-methoxy-4-(pyrazin-2-ylmethoxy)benzylidene)propanehydrazide;
N′-((8-methoxyquinolin-6-yl)methylene)-2-phenylcyclopropane-carbohydrazide;
3-(1-methyl-1H-indol-3-yl)-N′-((8-methylquinolin-6-yl)methylene)-propanehydrazide;
2-phenyl-N′-((8-(trifluoromethyl)quinolin-6-yl)methylene)cyclopropanecarbohydrazide
N′-((8-chloroquinolin-6-yl)methylene)-2-phenylcyclopropane-carbohydrazide;
3-(6-methoxypyridin-2-yl)-N′-((8-methylquinolin-6-yl)methylene)propanehydrazide;
3-(3,4-dimethyl-1H-indol-1-yl)-N′-((8-methoxyquinolin-6-yl)methylene)propanehydrazide;
2-(3-chlorophenyl)-N′-((8-chloroquinolin-6-yl)methylene)-cyclopropanecarbohydrazide;
2-(3-chlorophenyl)-N′-((8-methylquinolin-6-yl)methylene)-cyclopropanecarbohydrazide;
2-(3-chlorophenyl)-N′-((8-methoxyquinolin-6-yl)methylene)-cyclopropanecarbohydrazide;
N′-((1,5-naphthyridin-2-yl)methylene)-2-(3-chlorophenyl)-cyclopropanecarbohydrazide;
3-(3-methyl-1H-indol-1-yl)-N′-(quinoxalin-6-ylmethylene)-propanehydrazide;
N′-((1,5-naphthyridin-2-yl)methylene)-3-(3-methyl-1H-indol-1-yl)propanehydrazide;
N′-(cinnolin-6-ylmethylene)-3-(3-methyl-1H-indol-1-yl)propanehydrazide;
2-(3-chlorophenylthio)-N′-((8-methylquinolin-6-yl)methylene)-acetohydrazide;
N′-(3,4-dimethoxybenzylidene)-2-(quinolin-2-yl)cyclopropane-carbohydrazide;
N′-(3,4-dimethoxybenzylidene)-2-(6-methylpyridin-2-yl)cyclopropanecarbohydrazide;
N′-(3,4-dimethoxybenzylidene)-2-(quinolin-3-yl)cyclopropane-carbohydrazide;
N′-(3,4-dimethoxybenzylidene)-3-(quinolin-3-yl)propanehydrazide;
2-(4-fluorophenyl)-N′-((8-methylquinolin-6-yl)methylene)-cyclopropanecarbohydrazide;
2-(6-methoxypyridin-3-yl)-N′-((8-methylquinolin-6-yl)methylene)-cyclopropanecarbohydrazide;
2-(6-methoxypyridin-2-yl)-N′-((8-methylquinolin-6-yl)methylene)cyclopropanecarbohydrazide;
2-(5-chloropyridin-2-yl)-N′-((8-methylquinolin-6-yl)methylene)-cyclopropanecarbohydrazide;
N′-((8-fluoroquinolin-6-yl)methylene)-2-phenylcyclopropane-carbohydrazide;
N′-((3,4-dihydro-2H-benzo[b][1,4]dioxepin-7-yl)methylene)-3-(3,4-dimethyl-1H-indol-1-yl)propanehydrazide;
N′-(3,4-dimethoxybenzylidene)-3-(4-methoxy-3-methyl-1H-indol-1-yl)propanehydrazide;
3-(4-chloro-3-((dimethylamino)methyl)-1H-indol-1-yl)-N′-(3,4-dimethoxybenzylidene)propanehydrazide;
N′-(3,4-dimethoxybenzylidene)-3-(1,7-dimethyl-1H-indol-3-yl)propanehydrazide;
3-(3-chloro-4-methyl-1H-indol-1-yl)-N′-(3,4-dimethoxybenzylidene)-propanehydrazide;
N′-(3,4-dimethoxybenzylidene)-3-(4-(hydroxymethyl)-3-methyl-1H-indol-1-yl)propanehydrazide;
2-(3-chlorophenylsulfonyl)-N′-(3,4-dimethoxybenzylidene)-propanehydrazide;
N′-((2-methoxy-4-methylquinolin-6-yl)methylene)-3-(3-methyl-1H-indol-1-yl)propanehydrazide;
2-(3-chlorophenylthio)-N′-((4,5-dimethoxypyridin-2-yl)methylene)propanehydrazide;
N′-(3,4-dimethoxybenzylidene)-3-(1H-indol-1-yl)propanehydrazide;
N′-(3,4-dimethoxybenzylidene)-2-(phenylthio)acetohydrazide;
N′-(3,4-dimethoxybenzylidene)-3-(3-methyl-1H-indol-1-yl)propanehydrazide;
N′-(3,4-dimethoxybenzylidene)-3-(4-methyl-1H-indol-1-yl)propanehydrazide;
N′-((3,4-dihydro-2H-benzo[b][1,4]di-oxepin-7-yl)methylene)-3-(3-methyl-1H-indol-1-yl)propanehydrazide;
3-(3-methyl-1H-indol-1-yl)-N′-(quinolin-6-ylmethylene)-propanehydrazide;
3-(3-methyl-1H-indol-1-yl)-N′-((4-methyl-3,4-dihydro-2H-benzo[b][1,4]-oxazin-7-yl)methylene)propanehydrazide;
3-(3-methyl-1H-indol-1-yl)-N′-((3-methylquinolin-6-yl)methylene)-propanehydrazide;
3-(3-methyl-1H-indol-1-yl)-N′-((4-methylquinolin-6-yl)methylene)-propanehydrazide;
N′-((4-methoxyquinolin-6-yl)methylene)-3-(3-methyl-1H-indol-1-yl)propanehydrazide;
3-(3,4-dimethyl-1H-indol-1-yl)-N′-((8-methylquinolin-6-yl)-methylene)propanehydrazide;
2-(3,4-dimethoxybenzylidene)-N-phenethylhydrazinecarboxamide;
2-(3-chlorophenylthio)-N′-(3,4-dimethoxybenzylidene)acetohydrazide;
N′-(3,4-dimethoxybenzylidene)-2-(3-(hydroxymethyl)phenylthio)-acetohydrazide;
3-(3-methyl-1H-indol-1-yl)-N′-(quinolin-7-ylmethylene)-propanehydrazide;
3-(3-methyl-1H-indol-1-yl)-N′-((2-methylquinolin-6-yl)methylene)-propanehydrazide;
or a pharmaceutically acceptable salt or a stereoisomer thereof.

76. The compound of claim 1, which is
trans-N′-(3,4-dimethoxybenzylidene)-2-(3-trifluoromethyl)phenyl)cyclopropane-carbohydrazide,
trans-N′-(3,4-dimethoxybenzylidene)-N-methyl-2-phenylcyclopropanecarbohydrazide,
trans-N′-((8-methylquinolin-6-yl)methylene)-2-phenylcyclopropane-carbohydrazide;
trans-N′-((8-methylquinolin-6-yl)methylene)-2-phenylcyclopropane-carbohydrazide, E1 enantiomer;
trans-N′-((8-methylquinolin-6-yl)methylene)-2-phenylcyclopropane-carbohydrazide, E2 enantiomer;
trans-N′-(3-methoxy-4-(pyridin-3-ylmethoxy)benzylidene)-2-phenylcyclopropanecarbohydrazide;
trans-N′-((8-methoxyquinolin-6-yl)methylene)-2-phenylcyclopropane-carbohydrazide;
trans-2-phenyl-N′-((8-(trifluoromethyl)quinolin-6-yl)methylene)-cyclopropanecarbohydrazide;
trans-N′-((8-chloroquinolin-6-yl)methylene)-2-phenylcyclopropane-carbohydrazide;
trans-2-(3-chlorophenyl)-N′-((8-chloroquinolin-6-yl)methylene)-cyclopropanecarbohydrazide;
trans-2-(3-chlorophenyl)-N′-((8-methylquinolin-6-yl)methylene)-cyclopropanecarbohydrazide;
trans-2-(3-chlorophenyl)-N′-((8-methoxyquinolin-6-yl)methylene)-cyclopropanecarbohydrazide;
trans-N′-((1,5-naphthyridin-2-yl)methylene)-2-(3-chlorophenyl)-cyclopropanecarbohydrazide;
trans-N′-(3,4-dimethoxybenzylidene)-2-(quinolin-2-yl)-cyclopropanecarbohydrazide;
trans-N′-(3,4-dimethoxybenzylidene)-2-(6-methylpyridin-2-yl)-cyclopropanecarbohydrazide;
trans-N′-(3,4-dimethoxybenzylidene)-2-(quinolin-3-yl)-cyclopropanecarbohydrazide;
trans-2-(4-fluorophenyl)-N′-((8-methylquinolin-6-yl)methylene)-cyclopropanecarbohydrazide;
trans-2-(6-methoxypyridin-3-yl)-N′-((8-methylquinolin-6-yl)methylene)-cyclopropanecarbohydrazide;
trans-2-(6-methoxypyridin-2-yl)-N′-((8-methylquinolin-6-yl)methylene)-cyclopropanecarbohydrazide;
trans-2-(5-chloropyridin-2-yl)-N′-((8-methylquinolin-6-yl)methylene)-cyclopropanecarbohydrazide;
trans-N′-((8-fluoroquinolin-6-yl)methylene)-2-phenylcyclopropane-carbohydrazide;
or a pharmaceutically acceptable salt thereof.

77. A pharmaceutical composition, comprising the compound of claim 1, or a pharmaceutically acceptable salt or stereoisomer thereof, and a pharmaceutically acceptable carrier.

78. A food product, comprising the compound of claim 1, or a pharmaceutically acceptable salt or stereoisomer thereof, and one or more food ingredients.

79. A cosmetic product, comprising the compound of claim 1, or a pharmaceutically acceptable salt or stereoisomer thereof, and one or more cosmetic ingredients.

80. A dental hygienic product, comprising the compound of claim 1, or a pharmaceutically acceptable salt or stereoisomer thereof, and one or more dental hygienic ingredients.

81. A method of inhibiting a taste, comprising administering to a subject in need of said taste inhibiting one or more compounds as defined in claim 1, or a pharmaceutically acceptable salt or a stereoisomer thereof.

82-92. (canceled)

93. A method of inhibiting the depolarization of a taste receptor cell, comprising contacting said taste receptor cell with one or more compounds of claim 1, or a pharmaceutically acceptable salt or stereoisomer thereof, wherein said compound is administered in an amount sufficient to inhibit depolarization of a taste receptor cell.

94. A method of preparing an improved pharmaceutical composition, wherein the improvement comprises adding to a pharmaceutical composition one or more compounds of claim 1, or a pharmaceutically acceptable salt or stereoisomer thereof.

95. A method of preparing an improved cosmetic product, wherein the improvement comprises adding to a cosmetic product a compound a compound of claim 1, or a pharmaceutically acceptable salt or stereoisomer thereof.

96. A method of enhancing insulin release from a cell, comprising contacting said cell with an effective amount a compound of claim 1, or a pharmaceutically acceptable salt or stereoisomer thereof.

97. The method of claim 96, wherein the cell is a non-human pancreatic cell.

98. The method of claim 96, wherein said cell is a human pancreatic cell.

99. The method of claim 96, wherein the compound is administered as a pharmaceutical composition or a veterinary composition.

100. The method of claim 96, wherein said cell is in vitro.

101. A method of enhancing insulin release in a mammal, comprising administering to the mammal in need of said enhanced insulin release an effective amount of a compound of claim 1, or a pharmaceutically acceptable salt or stereoisomer thereof.

102. A method of treating diabetes mellitus in a mammal, comprising administering to the mammal in need of said treatment an effective amount of a compound of claim 1, or a pharmaceutically acceptable salt or stereoisomer thereof.

103. A method of treating insulin resistance syndrome in a mammal, comprising administering to the mammal in need of said treatment an effective amount of a compound of claim 1, or a pharmaceutically acceptable salt or stereoisomer thereof.

104. A method of treating hyperglycemia in a mammal, comprising administering to the mammal in need of said treatment an effective amount of a compound of claim 1, or a pharmaceutically acceptable salt or stereoisomer thereof.

105. A method of enhancing GLP-1 release in a mammal, comprising administering to the mammal in need of said enhanced GLP-1 release an effective amount of a compound of claim 1, or a pharmaceutically acceptable salt or stereoisomer thereof.

106. A method of enhancing GLP-1 release from a cell, comprising contacting said cell with an effective amount of a compound of claim 1, or a pharmaceutically acceptable salt or stereoisomer thereof.

107. A method of decreasing gastric secretion and emptying in a mammal, comprising administering to the mammal in need thereof an effective amount of a compound of claim 1, or a pharmaceutically acceptable salt or stereoisomer thereof.

108. A method of inhibiting food intake in a mammal, comprising administering to a mammal in need thereof an effective amount of a compound of claim 1, or a pharmaceutically acceptable salt or stereoisomer thereof.

109. A method of decreasing glucagon secretion in a mammal, comprising administering to the mammal in need thereof an effective amount of a compound of claim 1, or a pharmaceutically acceptable salt or stereoisomer thereof.

110. A method of enhancing insulin sensitivity in a mammal, comprising administering to a subject in need thereof an effective amount of a compound of claim 1, or a pharmaceutically acceptable salt or stereoisomer thereof.

111. A method of treating obesity in a mammal, comprising administering to the mammal in need thereof an effective amount of a compound of claim 1, or a pharmaceutically acceptable salt or stereoisomer thereof.

112. A method of increasing beta cell mass of Langerhans in a mammal, comprising administering to the mammal in need thereof an effective amount of a compound of claim 1, or a pharmaceutically acceptable salt or a stereoisomer thereof.

113. A method of increasing insulin gene expression in a mammal, comprising administering to the mammal in need thereof an effective amount of a compound of claim 1, or a pharmaceutically acceptable salt or a stereoisomer thereof.

114. The method of claim 96, wherein said compound is N′-(3,4-dimethoxybenzylidene)-N-methyl-2-phenylcyclopropanecarbohydrazide, or a pharmaceutically acceptable salt of stereoisomer thereof.

115. The method of claim 114, wherein the compound is trans-N′-(3,4-dimethoxybenzylidene)-N-methyl-2-phenylcyclopropanecarbohydrazide or a pharmaceutically acceptable salt thereof.
